(19) United States
(12) Patent Application Publication
(10) Pub. No.: US 2021/0290132 A1
(43) Pub. Date: Sep. 23, 2021

(54) STRESS DISORDER TRAINING

(71) Applicant: The Medical Research, Infrastructure and Health Services Fund of the Tel Aviv Medical Center, Tel-Aviv (IL)

(72) Inventors: Talma HENDLER, Tel-Aviv (IL); Tom FRUCHTMAN-STEINBOK, Rehovot (IL); Avihai COHEN, Tel-Aviv (IL); Gal RAZ, Kfar Daniel (IL)

(73) Assignee: The Medical Research, Infrastructure and Health Services Fund of the Tel Aviv Medical Center, Tel-Aviv (IL)

(21) Appl. No.: 17/342,753

(22) Filed: Jun. 9, 2021

Related U.S. Application Data
(63) Continuation of application No. PCT/IL2019/051345, filed on Dec. 9, 2019.
Application No. PCT/IL2019/051345 is a continuation-in-part of application No. 17/319,265, filed on May 13, 2021.
Application No. 17/319,265 is a continuation of application No. PCT/IL2019/051245, filed on Nov. 14, 2019.
(60) Provisional application No. 62/777,187, filed on Dec. 9, 2018.
(60) Provisional application No. 62/767,650, filed on Nov. 15, 2018.

(51) Int. Cl. A61B 5/16; A61B 5/00; A61B 5/377; A61B 5/375; A61B 5/372; G16H 20/70

(52) U.S. Cl. CPC A61B 5/165; A61B 5/4884; A61B 5/377; G01R 33/4806; A61B 5/372; G16H 20/70; A61B 2505/09; A61B 5/375

(57) ABSTRACT

A method for training a subject diagnosed with a stress disorder caused by a trauma, including: selecting a challenge expected to trigger a symptom of the stress disorder in the subject; exposing the subject to the challenge; recording electrical signals generated by the brain of the subject by at least one electrode, in conjunction with the exposing; processing the recorded electrical signals to estimate an activation level of at least one specific brain region; presenting at least one indication of the estimated activation level to the subject; repeating the recording, the processing and the presenting.

### RELATED APPLICATIONS

This application is a Continuation of PCT Patent Application No. PCT/IL2019/051345 having International filing date of Dec. 9, 2019, which claims the benefit of priority under 35 USC § 119(e) of U.S. Provisional Patent Application No. 62/777,187 filed on Dec. 9, 2018.

This application is also a Continuation-in-Part (CIP) of U.S. patent application Ser. No. 17/319,265 filed on May 13, 2021, which is a Continuation of PCT Patent Application No. PCT/IL2019/051245 having International filing date of Nov. 14, 2019, which claims the benefit of priority under 35 USC § 119(e) of U.S. Provisional Patent Application No. 62/767,650 filed on Nov. 15, 2018.

The contents of the above applications are all incorporated by reference as if fully set forth herein in their entirety.

### FIELD AND BACKGROUND OF THE INVENTION

The present invention, in some embodiments thereof, relates to stress disorders training and, more particularly, but not exclusively, to post traumatic stress disorder training.

### SUMMARY OF THE INVENTION

Some examples of some embodiments of the invention are listed below. It should be noted that features from any example may be used in combination with features described in other examples:
Example 1. A method for training a subject diagnosed with a stress disorder, comprising: exposing a subject diagnosed with a stress disorder to a trauma-related challenge; delivering a feedback to said subject during said exposing regarding an activity level of one or more brain regions and/or neural circuits.
Example 2. A method according to example 1, wherein said exposing to said trauma-related challenge affects activation of said one or more brain regions and/or neural circuits.
Example 3. A method according to example 1, comprising:
recording electrical signals from said subject during or following said exposing;
generating an electrical finger print (EFP) based on at least a portion of said recorded electrical signals, which relates to a current activation level of said one or more brain regions and/or neural circuits;
wherein said delivering comprises delivering a feedback according to said current activation level of said one or more brain regions and/or neural circuits.
Example 4. A method according to example 3, wherein said delivering a feedback comprises modifying said trauma-related challenge according to said current activation level.
Example 5. A method according to any one of examples 3 or 4, wherein said electrical signals are EEG and/or EMG electrical signals.
Example 6. A method according to any one of the previous examples, wherein said trauma-related challenge comprises a challenge selected to activate an amygdala and/or an amygdala related neural circuit.
Example 7. A method according to any one of the previous examples, wherein said trauma-related challenge comprises a challenge personalized to a specific trauma of said subject and/or to a specific stress disorder.
Example 8. A method according to any one of the previous examples, wherein said stress disorder comprises post-traumatic stress disorder (PTSD).
Example 9. A method according to any one of the previous examples, wherein said trauma-related challenge is selected to upregulate activation of said one or more brain regions, and wherein said feedback is delivered according to an ability of said subject to downregulate said activation level.
Example 10. A method according to any one of the previous examples, wherein said delivering comprises delivering said feedback to said subject according to an ability of said subject to cope with said trauma-related challenge.
Example 11. A method for training a subject diagnosed with a stress disorder, comprising:
exposing a subject diagnosed with a stress disorder to a non-specific stress challenge;
evaluating said subject ability to cope with said non-specific stress challenge;
providing a stress-related challenge personalized to said subject according to said evaluating results.
Example 12. A method according to example 11, wherein said exposing comprises exposing said subject to a non-specific stress challenge selected to activate one or more brain regions, and wherein said evaluating comprises evaluating said subject to cope with activation of said one or more brain regions by said non-specific stress challenge.
Example 13. A method according to example 12, wherein said evaluating comprises evaluating said subject ability to affect the activation of said one or more brain regions.
Example 14. A method according to any one of example 12 or 13, wherein said non-specific stress challenge is configured to upregulate an activity of an amygdala and/or an amygdala-related neural circuit.
Example 15. A method according to example 14, wherein said evaluating comprises evaluating an ability of said subject to downregulate the activity of said amygdala and/or said amygdala-related circuit during said exposing.
Example 16. A method for training a subject diagnosed with a stress disorder, comprising: exposing a subject diagnosed with a stress disorder to a stress-related challenge;
providing a feedback to said subject by modifying said stress-related challenge during said exposing.
Example 17. A method according to example 16, comprising:
determining an activity level of one or more brain regions following said exposing, and wherein said providing comprises providing said feedback by modifying said stress—related challenge according to said determined activity level.
Example 18. A method according to example 17, wherein said determining comprises determining that an activity level of said one or more brain regions is downregulated, and wherein said providing comprises reducing an intensity of said stress-related channel.
Example 19. An apparatus program configured to provide one or more of exposing, evaluating and/or delivering feedback according to any one of the previous claims.

Some additional examples of some embodiments of the invention are listed below. It should be noted that features from any example may be used in combination with features described in other examples:

Example 1. A method for training a subject diagnosed with a stress disorder caused by a trauma, comprising:
selecting a challenge expected to trigger a symptom of said stress disorder in said subject; exposing said subject to said challenge;
recording electrical signals generated by the brain of said subject by at least one electrode, in conjunction with said exposing;
processing said recorded electrical signals to estimate an activation level of at least one specific brain region;
presenting at least one indication of said estimated activation level to said subject;
repeating said recording, said processing and said presenting.
Example 2. A method according to example 1, wherein said exposing to said challenge affects an activation level of said at least one specific brain region.
Example 3. A method according to any one of examples 1 or 2, comprising:
identifying a relation between at least a portion of said recorded electrical signals and an electrical fingerprint indicating an activation level of said at least one brain region, and wherein said generating comprises generating said at least one indication based on said identified relation.
Example 4. A method according to any one of the previous examples, wherein said presenting comprises modifying said challenge according to said activation level of said at least one brain region.
Example 5. A method according to any one of the previous examples, wherein said at least one brain region is a brain region of the limbic system that has a volume of less than 20% from the volume of the limbic system.
Example 6. A method according to any one of the previous examples, wherein said challenge comprises a challenge selected to activate an amygdala and/or brain regions connected to the amygdala by a neural network.
Example 7. A method according to any one of the previous examples, wherein said at least one specific brain region comprises two or more specific brain regions.
Example 8. A method according to any one of the previous examples, wherein said at least one specific brain region does not comprise the Amygdala.
Example 9. A method according to any one of the previous examples, wherein said stress disorder comprises post-traumatic stress disorder (PTSD).
Example 10. A method according to any one of the previous examples, wherein said challenge is selected to upregulate activation of said at least one specific brain region, and wherein said at least one indication is presented according to an ability of said subject to downregulate said activation level.
Example 11. A method according to any one of the previous examples, comprising:
instructing said subject to perform at least one exercise selected to affect an activation level of said at least one specific brain region.
Example 12. A method according to example 11, wherein said presenting comprises presenting said at least one indication according to an ability of said subject to modulate an activation level of said at least one specific brain region by performing said at least one exercise.
Example 13. A method according to any one of the previous examples, wherein said in conjunction with said exposing comprises before, during and after said exposing.
Example 14. A method for selecting a subject for a stress disorder training, comprising: evaluating a subject diagnosed with a stress disorder caused by a trauma, to identify at least one trauma-related challenge expected to trigger said trauma in said subject and at least one non-specific stress challenge not expected to trigger said trauma in said subject;
exposing said subject to said at least one non-specific stress challenge;
evaluating during and/or following said exposing an ability of said subject to self-modulate an activation level of at least one specific brain region affected by said trauma;
providing said at least one trauma-related challenge to said subject according to said evaluating results.
Example 15. A method according to example 14, wherein said stress disorder comprises post-traumatic stress disorder (PTSD).
Example 16. A method according to any one of examples 14 or 15, wherein said at least one non-specific stress challenge is configured to upregulate an activity of an amygdala and/or an amygdala-related neural circuit.
Example 17. A method according to example 16, wherein said evaluating comprises evaluating an ability of said subject to downregulate the activity of said amygdala and/or said amygdala-related circuit during and/or following said exposing.
Example 18. A method according to any one of examples 14 to 17, comprises:
recording electrical signals from said subject by at least one electrode during and/or following said exposing, wherein at least a portion of said recorded electrical signals indicate an activity level of said at least one specific brain region, and wherein said evaluating comprises evaluating said subject ability to affect the activation of said at least one specific brain region based on said recorded electrical signals.
Example 19. A method according to example 18, wherein said electrical signals comprises EEG signals recorded by at least one EEG electrode.
Example 20. A method according to any one of examples 14 to 19, wherein said at least one specific brain region does not comprise the Amygdala.
Example 21. A method for training a subject diagnosed with a stress disorder caused by a trauma, comprising:
selecting a challenge expected to trigger said trauma in said subject;
exposing said subject to said challenge;
measuring said subject response to said challenge;
providing a feedback to said subject by modifying said challenge according to said measured response.
Example 22. A method according to example 21, wherein said measuring comprises determining an activation level of at least one specific brain region, and wherein said providing comprises providing said feedback to said subject by modifying said challenge according to said determined activation level.
Example 23. A method according to any one of examples 21 or 22, comprising:
recording electrical signals from said subject by at least one electrode in conjunction with said exposing, wherein at least a portion of said recorded electrical signals indicate an activity level of said at least one specific brain region, and wherein said determining comprises determining said activation level of said at least one specific brain region based on said recorded electrical signals.
Example 24. A method according to example 23, wherein said electrical signals comprises EEG signals recorded by at least one EEG electrode.
Example 25. A method according to example 24, wherein said determining comprises identifying a relation between at least a portion of said recorded EEG signals and a least one EEG signature or indication thereof stored in a memory, wherein said at least one EEG signature indicates an activity level of at said at least one specific brain region.
Example 26. A method according to any one of examples 23 to 25, wherein said in conjunction with said exposing comprises before, during and/or after said exposing.
Example 27. A method according to any one of examples 23 to 26, wherein said at least one specific brain region comprises the Amygdala.
Example 28. A method according to any one of examples 21 to 27, wherein said exposing comprises exposing said subject diagnosed with a stress disorder to a stress-related challenge while said subject is not confined by an imaging system.
Example 29. A method for training a subject diagnosed with a stress disorder caused by a trauma, comprising:
assessing at least one impaired neurobehavioral process of said stress disorder in said subject;
selecting a challenge according to said identified impaired neurobehavioral process;
exposing said subject to said selected challenge;
recording electrical signals generated by the brain of said subject by at least one electrode, in conjunction with said exposing;
processing said recorded electrical signals to estimate an activation level of at least one specific brain region;
presenting at least one indication of said estimated activation level to said subject;
repeating said recording, said processing and said presenting.
Example 30. A method according to example 29, comprising specifically designing said challenge according to said impaired neurobehavioral process.
Example 31. A method according to any one of examples 29 or 30, comprising evaluating an expression of at least one symptom of said stress disorder in said subject, and wherein said assessing comprises assessing said at least one impaired neurobehavioral process based on said expression of said at least one symptom.
Example 32. A method according to any one of examples 29 to 31, wherein said at least one impaired neurobehavioral process comprises at least one of an impaired stress detection process, an impaired emotion regulation process, and an impaired fear extinction process.
Example 33. A method according to any one of examples 29 to 32, wherein said stress disorder comprises post-traumatic stress disorder (PTSD).
Example 34. A method according to any one of examples 29 to 33 comprising, instructing said subject to perform at least one exercise in a timed relation with said exposing, wherein said at least one exercise is configured to affect an activation level of said at least one specific brain region.
Example 35. A method according to any one of examples 29 to 34, comprises determining an activation level of said at least one specific brain region by identifying a relation between at least a portion of said recorded electrical signals and at least one electrical signature or indication thereof stored in a memory, wherein said at least one electrical signature or said indication thereof, indicate an activation level of said at least one brain region.
Example 36. A method according to any one of examples 29 to 35, wherein said at least one specific brain region comprises a brain region of the limbic system having a volume which is less than 20% of the volume of the limbic system.
Example 37. A method according to any one of examples 29 to 36, wherein said at least one specific brain region comprises the Amygdala.
Example 38. A method according to any one of examples 29 to 37, wherein said exposing comprises exposing said subject diagnosed with a stress disorder to said impaired neurobehavioral process-related challenge while said subject is not confined by an imaging system.
Example 39. A method for treating depression or anxiety, comprises:
providing a subject diagnosed with PTSD and depression or PTSD and anxiety;
training said subject using neurofeedback to control activation of at least one specific brain region to reduce severity of PTSD, thereby improving depression or anxiety symptoms.
Example 40. A method according to example 36, comprising:
evaluating said depression or said anxiety in said subject following said training; modifying said neurofeedback training according to said evaluation results.
Example 41. A method according to any one of claim 39 or 40, comprising:
adjusting an existing drug regime of at least one bioactive compound for treating depression or anxiety in said subject or setting a new drug regime including at least one bioactive compound for treating said depression or said anxiety in said subject, based on a success of said training.
Example 42. A method according to any one of examples 39 to 41, wherein neurofeedback training comprises:
exposing said subject to a challenge related to a trauma of said subject;
instructing said subject to perform at least one exercise in conjunction with said exposing, wherein said at least one exercise is configured to affect an activation level of at least one specific brain region;
recording electrical signals from said subject during or following said exposing by at least one electrode, wherein at least a portion of said recorded electrical signals indicate an activation level of said at least one specific brain region;
delivering a feedback to said subject in conjunction with said exposing regarding an activation level of said at least one specific brain region based on said recorded electrical signals.
Example 43. A method according to example 42, wherein said electrical signals comprise EEG electrical signals.
Example 44. A method according to any one of examples 39 to 43, wherein said training is performed while said subject is not confined by an imaging system.
Example 45. A method for preparing a subject for an expected stress disorder trigger, comprising:
providing a list of exercises which modulate an activation of at least one specific brain region;
receiving an indication prior to exposure of a subject to a stress-disorder trigger;
instructing a subject diagnosed with said stress disorder to perform at least one exercise from said list of exercises.
Example 46. A method according to example 42, comprising:
recording EEG signals generated by the brain of said subject;
estimating an activation level of said at least one specific brain region based on said recorded EEG signals;
presenting at least one indication of said estimated activation level to said subject.
Example 47. A device for delivery of a stress disorder training, comprising:
a memory;
a control circuitry functionally coupled to said memory, configured to:
deliver to said subject a challenge configured to trigger at least one symptom of said stress disorder in said subject;
receive at least one electrical signal generated by the brain of said subject;
evaluate using said received electrical signals an effect of said challenge on at least one specific brain region, and an effect of said subject on an activation level of said at least one specific brain region;
automatically generate at least one human detectable indication based on the results of said evaluation.
Example 48. A device according to example 47, comprising a user interface, wherein a user selects said challenge using said user interface from a list of challenges stored in said memory.
Example 49. A device according to example 47, wherein said control circuitry is configured to select said challenge from a list of challenges stored in said memory.
Example 50. A device according to any one of examples 47 to 49, wherein said control circuitry is configured to process said effect to determine how to modulate said challenge.
Example 51. A device according to any one of claims examples 47 to 50, wherein said at least one electrical signal comprises an EEG signal.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determine activation level of a brain region, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a process for a stress disorder neurofeedback (NF) training, for example an electrical fingerprint neurofeedback (EFP-NF) training process from a patient perspective, according to some embodiments of the invention;

FIG. 2A is a flow chart of a NF training process, for example an EFP-NF training process, according to some embodiments of the invention;

FIG. 2B is a schematic illustration showing types of assessment of a subject, for example a subject diagnosed with a stress disorder, according to some exemplary embodiments of the invention;

FIG. 2C is a schematic representation of a matrix showing a relation between some symptoms of a stress-disorder, for example PTSD, and impaired neurobehavioral processes, according to some exemplary embodiments of the invention;

FIGS. 2D, 2E and 2F are schematic representations of interfaces delivered to a trainee generated based on a trainee's symptoms and/or impaired neurobehavioral processes, according to some exemplary embodiments of the invention;

FIG. 2G is a flow chart of a NF training process personalized according to a trainee's stress disorder related symptoms and/or impaired neurobehavioral processes, according to some exemplary embodiments of the invention;

FIGS. 2H, 2J, 2I and 2K are graphs showing changes in stress disorder-related symptoms measured using CAPS-5 subscales following NF training, according to some exemplary embodiments of the invention;

FIG. 3 is a flow chart of a process for providing a feedback to a patient during a NF training process by modifying an exposure interface, for example a trauma related exposure interface of the NF training, according to some embodiments of the invention;

FIG. 4 is a block diagram of a system for delivery of NF training, for example EFP-NF training, according to some embodiments of the invention;

FIG. 5 is an outline of a validation experiment which details the overall procedure of the trial from recruitment to follow up;

FIG. 6 is a table showing demographic and baseline clinical information about the group of participants in the experiment;

FIG. 7 describes an overall order of intervention sessions by intervention group;

FIG. 8 describes a protocol for an animated scenario neutral interface;

FIG. 9 describes an auditory neutral interface;

FIG. 10 describes a protocol section detailing a gradual exposure of patients in the exposure group to the content of their individual trauma; Patients begin with training in a neutral context and upon reaching a skill criterion they move on to train using their individual trauma in the exposure sessions;

FIG. 11 is a graph showing an example of changes in EFP signal during the first exposure session of one patient in the exposure group;

FIGS. 12A, 12B, 12C, 12D, 12E, 12F and 12G are graphs showing changes in EFP signals between different sessions during the experiment of the patient appearing in FIG. 11;

FIG. 13 is a graph showing changes in EFP Z score average during 13 weeks of NF training in the experiment;

FIGS. 14A-14B are graphs showing changes in EFP Z score average between the different groups in the experiment;

FIG. 15 is a graph showing changes in CAPS-5 total score in three groups of participants in the experiments following the NF training;

FIG. 16A is a graph showing changes in CAPS-5 total score in individual trainees received EFP-NF in a neutral context during the experiment;

FIG. 16B is a graph showing changes in CAPS-5 total score in individual trainees received EFP-NF in an exposure context during the experiment;

FIG. 16C is a graph showing percentage of participants in each group of the experiment showing an improvement in CAPS-5 total score which is larger than 5 points;

FIG. 17 describes changes in different CAPS 5 subscales between different groups of the experiment;

FIG. 18 is a graph describing changes in PCL score between different groups of the experiment;

FIG. 19 shows target, for example amygdala engagement following the training;

FIG. 20 is a graph showing correlation between EFP training success index and subsequent BOLD activation during real-time fMRI neurofeedback. This positive correlation demonstrates that participants who were very successful at down regulating their EFP signal during training sessions (i.e. overall best performance session) were also better at down regulating their amygdala BOLD signal during real-time neurofeedback following intervention;

FIG. 21A is a schematic representation of a process of an experiment;

FIG. 21B is a schematic representation of training sessions performed in the experiment of FIG. 21A;

FIG. 22 shows two graphs describing a learning effect of the NF training demonstrated as changes in the Amygdala EFP signal during the NF training performed in the experiment of FIG. 21A, according to some exemplary embodiments of the invention;

FIG. 23A is a graph showing changes in Total CAPS-5 scores following the NF training performed in the experiment of FIG. 21A;

FIG. 23B is a graph showing changes Total CAPS-5 Score Percent Symptom Reduction from TP1 to TP2 following NF training performed in the experiment of FIG. 21A;

FIG. 23C is a graph showing changes in total PCL assessed following NF training performed in the experiment of FIG. 21A;

FIG. 23D is a graph showing changes in total PCL through the experiment and in follow up meeting as assessed following NF training performed in the experiment of FIG. 21A;

FIG. 24A is a graph and a schematic representation of a brain showing rtfMRI-NF Target Engagement in the experiment of FIG. 21A;

FIG. 24B is a CONSORT flow chart of the experiment of FIG. 21A;

FIG. 25 is a schematic representation of an rtfMRI-NF paradigm;

FIG. 26A is a graph showing changes in BDI-II score following NF training; and

FIG. 26B is a graph showing changes in STAI score following NF training.

### DESCRIPTION OF SPECIFIC EMBODIMENTS OF THE INVENTION

The present invention, in some embodiments thereof, relates to stress disorders training and, more particularly, but not exclusively, to post traumatic stress disorder training.

Post-traumatic stress disorder (PTSD) is characterized, for example, by excessive emotion reactivity and diminished emotion regulation, corresponding to changes in activation levels of at least one brain region, for example, hyperactive amygdala and hypoactive ventro-medial prefrontal cortex (vmPFC). One of the problems in treating PTSD, is non-specific targeting of these process abnormalities. In some embodiments of the invention, a neurofeedback (NF) intervention, for example an electrical finger print (EFP)-NF for PTSD patients is applied, aimed at down-regulating amygdala activity or other stress-associated regions. In some embodiments, in order to target a disorder-specific process an individually-tailored trauma-related content as the learning feedback is applied, allowing for personalized process-based NF. In some embodiments, an electrical finger print (EFP) is generated for example as described in U.S. patent application Ser. No. 13/983,419.

According to some embodiments, a brain region is a region of the brain which is part of the different neuroanatomical hierarchies of the brain, for example a first hierarchy, a second hierarchy and a third hierarchy of neuroanatomical regions in the brain. In some embodiments, the brain region is a region of the brain located in at least one of the hindbrain, the midbrain, and the forebrain, for example in a second hierarchy or a third hierarchy. In some embodiments, a brain region is at least one brain region of the limbic system, for example a brain region of the limbic system having a volume which is less than 20%, less than 10%, less than 5% or any intermediate, smaller or larger percentage value of the total volume of the limbic system. In some embodiments, a brain region has a volume which is less than 10%, less than 8%, less than 5%, less than 1% or any intermediate, smaller or larger percentage value of the total volume of the brain. In some embodiments, the brain region is part of at least one of the limbic system, the salience network, the mesolimbic system, the reward circuit, the prefrontal control circuit, the ventromedial prefrontal cortex, the brainstem, the threat circuit and the cerebellum.

According to some embodiments, PTSD patients are assessed before and/or after the EFP-NF treatment for example, to determine clinical severity using CAPS-5 and/or PCL assessment methods. Optionally, the PTSD patients are assessed for neural-target engagement using amygdala fMRI-NF.

According to some embodiments, stress-disorder diagnosed patients treated with the EFP-NF training, for example using an EFP-NF with a personalized trauma interface, down-regulate the AmygEFP signal, and/or activity of at least one stress-related brain region by performing exercises learned during the EFP-NF, optionally without receiving feedback.

An aspect of some embodiments of the invention relates to training a subject suffering from stress disorders, for example post-traumatic stress disorders (PTSD) using neurofeedback (NF) training, for example electrical finger print-neurofeedback (EFP-NF). In some embodiments, the electrical finger print (EFP) is an electroencephalography (EEG) fingerprint related to an activity level of one or more brain regions, for example as described in US20140148657A1. In some embodiments, the EFP-NF training affects activation levels of brain regions and/or neural circuits related to the stress disorders, for example to a memory of the stress-disorders trigger. In some embodiments, the EFP-NF affects activation levels of brain regions and/or neural circuits related to a memory of a traumatic event, for example a traumatic event that was a trigger of the stress disorder, for example a PTSD. In some embodiments, the stress disorders comprise Agoraphobia Without History of Panic Disorder, Social Phobia, Specific Phobia, Obsessive-Compulsive Disorder, Depression, Substance Abuse.

According to some embodiments, the electrical fingerprint (EFP) comprises an EEG electrical fingerprint of the amygdala and/or other brain regions related to one or more stress disorders, for example PTSD. In some embodiments, the EFP-NF training is used to selectively affect the stress disorders-related brain regions and/or neural circuits. In some embodiments, the EFP-NF training reduces activation levels of the stress disorders-related brain regions and/or neural circuits. Alternatively or additionally, the EFP-NF training increases activation levels of the stress disorders-related brain regions and/or neural circuits. In some embodiments, the stress disorders, for example PTSD, EFP-NF training is used to reduce activation of the amygdala.

According to some embodiments, during the EFP-NF training, EEG signals are recorded from a subject, for example a trainee. In some embodiments, a trainee is a patient diagnosed with a stress disorder. In some embodiments, at least a portion of the recorded EEG signals is analysed, for example to determine a current activity state of a selected brain region, for example the amygdala. In some embodiments, a stored EEG electrical fingerprint associated with a specific activity level of the brain region is used to determine a current activity level of the brain level, for example by associating a recorded EEG signal with a stored EEG signal.

According to some embodiments, during the EFP-NF training an activity level of a selected brain region, for example the amygdala is monitored. In some embodiments, the trainee learns how to modulate the activity of the brain regions, for example how to upregulate the activity and/or how to downregulate the activity of the brain region. In some embodiments, the trainee receives a feedback on his success to reach a desired activity level.

According to some embodiments, the feedback is online, for example while the subject performs one or more exercises to modulate the activity of the brain region. Alternatively or additionally, the feedback is continuous. In some embodiments, the feedback is provided to the subject and/or to a supervisor which monitors the EFP-NF process. In some embodiments, the feedback is provided by modifying an interface, for example a scenario, presented to the subject. In some embodiments, the feedback is provided by modifying an interface that is designed to upregulate and/or downregulate the activation of the brain region. In some embodiments, the feedback is delivered to the subject in less than 10 seconds, for example less than 5 seconds, less than 2 seconds, or any intermediate, smaller or larger value from recording the EEG signals. Additionally, the feedback is a continuous feedback, for example the feedback is delivered during a time period of at least 30 seconds and related to changes in the activation levels of the brain region during this time period,

According to some embodiments, the stress disorders EFP-NF training is combined with exposure therapy. In some embodiments, in the combined NF training, an interface, for example a challenge is provided to the subject during the EFP-NF training is related to a specific stress disorder, for example to a specific trigger of the stress disorder. In some embodiments, a subject is gradually exposed to a trigger of the stress disorder during the combined EFP-NF training. In some embodiments, an interface is gradually modulated to include an increasing number of features related to the trauma. Alternatively or additionally, an exposure time of a subject to the trauma-related features increases. In some embodiments, the trauma-related features are selected to evoke a response of a trainee to a specific trauma, for example activation of one or more brain regions and/or neural circuits.

According to some embodiments, the challenge provided to the subject is specific to a stress disorder, for example: for PTSD the challenge comprises sounds, sensations and/or scenes that cause triggering, delivered for example by virtual reality (VR) or augmented reality (AR); for Agoraphobia Without History of Panic Disorder the challenge comprises visualization of open space, delivered for example by VR or AR; for Social Phobia, the challenge comprises social interaction, images of faces asking questions, getting vague critic; for Specific Phobia the challenge comprises spiders; for depression which involves suicide obsession, the challenge comprises features related to suicide; for Substance Abuse, the challenge comprises image of drug paraphernalia.

According to some embodiments, the NF training, for example the EFP-NF training is used to train a subject suffering from stress disorders, for example PTSD, to down regulate activation of one or more brain regions and/or neural circuits that are selectively activated. In some embodiments, the one or more brain regions and/or neural circuits, for example the amygdala, are selectively activated during the NF training. In some embodiments, an interface delivered to the subject, for example a subject suffering from a stress-disorder comprises one or more features related to a trigger of the stress disorder. In some embodiments, the one or more features comprise sound, visual, smell and/or a sensation related to the stress disorder.

A potential advantage of receiving online and continuous feedback on the ability of a subject to control the activation of stress-related brain regions, for example during an exposure to a personalized trauma trigger, is that it allows an efficient and continuous training process with much higher temporal resolution, compared to, for example NF training methods which are based on MRI, for example fMRI. An additional potential advantage of receiving online and continuous feedback on a subject ability to control activation of brain regions, while exposing the subject to a personalized trauma trigger, is that it allows to stop the exposure of the subject to the trauma trigger in less than 10 seconds, for example in less than 5 seconds, in less than 2 seconds or any intermediate, smaller or larger value, from recording EEG signals related to an undesired activity level.

An aspect of some embodiments relates to examining an ability of a subject to control an activation level of one or more brain regions and/or neural circuits before exposing the subject to a stress-related trigger, for example a trauma-related trigger, specific to the subject. In some embodiments, a subject suffering from a stress disorder, for example PTSD, is exposed to a non-specific stress trigger prior to an exposure to a stress-trigger specific to the subject.

According to some embodiments, the specific, for example personalized, stress-trigger selectively activates, for example selectively upregulates, the activation of one or more brain regions and/or neural circuits related to emotion control and/or trauma memory. In some embodiments, an ability of the subject to control the activation, for example to downregulate the activation of the one or more brain regions and/or neural circuits expected to be activated by the specific stress trigger, is examined prior to the exposure to the personalized stress trigger, for example a personalized trauma trigger.

According to some embodiments, a supervisor, for example a psychologist, a psychiatrist, or a mental health expert, monitors reactions of the subject, for example physiological and/or behavioral reactions of the subject to the non-specific stress trigger and/or to the stress specific trigger, for example the trauma-specific trigger. In some embodiments, the supervisor monitors online the reactions of the subject during the NF training, for example during the EFP-NF training. In some embodiments, the supervisor modifies one or more parameters of the NF training according to the reactions of the subject. Alternatively, a control circuitry of a system automatically modifies the one or more parameters of the NF training. In some embodiments, the one or more parameters comprise an interface, for example a stress trigger interface, of the NF training which delivers the specific and non-specific stress trigger to the subject, for example an audio and/or visual scenario, sound, smell, and/or sensation included in the interface. In some embodiments, the interface comprises a virtual reality (VR) or an augmented reality (AR) interface. In some embodiments, the one or more parameters of the NF training comprises overall training duration, duration of each training session, intermission period between training sessions, content and/or type of the stress trigger interface, moving from a non-specific stress trigger interface to a specific stress trigger interface, for example an interface that is personalized to trigger a specific trauma event or a trauma memory specific to the subject.

According to some embodiments, a subject is continuously exposed to a traumatic event, for example to a trigger of the traumatic event, during an EFP-NF training procedure. In some embodiments, during the EFP-NF training procedure, the provided exposure is modulated according to the success of the subject to reach a desired activity level while being exposed to the traumatic event. In some embodiments, a continuous exposure and exposure modulation is generated by a closed-loop feedback during the NF raining session.

According to some embodiments, the exposure comprises traumatic contents extracted for each individual through a prior detailed interview, for example an interview which is part of the diagnosis of the stress disorder, for example PTSD. In some embodiments, a trauma narrative is presented via auditory, visual and/or other sensory modalities. In some embodiments, the sensory modalities are relevant to the emotionality/stress of the trauma, for example smell, touch, visual or auditory cue. In some embodiments, the presentation comprises not immersive (natural) environments. Alternatively, the presentation comprises immersive environments, for example virtual or augmented reality environments.

According to some embodiments, the traumatic content is introduced gradually in terms of an emotional intensity of the content, for example the gradual introduction of the traumatic content is personalized per trainee, optionally prior to the NF training. In some embodiments, the traumatic content is introduced gradually by using an auditory interface, for example a voice of another person describing a traumatic event in a second person manner, then specific sounds are added that could be part of the traumatic memory, for example crying, yelling etc. Alternatively or additionally, the traumatic content is introduced gradually by using a visual interface, for example showing graphic presentations of the narrative, then introduce personal cues related to objects, colors or people involved in the story.

According to some embodiments, feedback is provided by continuous modulation of the exposure to the traumatic event and/or modulation of the gradual introduction of the traumatic content. In some embodiments, the feedback is provided by constantly monitoring EFP signals based on recorded EEG signals during a EFP-NG training session. In some embodiments, the feedback, for example a sensory rewarding feedback, is provided according to a change of the signals in a desired direction, for example down or up regulation relative to a baseline or previous signals values.

According to some embodiments, reward is perceived through decreased exposure intensity, for example by reducing clarity of sensory presentations or intensity of content, and/or changing the narrative to become more distant to a trauma focus. In some embodiments, the feedback comprises a continuous feedback. Alternatively, the feedback comprises an intermittent feedback presented to a trainee every 2-30 seconds, for example every 2-15 seconds, every 10-20 seconds, every 17-30 seconds or any intermediate, smaller or larger range of values. In some embodiments, the trainee perceives the traumatic content for a selected time period in a range of 5-90 seconds, for example 5-30 seconds, 20-60 seconds, 50-90 seconds or any intermediate, smaller or larger range of values. In some embodiments, the trainee perceives the traumatic content while trying to modulate brain signals and gets the feedback about success on a separate screen, for example as described above. Alternatively or additionally, the feedback is delivered by presenting a scale, for example a metric scale showing degree of success in change.

Without being bound by any theory, the idea of patient specific context during NF training may serve two principles: one is related to better identification of the relevant brain circuit. Even though one or more brain regions are targeted, by providing the specific content while modulating these one or more brain regions, the most relevant pathological mechanism may be recruited. The second is related to the feedback mechanism during NF. A positive feedback usually involves a reward circuitry, but when the reward is a positive/desirable change in the traumatic context, it leads to new learning that attach traumatic content with a reward, thus contradict its threat automatic meaning.

According to some embodiments, the NF training, for example the EFP-NF training is a gradual procedure, for example a trainee first establishes its skill to modulate a target brain signal in a neutral (not disease specific) context, and only then confronted with the disease (and distressing) context, for example a trauma narrative or other. In some embodiments, in the first phase of the intervention, the trainee receives one or more NF sessions with a non-specific feedback, for example an audio or an audio-visual animated scenario. In some embodiments, trainees who succeed in lowering their EFP signal during these one or more sessions continue to a second phase of the NF training. In some embodiments, the second phase is performed during exposure to diseases specific contents and contexts, for example trauma specific content and context.

According to some embodiments, a criterion for when moving to the distressing content is set in order to ensure that during exposure sessions trainees will be able to use their already established techniques to probe the relevant brain signal and modulate it despite the challenging interference.

A potential advantage of using EFP-NF is that it allows to use multiple phases, multiple sessions, online and optionally continuous feedback and close monitoring by a supervisor, whereas in other brain region activation NF techniques, for example fMRI-NF, these abilities are largely limited. The EFP provides both the spatial information from the fMRI and the accessibility of the EEG.

According to some embodiments, when first exposed to the distressing content, a supervisor, for example a clinician attends the training room, closely monitor and reassures that the trainee is not overwhelmed with the situation and is able to handle the training. In some embodiments, a trained clinician is present also to guide the patient when confronting the distressing environment. In some embodiments, after each cycle of NF training, the clinician allows the patient to share their experience and discuss the mental strategy that was used successfully. Additionally or alternatively, at the end of each session, a learning graph is presented to the trainee and optionally, the clinician discusses with the trainee the success points in details. These safety steps are easily implemented in an EEG-NF set up, but are practically impossible with fMRI-NF.

According to some embodiments, an exposure narrative which is played back to the trainee as the feedback is a personalized exposure narrative generated for each individual subject. In some embodiments, prior to initiating a NF training, a trained clinician interviews and records the patient about his/her particular traumatic event. In some embodiments, following the interview the information is edited, for example graded, according to the intensity and proximity to a specific trauma focal point. In some embodiments, a focal point is defined by number of emotional terms used to describe it or the behavior of the patient when disclosing this part. In some embodiments, when unclear, a confronted question to the patient, for example “please indicate what is the most distressing part of your memory”, is used to define the trauma focal point.

According to some embodiments, data is collected by using questions, for example guiding questions, in order to characterize emotional, sensory and physical aspects of the experiences (e.g. how did you feel, was your feeling different than usual? what did you see/hear, what did you think, what did you do?). In some embodiments, the clinician receives contextual information, for example about location, other people and time experience. In some embodiments, the information received during the interview is edited into one or more scripts having a duration in a range of 20 seconds-10 minutes, for example 20 seconds-3 minutes, 1 minutes-7 minutes, 5 minutes-10 minutes or any intermediate, smaller or larger range of values. In some embodiments, if the script comprises an audio script, then the audio script is delivered to the trainee in a second person voice. In some embodiments, each or at least some NF training sessions include debriefing following each cycle in the clinic, in order to guide each patient in finding the most useful techniques in order to promote brain modulation.

According to some embodiments, the feedback delivered to the subject is modulated according to the subject success in modulating the activity of the brain region. In some embodiments, the modulated feedback is delivered as a modulated exposure narrative, for example as described above. In some embodiments, during a “rest period” of less than 5 minutes, for example less than 3 minutes, less than 1 minute, less than 30 minutes or any intermediate, smaller or larger value, calculation of an EFP value, for example each trainee mean EFP value during rest and/or a standard deviation (STD) across this average is performed.

According to some embodiments, the feedback, for example an auditory feedback comprising a short auditory neutral context feedback, for example a feedback not specific to a trauma of the trainee, of less than 10 minutes, for example less than 5 minutes, less than 3 minutes or any intermediate, smaller or larger value. In some embodiments, the neutral context feedback comprises a jazz music piece. Alternatively, the short auditory feedback comprises a trauma specific context delivered during exposure sessions. In some embodiments, an increase or decrease in a value of a statistical parameter calculated for a measured EFP signal, for example a change of one STD in amyg-EFP value (either up or down), causes a respective change in the loudness of the auditory feedback, for example a change of 10 dB. In some embodiments, after each NF period the STD is reset in accordance to the target signal values recorded during the last NF period and so on.

According to some embodiments, the EFP-NF which comprises an exposure to a trauma context, trauma content and/or trauma trigger, directly targets brain mechanism while provoking mental processing that is involved in the consolidation/extinction of the traumatic memory and experience. Without being bound by any theory, memories are re-consolidated each time they are retrieved. Accordingly, re consolidation opens a window of opportunity to rewrite emotional memories and require the involved circuitry. That is, old traumatic experiences can be updated and recontextualized during the reconsolidation window (Nader et al., 2000, Dudai et al., 2006, Schiller et al., 2010, Kandler et al., 2014). Therefore, re consolidation while manipulating the relevant circuit, enables non-invasive intervention that not only alter the experience of the traumatic memory but also modify its underlying neural mechanism.

A potential advantage of the EFP-NF training combining exposure to content of the traumatic event while training to volitionally down-regulate amygdala activity, could have a beneficial effect on the sensitivity of the neural system for cued provoked symptoms in PTSD. Exposure to a trauma specific challenge that is combined with the feedback itself in the EFP-NF training program, activates and modulates (online) the brain processes that are coding the traumatic experience. Exposure to a recorded narrative of the trauma is an individual and challenging context that is set to activate the neural processes and networks that are used to code and retain and consolidate the memory of the traumatic event. Training participants to modulate limbic activity while these neural processes and networks are activated could hopefully alter its operation and drive it in a positive direction.

An additional potential advantage of the EFP-NF, is that it is performed in a controlled, gradual pace, with varied level of difficulty that minimizes patient discomfort and subsequent dropout. Therefore, context-related NF could also be recruited to constructing a therapeutic design which includes several difficulty levels built into it. As the trainee manages to regulate successfully in a simple, non-aversive feedback environment, the feedback itself could become more and more related to the patient's pathology context and more emotionally evoking, creating a gradient challenge across the treatment period.

According to some embodiments, the EFP-NF is applied to different context-specific disorders, for example Obsessive Compulsive Disorders (OCD), specific phobia and/or social anxiety. In some embodiments, for OCD, the feedback, for example a positive feedback could be related to the content of the obsessions and/or compulsion, for example, a dirty room that becomes clean, disorganized room that become organized. In some embodiments, for example for Specific phobia from height; the elevator is going down or the doors closed faster, or a frightening object or insect transformed into a pleasant one. In some embodiments, for example for social anxiety, the trainee is required to regulate while performing a task in front of other people and their verbal/facial/gesture feedback about the performance changes gradually from neutral/ambiguous to positive (smile, nice words, leaning towards the trainee with interest).

According to some embodiments, context is used to test an acquired skill. In some embodiments, it is used after learning to challenge the difficulty for example; executive function task for ADHD, memory task for mild cognitive impairment, pain induction for fibromyalgia, hedonic cue for depressive patients, social interaction for borderline personality disorder or related disorders (e.g. Post menstrual dysphoric disorder.

An aspect of some embodiments relates to providing online and continuous feedback to a subject while the subject is exposed to challenge, for example a stress related challenge. In some embodiments, the feedback is provided as the subject negotiates the challenge. In some embodiments, the challenge comprises a non-specific stress challenge. Alternatively, the challenge comprises a stress disorder specific stress challenge. In some embodiments, the feedback is provided by modifying the challenge.

An aspect of some embodiments relates to exposing a subject to a challenge, for example a stress-related challenge during a NF procedure while the subject is not confined by an imaging system. In some embodiments, the subject is not confined, for example surrounded by the imaging system, for example an MRI or an fMRI device. In some embodiments, a supervisor is located near the subject during the exposure and the NF, for example at a distance of up to 10 meters from the subject, for example to allow fast access to the subject in case that the exposure is too intensive.

An aspect of some embodiments relates to providing feedback to a subject by modifying a stress-related challenge delivered to the subject. In some embodiments, the feedback is provided according to an activation level of one or more brain regions affected by the challenge. In some embodiments, the feedback is provided according to an ability of the subject to modify the activation of the one or more brain regions. In some embodiments, if the subject is successful in down regulating the activity of the one or more brain regions then the challenge intensity or severity is lowered. In some embodiments, if the subject is not successful in down regulating the activity of the one or more brain regions, then the challenge intensity or severity remains the same. In some embodiments, the feedback is delivered continuously and/or online.

An aspect of some embodiments relates to teaching a subject to perform at least one exercise, for example a physical or a mental exercise, shown to modulate an activity of at least one stress-related brain region in said subject in a timed relation with an exposure to a trauma or a stress-disorder-trigger. In some embodiments, the at least one exercise is selected and/or developed during an EFP-NF training, in which the ability of the subject to modulate an activation of the at least one stress-related brain region is monitored by measuring at least one electrophysiological parameter, for example EEG signals indicating an activation level of the specific at least one stress-related brain region.

According to some embodiments, the subject performs the at least one exercise after the completion of the EFP-NF training, for example a week, 14 days, a month, 6 months or any intermediate, shorter or longer time period from the completion of the EFP-NF training. In some embodiments, the subject performs the at least one exercise outside the clinic, for example when the subject is at his home, at his workplace or traveling. In some embodiments, the subject performs the at least one exercise while getting feedback from a mobile system that measures at least one physiological parameter indicating an activity level of at least one stress disorder-related brain region, for example an EEG signal. Alternatively, the subject performs the at least one exercise without receiving a feedback regarding the activity level of the at least one stress disorder-related brain region.

According to some exemplary embodiments, the subject performs the at least one exercise while being exposed to a stress-related or a trauma-related interface personalized to the subject. In some embodiments, the interface is delivered to the subject by a user interface of a mobile device, for example a display and/or a speaker of the mobile device. Alternatively or additionally, the subject performs the exercise when receiving an alert indication prior to an appearance of a stress disorder related trigger or a trauma-related trigger, for example an alert indication delivered by a media broadcasting organization or channel.

An aspect of some embodiments relates to safety features while exposing a subject to a stress-related challenge during NF training. In some embodiments, the subject is calmed down, for example by a drug and/or exercises if an effect of the exposure is too intensive. Alternatively or additionally, the NF training is stopped or modified.

An aspect of some embodiments related to delivering a NF training, for example an EFP-NF training while exposing the trainee to a stress disorder-related challenge designed according to at least one impaired neurobehavioral process in the trainee, for example a subject diagnosed with a stress disorder. In some embodiments, the at least one impaired neurobehavioral process is identified based on an assessment of the trainee, for example assessment of symptoms of the stress disorder.

According to some embodiments, at least one of the NF training goals comprises one or more of reduction in the expression level of at least one symptom of the stress disorder and shifting the trainee from the impaired neurobehavioral process towards a less impaired different neurobehavioral process or a non-impaired neurobehavioral process. Alternatively or additionally, the at least one of the NF training goals comprises reducing the level of impairment of the impaired neurobehavioral process in the trainee following and/or during the NF training.

According to some embodiments, the at least one impaired neurobehavioral process in a stress disorder, for example PTSD, comprises one or more of threat detection, emotion regulation, fear extinction, reward consumption, saliency homeostasis, episodic memory encoding and reinstatement, executive function. In some embodiments, the NF training trains a subject diagnosed with a stress disorder to modulate an activation level of at least one brain region, for example a brain region of the limbic system, a deep brain region located under the cortex layer of the brain, and/or a neural circuit related to the at least one impaired neurobehavioral process.

According to some embodiments, an impaired threat detection process involves one or more of the anterior Insula, the ventromedial prefrontal cortex (vmPFC), the periaqueductal gray (PAG), and the locus coeruleus (LC), and the Amygdala. In some embodiments, the NF training is used to teach a trainee to specifically modulate an activation level of one or more of the brain regions involved in the impaired threat detection process. In some embodiments, the trainee modulates an activation level of the at least one brain region while being exposed to an interface personalized based on the impaired threat detection process in the subject.

According to some embodiments, an impaired emotion regulation process involves one or more of the medial prefrontal cortex (mPFC), the dorsolateral prefrontal cortex (dlPFC), the dorsomedial prefrontal cortex (dmPFC), and the Amygdala. In some embodiments, the NF training is used to teach a trainee to specifically modulate an activation level of one or more of the brain regions involved in the impaired emotion regulation process. In some embodiments, the trainee modulates an activation level of the at least one brain region while being exposed to an interface personalized based on the impaired emotion regulation process in the subject.

According to some embodiments, an impaired fear extinction process involves one or more of the dorsal anterior cingulate cortex (dACC), the vmPFC, the Hippocampus, and the Amygdala. In some embodiments, the NF training is used to teach a trainee to specifically modulate an activation level of one or more of the brain regions involved in the impaired fear extinction process. In some embodiments, the trainee modulates an activation level of the at least one brain region while being exposed to an interface personalized based on the impaired fear extinction process in the subject.

According to some embodiments, the NF treatment of stress-disorder patients, for example PTSD patients in enhanced by implementing a Process-Based approach to the design and practice of the NF training, for example an EFP-NF training. In some embodiments, multiple stress-disorder processes, for example PTSD stress-disorder processes are activated via a context based interface during the neuromodulation of related networks. In some embodiments, various combinations of impaired processes are likely to interact as they give rise to the clinical phenomenology.

An aspect of some embodiments relates to treating depression in patients diagnosed with a stress disorder using NF-training, for example EFP-NF training. In some embodiments, the EFP-NF training is used to teach the patient to modulate, for example to actively modulate an activity of at least one specific brain region, for example at least one specific brain region which is related to the stress disorder. In some embodiments, modulation of the activity of the at least one specific brain region reduces the expression of depression symptoms in the patient.

According to some embodiments, at least one parameter of the NF training is adjusted in order to treat depression in the stress disorder diagnosed patient. In some embodiments, the at least one parameter comprises an interface, for example a challenge, delivered to the patient, and/or at least one exercise performed by the patient, for example a physical exercise and/or a mental exercise. In some embodiments, the NF-training is adjusted to treat depression in a stress disorder diagnosed patient treated with a bioactive compound for reducing depression symptoms.

According to some embodiments, the depression is evaluated following the NF training. In some embodiments, the expression level of at least one depression symptom is evaluated following the NF training. In some embodiments, the NF training is modified based on the evaluation results, for example instructions of a different exercise or a modified exercise are delivered to the patient if the modulation of the at least one brain region is not sufficient or is not is a desired direction and/or if the reduction in depression levels following the NF training is not sufficient.

According to some embodiments, an existing drug regime of at least one bioactive compound for treating depression is modified or a new drug regime is initiated following the NF training, for example based on the results of the NF training. In some embodiments, a dosage of the at least one bioactive compound is reduced if the modulation of the activity of the at least one specific brain region is in a desired level and/or if the NF training was effective in reducing at least one symptom of depression in the patient. A potential advantage of reducing a dosage of the at least one bioactive dosage or the prescribing a new bioactive compound with a low dosage may be to reduce side effects in the patient caused by the bioactive compound.

According to some embodiments, a challenge, for example an interface, presented to PTSD patients with depression comprise at least one of environment or protocol (instructions) that guide or stimulate recall of positive autobiographic memories, reward consumption paradigm (getting a reward for more effort), hedonic music.

An aspect of some embodiments relates to treating anxiety in patients diagnosed with a stress disorder using NF-training, for example EFP-NF training. In some embodiments, the EFP-NF training is used to teach the patient to modulate, for example to actively modulate an activity of at least one specific brain region, for example at least one specific brain region which is related to the stress disorder. In some embodiments, modulation of the activity of the at least one specific brain region reduces the expression of anxiety symptoms in the patient.

According to some embodiments, at least one parameter of the NF training is adjusted in order to treat anxiety in the stress disorder diagnosed patient. In some embodiments, the at least one parameter comprises an interface delivered to the patient, and/or at least one exercise performed by the patient, for example a physical exercise and/or a mental exercise. In some embodiments, the NF-training is adjusted to treat anxiety in a stress disorder diagnosed patient treated with a bioactive compound for reducing anxiety symptoms.

According to some embodiments, the anxiety is evaluated following the NF training. In some embodiments, the expression level of at least one anxiety symptom is evaluated following the NF training. In some embodiments, the NF training is modified based on the evaluation results, for example instructions of a different exercise or a modified exercise are delivered to the patient if the modulation of the at least one brain region is not sufficient or is not is a desired direction and/or if the reduction in anxiety levels following the NF training is not sufficient.

According to some embodiments, an existing drug regime of at least one bioactive compound for treating anxiety is modified or a new drug regime is initiated following the NF training, for example based on the results of the NF training. In some embodiments, a dosage of the at least one bioactive compound is reduced if the modulation of the activity of the at least one specific brain region is in a desired level and/or if the NF training was effective in reducing at least one symptom of anxiety in the patient. A potential advantage of reducing a dosage of the at least one bioactive dosage or the prescribing of a new bioactive compound with a low dosage may be to reduce side effects in the patient caused by the bioactive compound.

An aspect of some embodiments relates to delivering an indication to a stress disorder patient to modulate at least one specific brain region related to a trigger of said stress disorder, for example a trigger of at least one symptom of said stress disorder in the patient, prior to an expected exposure to a stress disorder trigger. In some embodiments, the patient receives an indication, for example a human detectable indication to perform at least one exercise selected to modulate an activation of at least one specific brain region in the subject, prior to an exposure to a stress disorder trigger. In some embodiments, the patient receives the indication, optionally automatically, when an alert of an expected trauma trigger is identified.

According to some embodiments, a device, for example a personal assisting device located in the home of the patient, on the patient, and/or near the patient, for example at a distance of up to 20 meters from the patient, for example up to 10 meters, up to 5 meters, up to 2 meters or any intermediate, smaller or larger distance from the patient, identifies at least one alert signal for an expected exposure to a trigger of the stress disorder of the patient. In some embodiments, a control circuitry of the device identifies the alert signal by receiving a signal from a microphone, for example a microphone of the device, or directly from the alert signal source, for example a media broadcasting device generating the alert signal.

According to some embodiments, the devise selects at least one exercise of a list of exercises, according to the identified alert signal. In some embodiments, the list of exercises is stored in a memory of the device. Alternatively, the list of exercises is stored in a remote memory storage, for example a cloud storage, or a remote server. In some embodiments, the device selects the at least one exercise according to at least one of, the alert signal, for example according to the expected trigger of the stress disorder, according to the stress disorder, the at least one brain region that needs to be affected by the at least one exercise, the time the patient has until the expected exposure to the stress disorder trigger, and information on the patient, for example clinical and/or personal information. In some embodiments, the device delivers a human detectable indication to the patient to perform the at least one selected exercise. In some embodiments, the human detectable indication, for example an audio and/or a visual indication, comprises instructions how to perform the at least one selected exercise.

According to some embodiments, the device monitors the activity of at least one specific brain region of the patient, for example in conjunction, for example before, during and/or following, with the performance of the at least one exercise and/or with the exposure to the at least one stress disorder trigger. In some embodiments, the device, for example a control circuitry of the device, monitors the activity of the at least one specific brain region by receiving at least one electrical signal for example electrical signals from the patient, for example electrical signals generated by the brain of the patient. In some embodiments, the electrical signals comprise EEG signals. In some embodiments, the control circuitry of the device receives the EEG signals from at least one EEG electrode attached to the patient. In some embodiments, the device is wirelessly connected to the at least one EEG electrode.

According to some embodiments, the control circuitry of the device processes the at least one electrical signal to identify a relation between an activity signature of the at least one specific brain region, for example an activity signature also termed herein as an EEG electrical fingerprint (EEG-EFP), and at least a portion of the at least one electrical signal. In some embodiments, the processing of the received electrical signals is performed in the device by the control circuitry. In some embodiments, a memory of the device comprises at least one EEG-EFP, or a plurality of EEG-EFPs, which are optionally personalized to the patient, and indicate an activation level of at least one specific brain region in the patient. Alternatively, the at least one EEG-EFP or the plurality of EEG-EFPs, optionally personalized to the patient, are stored in a remote server, for example a remote server of a cloud storage. In some embodiments, the control circuitry of the device transmits the received electrical signals to the remote server and the identified relation between the at least a portion of the received electrical signals and the at least one stored EEG-EFP is performed in the remote server, for example using at least one algorithm or at least one lookup table stored in the remote server.

According to some embodiments, the device, for example a control circuitry of the device delivers a human detectable indication to the patient according to the activation level of the at least one specific brain region. In some embodiments, the device delivers the human detectable indication according to a success of the patient to modulate an activation of the at least one brain region is a desired direction, for example upregulate or downregulate, and/or according to a modulation level, for example based on a score indicating a level of modulation. In some embodiments, the device, for example a control circuitry of the device changes the instructions delivered to the patient, and/or changes an exercise delivered to the patient, based on the activation level of the at least one specific brain region and/o a success of the patient in modulating the activity of the at least one specific brain region using an exercise.

According to some embodiments of the invention, a feedback delivered to a subject diagnosed with a stress disorder, for example a patient and/or a trainee of the NF training, comprises information regarding a direction of modulation of at least one specific brain region, for example downregulation or upregulation, and/or a level, for example a score, indicating a measured degree of modulation.

According to some embodiments of the invention, the feedback regarding an activation level of the at least one specific brain region and/or a success of the patient in modulating the activity level of the at least one specific brain region, is delivered online while the patient is exposed to the challenge. In some embodiments, a control circuitry of a device that is used to deliver the NF training or to monitor an activation level of the at least one brain region, continuously and repeatedly receives electrical signals, for example electrical signals generated by the brain of the patient, identifies a relation between at least a portion of the received electrical signals and a stored signature, for example an EEG-EFP signature, and delivers a feedback to the patient. In some embodiments, the control circuitry receives the at least one electrical signal, determines an activation level of at least one specific brain region based on the signals, and generates a feedback to patient regarding the determined activation level in less than 30 seconds, for example less than 15 seconds, less than 10 seconds, less than 5 seconds, less than 2 seconds or any intermediate, shorter or longer time period.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Introduction

Exposure to aversive and potentially traumatic events is prevalent in modern life. One of the common psychopathology following traumatic exposure is Post-Traumatic Stress Disorder (PTSD), a chronic mental illness, with lifetime prevalence ranging from 1.3 to 12.2% and debilitating prognosis. Functional neuroimaging studies point to limbic dysregulation as a possible neural mechanism in PTSD, optionally manifested by hyperactivation of the amygdala and/or dorsal anterior cingulate cortex (dACC), alongside hypoactivation of the ventro-medial prefrontal cortex (vmPFC) and/or inferior frontal gyrus (IFG).

The amygdala is involved, for example, in emotional processing including initiation and regulation of stress response and is also instrumental in effective emotion regulation. It is therefore reasonable to assume that amygdala functionality could be an effective target for brain-guided intervention through Neurofeedback (NF). According to some exemplary embodiments, NF learning is based on self-modulation of brain activity, guided optionally, by contingent reinforcing feedback that reflects success in modulating a specific neural signal.

According to some exemplary embodiments, a personalized process-based EFP-NF intervention, using individually-tailored trauma-related feedback content is delivered to subjects diagnosed with a stress disorder, for example PTSD. In some embodiments, the EFP-NF intervention modulated the amygdala activity, which is a neural node for PTSD, and is optionally coupled with exposure to personalized trauma-narrative as a feedback interface. In some embodiments, the EFP-NF intervention allows, for example to customize self-neuromodulation of a stress-related abnormal process and therefore increase treatment effectiveness.

### Exemplary Patient Treatment by a NF Training Process

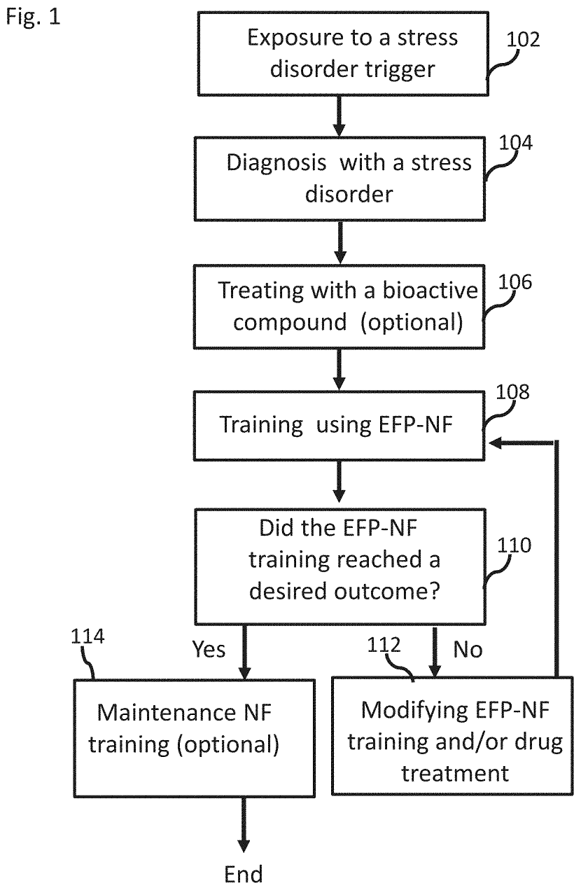

According to some exemplary embodiments, a subject diagnosed with a stress disorder, for example PTSD, is trained to control activation of one or more brain regions related to the stress disorder. In some embodiments, the subject is trained to downregulate activation of one or more brain regions that are activated by a memory of a trauma related to the stress disorder, for example a traumatic event or a trauma-related context. In some embodiments, the NF training, for example an EFP-NF training comprises training while exposure to content related to the trauma. In some embodiments, the subject is trained in order, for example, to reduce stress-related symptoms such as avoidance from trauma cues, hyper arousal, intrusion of thoughts and sensations, alterations in emotional experiences like anhedonia, re-experiencing of distressing memories, dissociative states, generalized avoidance from trauma related cues, dysphoria and/or anhedonia, anger bursts. Reference is now made to FIG. 1 depicting a process for reducing stress-related symptoms from a patient perspective, according to some exemplary embodiments of the invention.

According to some exemplary embodiments, a subject is exposed to a stress disorder trigger, for example to a trigger of a PTSD, at 102. In some embodiments, a PTSD trigger comprises a traumatic environment and/or a traumatic interaction. In some embodiments, the PTSD trigger comprises a transient trigger that lasts for up to 24 hours, for example up to 12 hours, up to 1 hour, or any intermediate, smaller or larger time duration. In some embodiments, the trigger comprises a continuous trigger that lasts for more than 24 hours, for example more than 2 days, more than 7 days or any intermediate, smaller or larger time duration.

According to some exemplary embodiments, the subject is diagnosed with a stress disorder, for example PTSD, at 104. In some embodiments, the subject is diagnosed after a time period of at least 1 day from the exposure to the stress trigger, for example after a time period of at least 1 week, after a time period of 6 months and/or after a time period of several years from the exposure to the stress trigger, or any intermediate, shorter or longer time period. In some embodiments, the subject is diagnosed based on one or more stress-related symptoms. In some embodiments, the subject is diagnosed by a psychiatrist or any other clinician.

According to some exemplary embodiments, the subject, for example, a patient is optionally treated with a bioactive compound at 106. In some embodiments, the bioactive compound comprises one or more drugs and/or pharmaceutical substances. In some embodiments, the bioactive compound is directed to treat the stress-related symptoms. Alternatively or additionally, the bioactive compound is directed to affect activation of one or more brain regions and/or neural circuits, for example brain regions or neural circuits related to the stress disorder, for example stress disorder memory. In some embodiments, the bioactive compound comprises one or more of a Selective Serotonin Reuptake Inhibitor (SSRI), new generation of antiepileptic drugs that are used as mood stabilizers; e.g. Topamax, Bupropion, Ketamine, Cannabis.

According to some embodiments, the drug is taken by the subject prior to or during the NF training, for example to enhance the effect of the NF training. Alternatively or additionally, the drug is taken to protect the subject during the NF training, for example from the effects of an exposure to stress-related challenges.

According to some exemplary embodiments, the patient is trained by the EFP-NF training procedure at 108. In some embodiments, the patient is trained while receiving the bioactive compound. In some embodiments, the EFP-NF training procedure is personalized for a specific patient, for example to the specific stress disorder and/or to a specific trauma caused the stress disorder. Alternatively or additionally, the EFP-NF training procedure is modified according to a specific bioactive compound taken by the patient.

According to some embodiments, the patient, for example a trainee is trained using the EFP-NF to control the activation levels of one or more brain regions related to a stress disorder and/or to a trauma, for example, one or more brain regions related to a memory of the trauma. In some embodiments, the patient is trained to downregulate activation of the one or more brain regions, for example the amygdala. Optionally, the patient is trained to downregulate activation levels of the amygdala during an exposure, for example gradual exposure, to content and/or context related to the trauma, for example a specific trauma that affected the trainee.

According to some embodiments, the ability of the EFP-NF training to reach a desired outcome, for example to reduce stress-related symptoms or to reach a desired activation level of one or more brain regions is evaluated at 110. In some embodiments, the evaluation comprises an interview with the patient after a selected period of time following the EFP-NF, for example after at least one day, for example after a day, after a week, after a month or any intermediate, shorter or longer time period. Alternatively or additionally, the evaluation comprises recording values of one or more clinical parameters, for example heart rate, blood pressure or any other clinical parameter related to stress and/or anxiety. In some embodiments, the evaluation comprises recording of EEG signals and/or fMRI signals following the EFP-NF training. Optionally, the EEG signals and/or fMRI signals are recorded in a time relationship to exposure to a trauma-related content or context, for example during the exposure and/or following the exposure.

According to some exemplary embodiments, if the EFP-NFP training did not reach a desired outcome, then at least one parameter of the EFP-NF training is modified at 122. In some embodiments, the at least one parameter comprises the number of sessions, content of an interface delivered to the trainee during the training, duration of each session, an EFP used during the EFP-NF training. Optionally, a bioactive compound taken by the trainee is replaced to a different bioactive compound. In some embodiments, the trainee repeats the EFP-NF training using the modified EFP-NF protocol at 108.

According to some exemplary embodiments, if the EFP-NF training reached the desired goal, then the trainee is invited to maintenance NF sessions at 114. In some embodiments, the maintenance sessions are configured to maintain the ability of the trainee to affect the activation of the one or more brain regions, for example using the skills the trainee acquired during the NF training at 108. In some embodiments, the NF maintenance sessions are performed a week, a month, a year or any intermediate, smaller or larger time period following the NF training. In some embodiments, the maintenance training is based on recordings of EEG signals or recordings of one or more clinical parameter values indirectly affected by the activation of the one or more brain regions. Optionally, the maintenance NF comprises an EFP of the selected one or more brain regions.

According to some exemplary embodiments, the maintenance sessions are performed by a subject that underwent the EFP-NF training session. In some embodiments, the maintenance sessions are performed by the subject himself, for example in his home or in any other location away from a clinic. In some embodiments, the maintenance sessions are performed by exposing the subject to at least one trauma-trigger, for example at least one trauma-trigger personalized to the trainee. In some embodiments, the personalized trauma trigger is based on a personalized trauma-related scenario presented to the subject during the EFP-NF training.

According to some exemplary embodiments, the subject applies at least one exercise, for example a physical or a mental exercise in a timed relation with the exposure to the personalized trauma trigger, for example before, during and/or after the exposure. In some embodiments, the at least one exercise is applied by the subject in response to an appearance of a trauma-trigger warning, and/or prior to an expected trauma-trigger. In some embodiments, the subject performs applies the at least one exercise in an absence of feedback.

### Exemplary EFP-NF Procedure

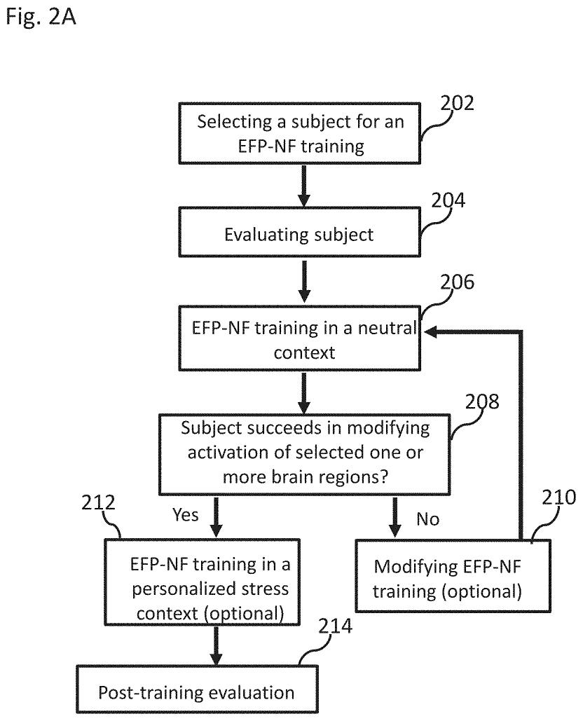

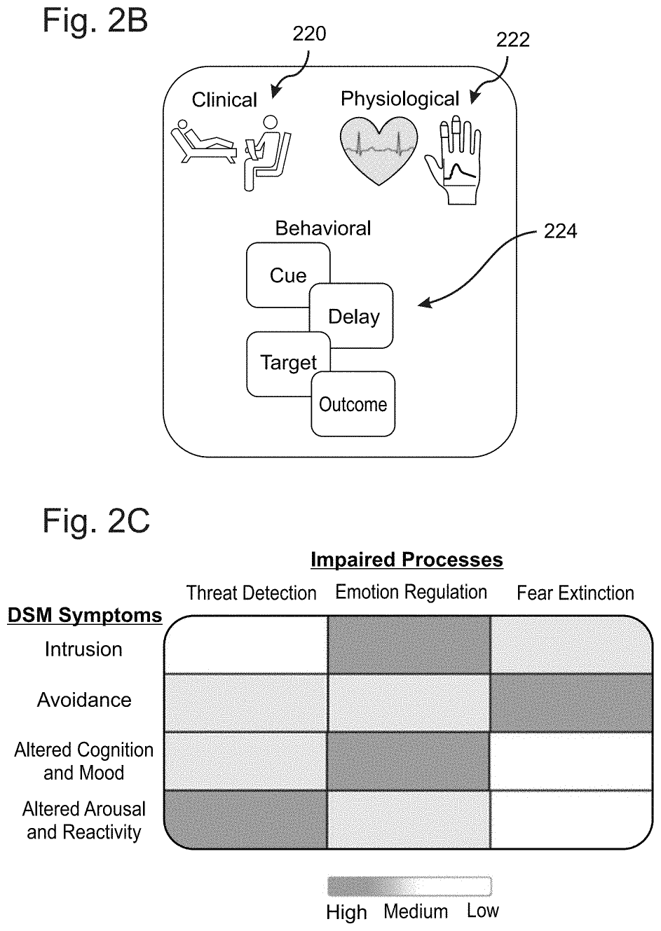

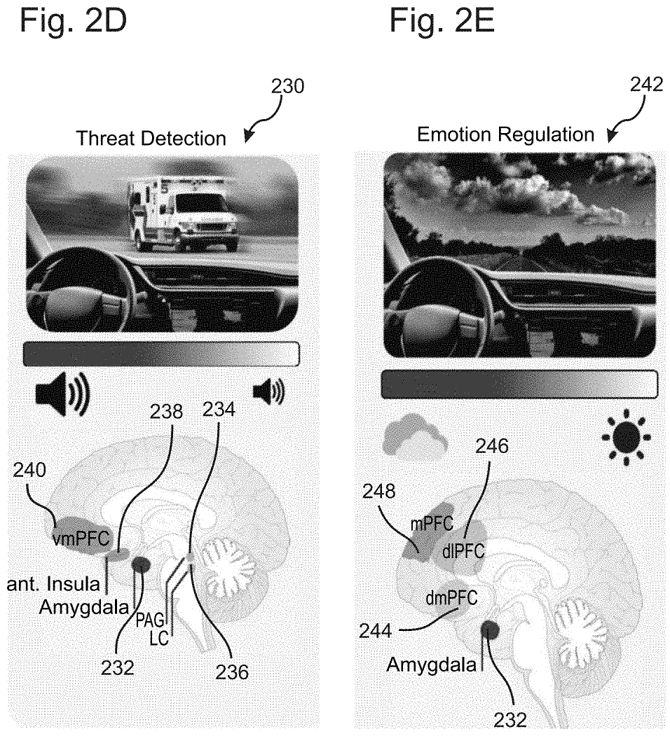

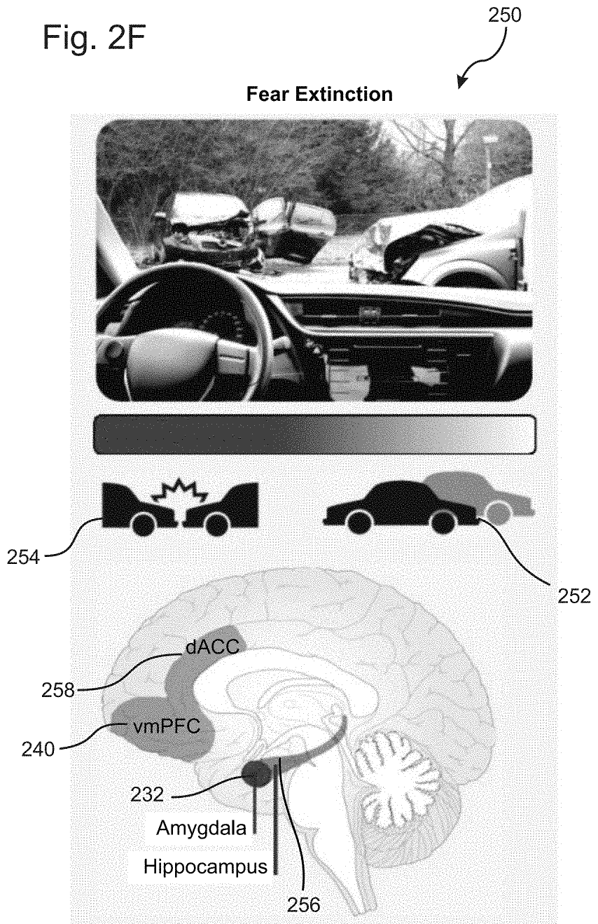

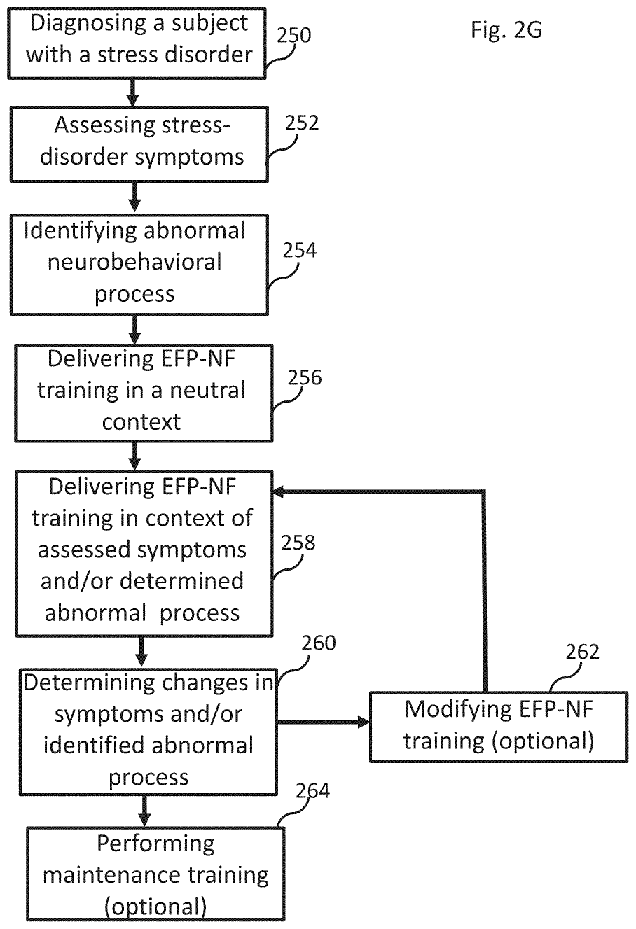

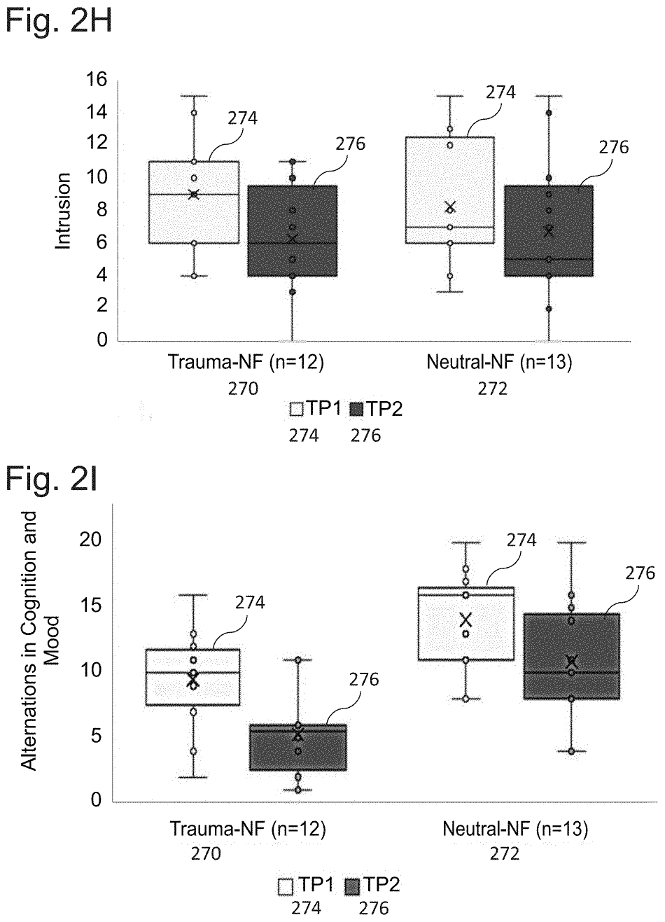

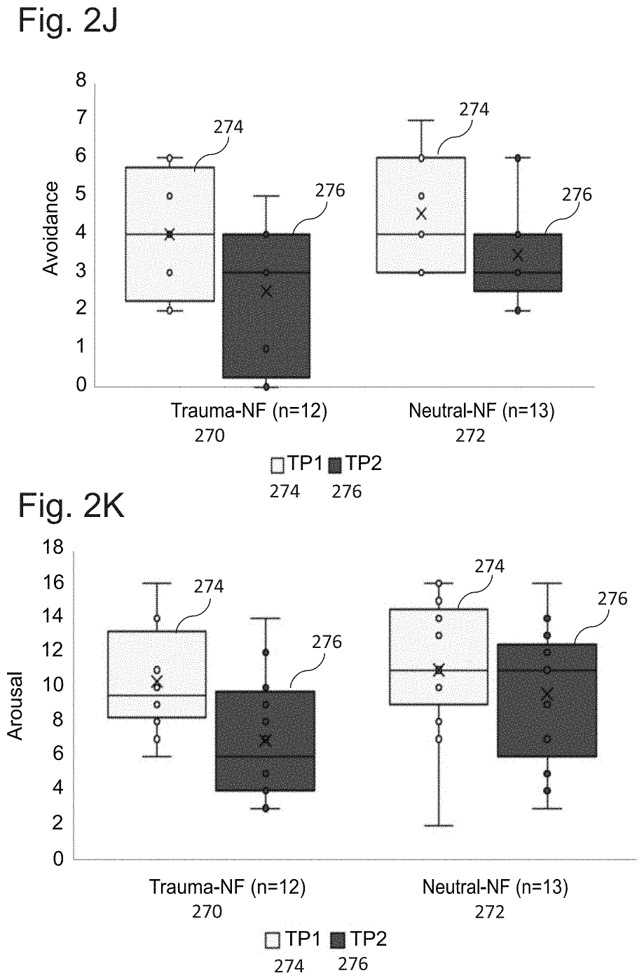

Reference is now made to FIG. 2, depicting an EFP-NF training process according to some exemplary embodiments of the invention.

According to some exemplary embodiments, a subject is selected for an EFP-NF training at 202. In some embodiments, the subject, for example a subject diagnosed with a stress disorder, for example PTSD, is selected by a clinician prior to the EFP-NF training. In some embodiments, the subject is selected based on the stress disorder type, time that passed from a stress disorder trigger or a traumatic event, clinical history, medication history.

According to some exemplary embodiments, the subject is evaluated by a clinician prior to the EFP-NF training at 204. In some embodiments, the subject is evaluated, for example to personalize the EFP-NF training to a specific subject. In some embodiments, the clinician collects information from the subject, for example in an interview about the trauma, for example about the trauma trigger and/or the traumatic event that caused the stress disorder.

According to some exemplary embodiments, the subject is trained using the EFP-NF in a neutral context at 206. In some embodiments, a neutral context is a context that is non-specific to a trauma of the specific subject. In some embodiments, in a neutral context EFP-NF training procedure, the subject learn how to modulate the activation of one or more brain regions, for example the amygdala while reacting to non-specific stress triggers, for example loud noise. In some embodiments, during the neural context EFP-NF the subject is trained to perform one or more exercises, for example physical or mental exercises to modulate, for example to downregulate, amygdala activity.

According to some exemplary embodiments, a success of the subject, for example a trainee to reach a desired activation level of the one or more brain regions is evaluated at 208. In some embodiments, a success of the trainee to downregulate an activity level of the amygdala while reacting to the non-specific stress triggers is evaluated at 208.

According to some exemplary embodiments, if the trainee did not succeed in reaching a desired activation level by the EFP-NF, then optionally, at least one parameter of the neutral context EFP-NF is modified at 210. In some embodiments, the at least one parameter comprises training duration, duration of each session, type and/or content of the non-specific stress trigger, the way the non-specific stress trigger is delivered to the trainee, strategy to find an exercise that allows efficient activity modulation in the specific subject.

According to some exemplary embodiments, if the trainee demonstrated a success in reaching a desired activation by the neutral context EFP-NF, then the trainee initiates an EFP-NF training protocol in a personalized stress context at 212. In some embodiments, the trainee performs the exercises learned in the neutral context EFP-NF while reacting to content relating to his personal trauma. Optionally, the trauma-specific content is delivered to the trainee gradually. In some embodiments, the trauma-specific content is delivered to the trainee under a supervision of a supervisor, for example a clinician. In some embodiments, the trainee and/or the supervisor receive online and optionally continues feedback on the success of the trainee to reach a desired activity level of the one or more brain regions while reacting to the trauma-specific content.

According to some exemplary embodiments, a post-training evaluation is performed at 212. In some embodiments, the post-training evaluation evaluates a progress of the trainee compared to a baseline or previous activation measurements of the one or more brain regions. In some embodiments, the post-training evaluation is performed by measuring fMRI signals, EEG signals or values of at least one clinical parameter, for example a clinical parameter associated with activation levels of the one or more brain regions.

### Exemplary Symptoms or Process-Based NF for PTSD

According to some exemplary embodiments, a subject is diagnosed with PTSD using a traditional definition of PTSD according to DSM-5, which consists of five main criteria: (A) exposure to a traumatic experience (defined as death, threatened death, actual or threatened serious injury, or actual or threatened sexual violence), and experiencing subsequent suffering for more than a month from the time of the event (B) intrusive re-experiencing of memories and feelings of the traumatic event (C) avoidance of cues that remind the individual of the event (D) altered cognition, including deficient memory of the event, poor cognitive processing of emotions (i.e. alexithymia), and mood dysregulation related to loss of pleasure, poor emotion regulation and emotional outbursts, and (E) general hyper arousal and vigilance. However, in some embodiments, this categorization results in clinical heterogeneity that can lead to poorly tailored management for individual patients.

According to some exemplary embodiments, in addition or as an alternative to the PTSD diagnosis according to DSM-5, various underlying impairments in neurobehavioral processes, for example, fear learning and extinction, threat detection, emotion regulation and others are identified. In some embodiments, these impairments in neurobehavioral processes are related to amygdala activity.

According to some exemplary embodiments, when considering brain mechanisms, such a conceptualization emphasizes a pivotal role of the Amygdala in different aspects of PTSD, with respect to its relation with other limbic, salience and prefrontal regions, for example as described in Fenster R J et al. 2018. For example, altered fear learning, impaired extinction and safety signal processing are important for PTSD symptomatology, but also depend on the Amygdala functionality of its subnuclei and various cell types, as well as on maladaptive top-down cortical inhibition. Similarly, emotion dysregulation, resulting in intense emotional reactivity, irritability, and impulsivity are known to be underlined by a lack of cortical control over Amygdala reactivity, specifically manifested by impaired connectivity patterns between the Amygdala and the vmPFC and dlPFC, for example as described in Etkin A. et al., 2015. In addition, maladjusted threat detection may give rise to increased attention and reactivity to threatening or salient stimuli and is accompanied by hypervigilance and related aggressive behavior, mediated by the Amygdala as well as cortical regions such as the insula, vmPFC, dACC, as well as brain stem areas such as the PAG and locus coeruleus.

Reference is now made to FIGS. 2B-2F, depicting stages in a personalized process-based NF training, according to some exemplary embodiments of the invention.

According to some exemplary embodiments, a PTSD patient undergoes assessment of impaired processes related to PTSD. In some embodiments, the impaired process characterization is guided by assumed neuroscientific mechanism that underlie the main symptom clusters in PTSD. In some embodiments, for example as shown in FIG. 2B, the assessment comprises clinical assessment 220, for example based on an interview and/or filling at least one questionnaire. Additionally or alternatively, the assessment comprises physiological assessment 222, for example measurements of at least one physiological parameter, for example heart rate, blood pressure, electromyography and/or skin conductivity. Additionally or alternatively, the assessment comprises behavioral assessment 224, for example by monitoring or visualizing the subject response, for example to external perturbation which are optionally trauma-specific for the subject.

According to some exemplary embodiments, for example as shown in FIG. 2C, the expression level of PTSD symptoms, for example PTSD symptoms according to the DSM, in the subject is determined based on the assessment performed in FIG. 2B. In some embodiments, the PTSD symptoms comprise intrusion, avoidance, altered cognition and mood, and altered arousal and reactivity.

According to some exemplary embodiments, based on behavioral and physiological assessments it is possible to establish an individually-tailored process-related characterization of PTSD patients. In some embodiments, for example as shown in FIG. 2C, different processes are related to PTSD abnormalities, for example threat detection, emotion regulation and fear extinction. In some embodiments, the processes are measured by administering behavioral tasks, for example predictable and unpredictable shock task for threat detection (Schmitz A. et, al., 2012), emotional regulation task for emotion regulation (Shafir R. et, al., 2015), and an aversive learning task for fear extinction (Shalev L., et, al., 2018). In some embodiments, PTSD symptom clusters, for example intrusion, avoidance, altered cognition and mood, and altered arousal and reactivity, are depicted, for example as shown in the table shown in FIG. 2C, according to their suggested weights in each of the major dysfunctional processes per patient. In some embodiments, the impaired processes are identified, based on the weight or level of expression of each of the PTSD symptoms. Additionally or alternatively the impaired processes are identified based on the assessment performed in FIG. 2B.

According to some exemplary embodiments, the impaired processes in PTSD, for example threat detection, emotion regulation, and fear extinction are linked to PTSD symptoms, and are optionally related to an increase in PTSD severity.

According to some exemplary embodiments, for example as shown in FIGS. 2D-2F, the dysfunctional processes are used for delivery of individually tailored process based NF. In some embodiments, the dysfunctional processes derived from the initial assessment battery guide the selection of the corresponding intervention interface. In some embodiments, each interface is configured to specifically target an impaired process by provoking activity in a designated brain circuitry including the Amygdala. In some embodiments, self-modulation of the Amygdala in each unique context results in specific modulation patterns of the underlying circuit of interest.

According to some exemplary embodiments, for example as shown in FIG. 2D, in the case of impairment in a threat detection process 230, an interface with threat related cues is delivered to the subject, and the Amygdala activity feedback corresponds, for example to the volume of an ambulance siren. In some embodiments, this process-specific context provokes modulation of threat detection related circuits involved in, for example, at least one of increased attention, reactivity to threatening stimuli and hypervigilance, such as the anterior Insula 238, the ventromedial prefrontal cortex (vmPFC) 240, the periaqueductal gray (PAG) 234 and the locus coeruleus (LC) 236, and the Amygdala 232. In some embodiments, electrical fingerprints (EFP) of the Amygdala, and one or more of these regions are used to determine the activation level of these regions, and to generate and/or modify a feedback to the subject based on the determined activation level.

According to some exemplary embodiments, for example as shown in FIG. 2E, in the case of impairment in an emotion regulation process 242, an interface with emotion related cues is delivered to the subject, and the Amygdala activity feedback corresponds, for example, to a brightness level of the interface. In some embodiments, this process-specific context provokes modulation of emotion regulation related circuits involving at least one of the medial prefrontal cortex (mPFC) 248, the dorsolateral prefrontal cortex (dlPFC) 246, the dorsomedial prefrontal cortex (dmPFC) 244, and the Amygdala 232. In some embodiments, electrical fingerprints (EFP) of the Amygdala, and one or more of these regions are used to determine the activation level of these regions, and to generate and/or modify a feedback to the subject based on the determined activation level.

According to some exemplary embodiments, for example as shown in FIG. 2F, in the case of impairment in a fear extinction process 250, an interface with fear related cues is delivered to the subject, and the Amygdala activity feedback corresponds, for example, to the content of the interface. In some embodiments, this process-specific context provokes modulation of fear extinction related circuits involving at least one of the dorsal anterior cingulate cortex (dACC) 258, the vmPFC 240, the Hippocampus 256, and the Amygdala 232. In some embodiments, electrical fingerprints (EFP) of the Amygdala, and one or more of these regions are used to determine the activation level of these regions, and to generate and/or modify a feedback to the subject based on the determined activation level.

### Exemplary Procedure for Symptoms or Process-Based NF Training

According to some exemplary embodiments, a NF training program for a subject diagnosed with a stress disorder, for example PTSD, is personalized to the specific subject. In some embodiments, the NF training program is personalized, according to at least one of a trigger that caused a trauma in the past, a current trigger that causes at least one stress symptom related to the trauma, at least one stress-disorder symptom, and at least one abnormal process related to the stress disorder. Reference is now made to FIG. 2G, depicting a NF training procedure for a subject diagnosed with a stress disorder, for example PTSD, according to some exemplary embodiments of the invention.

According to some exemplary embodiments, a subject is diagnosed with a stress disorder, at block 250. In some embodiments, the stress disorder comprises PTSD, Acute Stress Disorder, Adjustment disorder, Reactive attachment disorder (RAD), Disinhibited social engagement disorder (DSED), Obsessive compulsive disorder, phobia, general anxiety, social anxiety, borderline personality disorder. In some embodiments, the subject is diagnosed with a stress disorder according to the diagnostic and statistical manual of mental disorders (DMS), for example a stress disorder in the categories “other Trauma and Stressor-Related Disorders” or “unspecified Trauma- and Stressor-Related Disorder” in the DSM, 5^{th} edition.

According to some exemplary embodiments, the subject is diagnosed with the stress disorder based on an observation and/or an interview with a mental health professional, for example a psychiatrist. Alternatively or additionally, the subject is diagnosed with the stress disorder based on measurement results of at least one physiological parameter, for example heart rate, blood pressure and/or skin electrical conductivity.

According to some exemplary embodiments, symptoms of the stress-disorder, for example are assessed at block 252. In some embodiments, the symptom assessment is performed as described for example in FIG. 2B. In some embodiments, the symptoms are assessed based on an observation and/or an interview with a mental health professional, for example a psychiatrist. Alternatively or additionally, the symptoms are assessed based on measurement results of at least one physiological parameter, for example heart rate, blood pressure and/or skin electrical conductivity. Additionally or alternatively, the symptoms are assessed using an assessment questionnaire, for example a Clinician-Administered PTSD Scale (CAPS), for example CAPS-DSM-5 (CAPS-5) or variations thereof. In some embodiments, the symptoms are assessed based on subclasses of the CAPS-5. In some embodiments, the symptoms comprise one or more of Intrusion, Avoidance, Altered Cognition and Mood, and Altered Arousal and Reactivity. In some embodiments, the assessment comprises determining a strength, for example an expression level of each of the symptoms in the specific subject.

According to some exemplary embodiments, abnormal neurobehavioral processes, for example abnormal stress-related neurobehavioral processes are identified at block 254. In some embodiments, the abnormal neurobehavioral processes are identified based on the assessment of the stress-disorder symptoms performed at block 252, for example based on an expression level of the symptoms in a specific subject. Alternatively or additionally, the abnormal neurobehavioral processes are identified based on the subject diagnosis performed at block 250.

According to some exemplary embodiments, the abnormal neurobehavioral processes comprise PTSD-related abnormal neurobehavioral processes, for example an impaired threat detection process, an impaired emotion regulation process, and/or an impaired fear extinction process.

According to some exemplary embodiments, a NF training is delivered to the diagnosed subject, for example a PTSD patient, at block 256. In some embodiments, the delivered NF training is an electrical finger print (EFP)-NF training. In some embodiments, the delivered EFP-NF training at block 256 is in a neutral context, and not in a context related to the trauma, a trigger of the trauma, a PTSD-related symptom or a PTSD-related abnormal neurobehavioral process.

According to some exemplary embodiments, in the neutral context EFP-NF, the patient is instructed to perform at least one exercise, for example a physical or a mental exercise while interacting, for example sensing, watching and/or listening to an interface delivered to him by tactile sensation, audio and/or video signals, for example using virtual reality or on a display. In some embodiments, the interface is a neutral interface, for example non-related to the PTSD.

According to some exemplary embodiments, performing the at least one exercise modulates an activation level of at least one neural circuitry, or at least one brain region, for example at least one brain region related to the limbic system. In some embodiments, the at least one brain region related to the limbic system comprises the Amygdala. In some embodiments, the subject receives a feedback according to the activity level of the at least one neural circuitry or the at least one brain region. In some embodiments, the feedback is delivered by modulating the interface.

According to some exemplary embodiments, the subject performs the EFP-NF in a neutral context, for example to find the exercise, for example the physical and/or mental exercise that show high efficiency relative to other exercises in modulation of the activity of the at least one brain region in the subject. Additionally, performing the EFP-NF in a neutral context also allows, for example, to determine whether the subject qualifies for moving to EFP-NF sessions in a trauma context.

According to some exemplary embodiments, an NF training, for example an EFP-NF training is delivered in a trauma context, at block 258. In some embodiments, the trauma context is personalized to the trainee. In some embodiments, the trauma context comprises EFP-NF training in context of PTSD symptoms in the trainee, for example PTSD symptoms assessed at block 252. Alternatively or additionally, the trauma context comprises EFP-NF training in context of PTSD-related abnormal neurobehavioral processes in the trainee, for example abnormal processes identified at block 254.

According to some exemplary embodiments, changes in the assessed symptoms and/or identified abnormal processes, are determined at block 260. In some embodiments, the changes in the assessed symptoms and/or identified abnormal processes are determined during a training session, for example in the beginning and/or in an end of a training session, for example while the trainee is in the clinic. Alternatively or additionally, changes in the assessed symptoms and/or identified abnormal processes are determined between training sessions, for example while the trainee is in his home and not in the clinic or in a clinic.

According to some exemplary embodiments, if the determined changes are desired changes, for example changes that reduce the expression of symptoms and/or modified the abnormal process in a desired level, then the NF training is stopped. Alternatively, if the determined changes are not desired changes, for example changes that did not reduce the expression of symptoms and/or modified the abnormal process in a desired level, the n the EFP-NF is continued or repeated. Alternatively, the EFP-NF training is stopped.

According to some exemplary embodiments, the EFP-NF is modified at block 262. In some embodiments, the at least one exercise performed by the trainee is modified. Alternatively or additionally, the trauma-related interface delivered to the trainee is modified.

According to some exemplary embodiments, after a successful completion of the EFP-NF, for example after reaching the desired goals of the training, for example after reaching the desired changes, the trainee perform at least one additional maintenance training session at block 264. In some embodiments, the maintenance training session is performed at least 1 week, for example at least 2 weeks, at least 1 month, or any intermediate, shorter or longer time duration after completing the EFP-NF training. In some embodiments, the maintenance training session is performed without receiving feedback in relation to the activity level of the at least one brain region and/or neural circuitry. In some embodiments, the maintenance training session is performed while the trainee is not in the clinic. In some embodiments, in the maintenance training session, the trainee perform at least one exercise, for example the exercise the trainee performed during the training session, at block 258. Alternatively, the trainee performs a different exercise, which is optionally selected based on the clinical condition of the trainee and/or PTSD symptom levels.

### Exemplary Changes in Symptoms Following Treatment

According to some exemplary embodiments, changes in stress-related symptoms are monitored following the NF training and/or between training sessions of the NF training, for example as described at block 260 in FIG. 2G. Optionally, based on the changes in the symptoms, changes in stress-related abnormal neurobehavioral processes are determined. Reference is now made to FIGS. 2H-2K depicting changes in stress-related symptoms, for example PTSD-related symptoms, following EFP-NF training, according to some exemplary embodiments of the invention.

According to some exemplary embodiments, for example as shown in FIG. 2H, trainees that were exposed to a Trauma-related interface in a Trauma-NF training 270 exhibited reduction in Intrusion following the training in a TP2 276 (post-training test point), compared to levels in Intrusion prior to the training at TP1 274 (pre-training test point). Intrusion levels also reduced in trainees that performed Neutral-NF training 272 with a neutral interface not related to the Trauma.

According to some exemplary embodiments, for example as shown in FIG. 2I trainees that were exposed to a Trauma-related interface in a Trauma-NF training 270 exhibited reduction in alterations in cognition and mood following the training, in a TP2 276 (post-training test point), compared to levels in alterations in cognition and mood prior to the training at TP1 274 (pre-training test point). Alterations in cognition and mood levels were also reduced in trainees that performed Neutral-NF training 272 with a neutral interface not related to the Trauma, but the reduction was smaller compared to the Trauma-NF training 270 group.

According to some exemplary embodiments, for example as shown in FIG. 2J trainees that were exposed to a Trauma-related interface in a Trauma-NF training 270 exhibited reduction in avoidance levels following the training, in a TP2 276 (post-training test point), compared to levels of avoidance prior to the training at TP1 274 (pre-training test point). Avoidance levels were also reduced in trainees that performed Neutral-NF training 272 with a neutral interface not related to the Trauma, but the reduction was smaller compared to the Trauma-NF training 270 group.

According to some exemplary embodiments, for example as shown in FIG. 2K trainees that were exposed to a Trauma-related interface in a Trauma-NF training 270 exhibited reduction in arousal levels following the training, in a TP2 276 (post-training test point), compared to levels of arousal prior to the training at TP1 274 (pre-training test point). Arousal levels were also reduced in trainees that performed Neutral-NF training 272 with a neutral interface not related to the Trauma, but the reduction was smaller compared to the Trauma-NF training 270 group.

### Exemplary Feedback Delivery

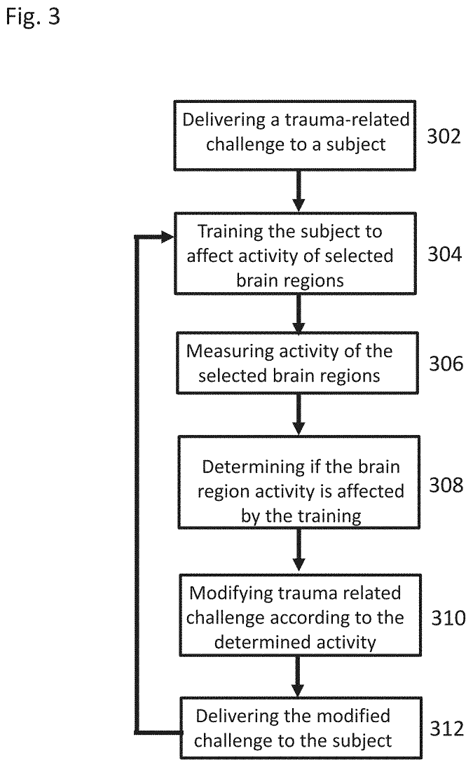

According to some exemplary embodiments, the trainee is exposed to trauma-related content, for example trauma-related content personalized to a specific trauma of the trainee, while training to modulate activation of one or brain regions that are activated by the trauma-related content. In some embodiments, the trainee is exposed to trauma-related content which upregulates the activation levels of the amygdala while training to downregulate the activation levels by one or more exercises, for example mental and/or physical exercises. In some embodiments, a feedback regarding a success of the trainee in modulating the brain activity region is delivered by modifying the trauma-related content. In some embodiments, if the trainee succeeds in downregulating the amygdala activity level, then the intensity of the exposure decreases. In some embodiments, the exposure level of the trainee to the trauma-related content follows a change in the activity level of the amygdala. In some embodiments, if the amygdala activity level decreases, then the exposure level to the trauma content decreases, for example exposure time or exposure content. Reference is now made to FIG. 3, depicting a process for modifying a feedback delivered to a trainee, according to some exemplary embodiments of the invention.

According to some exemplary embodiments, a trauma-related content, for example a trauma-related challenge is delivered to a trainee at 302. In some embodiments, the trauma-related challenge comprises content personalized to a specific trainee, for example based on interviews with a clinician and/or based other information sources. In some embodiments, the trauma-related challenge is delivered by one or more of sound, visual, smell, sensation or any other interface.

According to some exemplary embodiments, a trainee is trained to affect activity of selected one or more brain regions at 304. In some embodiments, the subject is trained in a time relationship to the delivery of the trauma-related challenge, for example during the delivery of the challenge or following the delivery of the challenge. In some embodiments, the training comprises performing predetermined exercises, for example physical and/or mental exercises selected to affect the activation of the one or more brain regions. Alternatively, the subject is encouraged or directed to generate the exercises or to modulate know exercises, for example to fit his own abilities.

According to some exemplary embodiments, an activity level of the one or more brain regions is measured at 306. In some embodiments, the activity level is measured based on EEG signals recorded from the head of the trainee, for example during the EFP-NF training. In some embodiments, the recorded EEG signals are analyzed and an EFP is generated based on the recorded EEG signals. In some embodiments, the generated EFP is related to an activation level of the one or more brain regions.

According to some exemplary embodiments, an effect of the NF training, for example EFP-NF training on the activity of the one or more brain regions is determined at 308. In some embodiments, the effect of the NF is determined based on the generated EFP and a desired EFP, which relates to a desired activity.

According to some exemplary embodiments, the trauma-related challenge delivered to a trainee is modified according to a current activity level of the one or more brain regions at 310. In some embodiments, the trauma-related challenge delivered to the trainee is modified according to a success of a trainee to reach the desired activity level of the one or more brain regions. Alternatively or additionally, the trauma-related challenge is modified according to a change of the EEG signals and/or the extracted EFP signals in a desired direction, for example down or up regulation relative to a baseline or previous signals values. In some embodiments, the modification in the trauma-related challenge is delivered as a feedback of NF training, for example a sensory rewarding feedback of the NF process.

According to some exemplary embodiments, the modified challenge is delivered to the trainee continuously or incrementally. Alternatively or additionally, the feedback is delivered to the trainee online during the NF training, for example in less than 30 seconds from measuring the EEG signals, for example in less than 20 seconds, in less than 10 seconds or any intermediate, shorter or longer time duration.

### Exemplary System for Delivery of NF for Stress Disorders Training

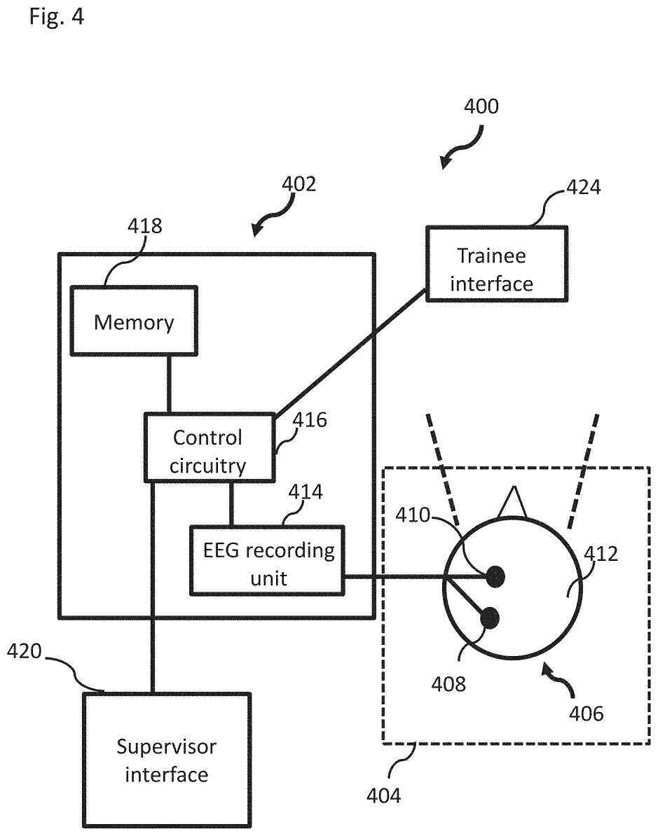

Reference is now made to FIG. 4, depicting a system for delivery of NF, for example EFP-NF for stress disorders training, according to some exemplary embodiments of the invention.

According to some exemplary embodiments, a system, for example system 400 comprises a control unit 402, and an EEG measuring unit 404 electrically connected to the control unit 402. In some embodiments, the EEG measuring unit comprises one or more EEG electrodes, for example EEG electrodes 408 and 410 which are attached to a scalp 412 of a subject 406. In some embodiments, the one or more EEG electrodes are configured to measure EEG signals, for example during the EFP-NF training.

According to some exemplary embodiments, the control unit 402 comprises a control circuitry 416 and an EEG recording unit 414, electrically connected to the control unit 416. In some embodiments, the EEG electrodes, for example EEG electrodes 408 and 410 are electrically connected to the EEG recording unit 414. In some embodiments, the EEG recording unit 414 transmits the recorded EEG signals to the control circuitry 416.

According to some exemplary embodiments, the control unit 402 comprises a memory 418 electrically connected to the control circuitry 416. In some embodiments, the memory 418 stores recorded EEG signals, and stored EFP signals, for example generic EFP signal and/or a personalized EFP signal which relates to a specific activation level of one or more brain regions. In some embodiments, the control circuitry 416 analyzes at least a portion of the recorded EEG signals, for example to generate an EFP which relates to a current activity level of one or more brain regions. In some embodiments, the control circuitry 416 generates the EFP signal using an algorithm stored in the memory 418.

According to some exemplary embodiments, the control circuitry is electrically connected to a trainee interface 424. In some embodiments, the trainee interface 424 comprises an audio interface and/or a display. In some embodiments, during the NF training, the control circuitry delivers a challenge, for example a neutral challenge and/or a trauma-specific challenge to the trainee by the trainee interface. In some embodiments, values of at least one parameter related to the neutral challenge and/or to the trauma-related challenge are stored in the memory 418. In some embodiments, the trainee interface 424 delivers the challenge, by generating audio and/or visual signals. Alternatively or additionally the trainee interface 424 delivers the challenge to the trainee 406 by generating smell.

According to some exemplary embodiments, the control circuitry 416 is electrically connected to a supervisor circuitry 420. In some embodiments, the supervisor interface 420 delivers one or more indications regarding the NF training process to a supervisor, for example a clinician monitoring the NF training. In some embodiments, the supervisor interface is located near the trainee 406, for example in the same room of the trainee and/or at a distance of less than 10 meters from the trainee, for example at a distance of less than 7 meters, less than 5 meters, less than 3 meters or any intermediate, smaller or larger distance from the trainee 406.

According to some exemplary embodiments, the control circuitry is configured to record EEG signals from the one or more EEG electrodes, for example during the delivery of the challenge to the trainee 406. Additionally, the control circuitry is configured to record EEG signals from the one or more EEG electrodes when the trainee performs exercises to modulate the activity of one or more brain regions. In some embodiments, the control circuitry 416 is configured to analyse the recorded EEG signals and to generate an EFP signal which relates to an activity level of the one or more brain regions during the delivery of the challenge and/or following the performed exercises.

According to some exemplary embodiments, the control circuitry 416 is configured to determine whether a generated EFP signal following the performed exercises relates to a desired activity level of the one or more brain regions based on stored EFP signals, optionally using a lookup table and/or an algorithm stored in the memory 418. In some embodiments, the control circuitry is configured to deliver feedback, for example an indication to the trainee 406, optionally by the trainee interface 424. In some embodiments, the feedback relates to a success of the subject 406 in modifying the activity of the one or more brain regions in a desired direction, for example activity upregulation or activity downregulation.

According to some exemplary embodiments, the control circuitry 416 is configured to modulate at least one parameter of the challenge delivered to the trainee 406 according to a success level of the trainee in modulating the activity level of the one or more brain regions and/or according to an activity level of the one or more brain regions. In some embodiments, the control circuitry 416 modulates the challenge delivered to the trainee 406 based on values of one or more parameters of the challenge, for example content of the challenge, challenge type, challenge duration, sound intensity, light intensity, colors, colors intensity, smell intensity, different sound sources volume (human and or objects), appearance of touch (e.g. wind or water), a person avatar in the training interface (with or without), stored in the memory 418.

According to some exemplary embodiments, a supervisor monitors the performance of the trainee 406 by receiving one or more indications to the supervisor interface 420 from the control circuitry 416. In some embodiments, the control unit is configured to measure values at least one physiological parameter of the trainee, for example heart rate, blood pressure, skin conductance, pupil dilation, specific eye movement towards an exposure stimuli, Electromyography (EMG) for muscle tension or reaction time for specific goal reach, or any other physiological parameter that is associated with a physiological response to stress. In some embodiments, the control circuitry 416 is configured to automatically stop the NF training if measurements of the at least one physiological parameter and/or recorded EEG signals indicate that a trainee response to the challenge, for example a neutral challenge or a trauma-related challenge is stronger than a predetermined value stored in the memory 418. Alternatively or additionally, a supervisor using the supervisor interface manually stops the NF training, if measurements of the at least one physiological parameter and/or recorded EEG signals indicate that a trainee response to the challenge, for example a neutral challenge or a trauma-related challenge is stronger than a predetermined value stored in the memory 418.

According to some exemplary embodiments, a user, for example a technician, a nurse or a physician of the system, for example system 400 inserts information and/or characteristics of a stress-disorder patient, for example subject 406 diagnosed with the stress disorder, into a memory, for example memory 418 of the control unit 402. In some embodiments, the user inserts the information via a user interface of the control unit, for example supervisor interface 420. Alternatively, the information and/or characteristics of the patient are delivered by a communication circuitry of the control unit into the memory 418. In some embodiments, the information comprises information regarding a trauma event initially triggered the stress disorder in the patient, at least one symptom of the stress disorder expressed in the patient, and/or at least one impaired neurobehavioral process in the patient which is linked to the stress disorder.

According to some exemplary embodiments, the information inserted into the memory comprises at least one challenge configured to trigger at least one symptom of the stress disorder in the patient. Alternatively or additionally, the information inserted into the memory comprises at least one non-specific challenge, configured not to trigger at least one symptom of the stress disorder in the subject. In some embodiments, a non-specific challenge is configured to affect an activation of at least one brain region in the patient which is also affected by the stress disorder in the patient, without triggering at least one symptom of the stress disorder in the patient.

According to some exemplary embodiments, the control circuitry, for example control circuitry 416 selects a challenge from a list of challenges stored in the memory, to present to the patient. In some embodiments, the control circuitry selects the challenge based on information on the patient stored in the memory, for example information regarding a trauma that triggered the stress disorder, information on at least one trigger of the stress disorder, information on at least one symptom of the stress disorder expressed in the patient and information on at least one impaired neurobehavioral process of the stress disorder in the patient. Alternatively or additionally, the control circuitry selects the challenge based on values or indications thereof of at least one physiological parameter, for example heart rate, blood pressure, skin conductivity, stored in the memory. Alternatively or additionally, the control circuitry selects the challenge based on results of at least one clinical assessment performed to the patient, for example CAPS-5 assessment or variations thereof, depression assessment and/or anxiety assessment.

According to some exemplary embodiments, the control circuitry, for example control circuitry 416 presents, for example by visual and/or audio signals, at least one challenge, selected by a user or aby the control circuitry, to the patient, for example using a trainee interface, for example trainee interface 424. In some embodiments, the control circuitry, for example control circuitry 416, receives at least one electrical signal, for example at least one electrical signal generated by the brain of the patient, in conjunction with the exposure of the patient to the at least one challenge, for example before, during and/or following the exposing. In some embodiments, the received electrical signals are EEG signals. In some embodiments, the control circuitry receives the at least one electrical signal from a recording unit, for example an EEG recording unit 414.

According to some exemplary embodiments, the control circuitry, for example control circuitry 416 monitors an activation level of at least one specific brain region in the brain of the patient, for example a brain region of the limbic system, using the received at least one electrical signal. In some embodiments, the control circuitry processes the received at least one electrical signal to identify at least a portion of the at least one electrical signal that indicates an activity level of the at least one specific brain region, for example as described in in U.S. patent application Ser. No. 13/983,419.

According to some exemplary embodiments, the control circuitry, for example control circuitry 416, identifies a relation between the at least a portion of the received electrical signal to a signature, also termed herein as an EEG electrical fingerprint (EEG-EFP), stored in the memory, for example memory 418. In some embodiments, the signature is an activity level signature of at least one specific brain region, for example the amygdala or any other brain region of the limbic system.

According to some exemplary embodiments, the control circuitry, for example control circuitry 416, monitors an ability of the patient to modulate the activation of the at least one brain region, for example using the received at least one electrical signal. In some embodiments, the control circuitry delivers instructions to said patient to perform at least one exercise, for example a mental or a physical exercise configured to affect the activation of the at least on specific brain region. In some embodiments, the control circuitry selects the exercise from a list of exercises stored in the memory. In some embodiments, the control circuitry selects the exercise based on the patient information stored in the memory, and/or based on the at least one specific brain region, and/or a based on a desired effect on the activity of the at least one specific brain region.

According to some exemplary embodiments, the control circuitry, for example control circuitry 416, determines how to modify, optionally automatically modifies, at least one of the challenge presented to the subject, and/or instructions of the exercise, based on received electrical signals. In some embodiments, the control circuitry modifies the at least one of the challenge presented to the subject, and/or instructions of the exercise based on an activation level of the at least one specific brain region and/or a success of the patient in modulating the activity of the at least one brain region.

### Exemplary Validation Experiment

Various embodiments and aspects of the present invention as delineated hereinabove and as claimed in the claims section below find experimental support in the following validation experiment.

Post-traumatic stress disorder (PTSD) may be characterized by excessive emotion reactivity and diminished emotion regulation. These process abnormalities may correspond to hyperactive amygdala and hypo-active ventro-medial PFC, respectively. Optionally, nonspecific targeting of these processes might explain low treatment efficacy in PTSD. Hence, a new process-oriented neurofeedback (NF) targeting fMRI inspired EEG-finger-print (EFP) of the amygdala (Amyg-EFP) in the context of exposure to individual trauma narrative, is suggested. This intervention may result in symptom reduction and in altering dysfunctional neural processing in PTSD related to emotion reactivity and regulation.

Patients were randomly assigned to either Amyg-EFP-NF with neutral context (n=14), Amyg-EFP-NF with traumatic context (n=13) or a control group (Treatment As Usual, n=13). In both test groups patients received NF training for 15 sessions (20 min each), preceded and followed by clinical evaluation, amygdala-fMRI-NF and emotion reactivity fMRI-task. Evaluation results demonstrated that patients in both test groups demonstrated reduced PTSD symptoms following the intervention, while participants in the control group did not improve (sig interaction group-treatment), with trauma context showing the largest clinical effect. Reduced amygdala BOLD activation following NF treatment in the test groups compared to treatment as usual group, demonstrated target engagement.

The following examples show the role of disorder specific context for impactful NF in PTSD. Further, the results imply that symptom amelioration in PTSD may be associated with modulation of underlying PTSD neural mechanisms.

In the experiment, the feasibility and efficacy of an amygdala targeted NF treatment in reducing PTSD symptoms in chronic PTSD population that has been suffering from PTSD symptoms for more than 14 months, was examined. Targeting chronic patients allows us to examine whether affecting amygdala activity (for example through NF) is an efficient intervention method given that the symptoms and involved brain mechanisms have consolidated to some degree. The experiment allows us to examine the effect of the context of NF training.

The study includes a test group that trains amygdala down regulation following a challenge that is trauma specific. In some embodiments of the invention and in the study, patients in the trauma context group are gradually challenged with content from their traumatic event (in form of an auditory script) and train on down regulation in this challenging context that is assumed to increase amygdala activation.

In some embodiments of the invention and in the study, clinical outcome measures include CAPS, BDI, STAI, ERQ. Neural outcome measures include a resting state functional connectivity sequence, an amygdala reactivity task (Hariri), an emotional interference task (Emotional Stroop) and two short cycles of amygdala rt-fMRI-NF.

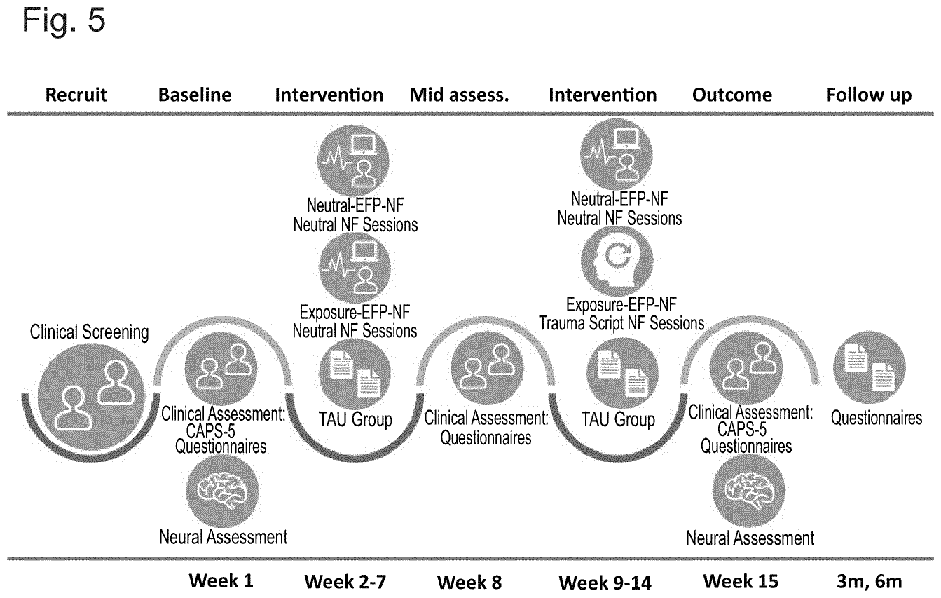

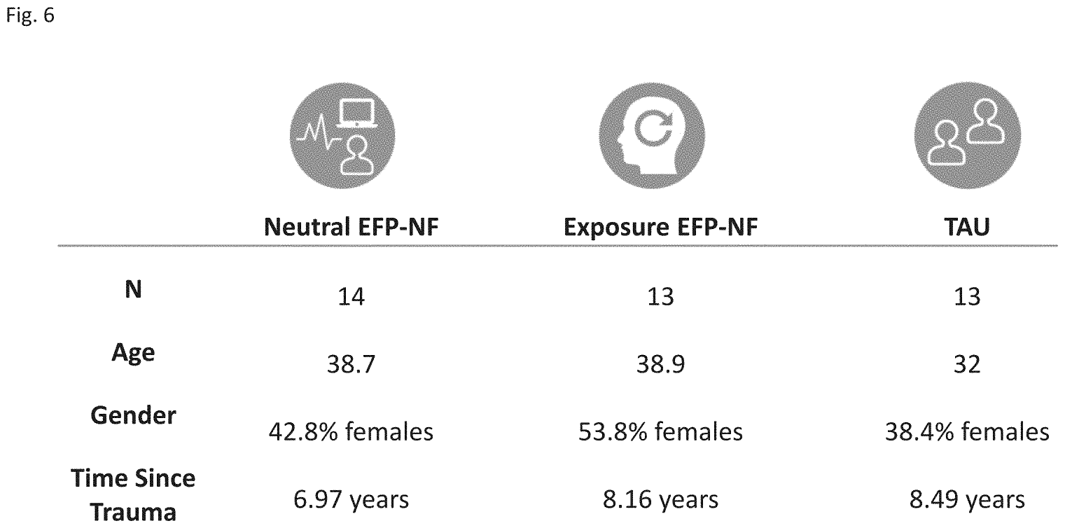

Reference is now made to FIGS. 5 and 6, depicting a general outline of the experiment and the different test groups included in the experiment, respectively.

Participants between the ages of 18-65, who have been exposed to a traumatic event at least 14 months ago were recruited from the trauma clinic at TASMC and from other clinics at the area of Tel Aviv. In some embodiments, subjects exposed to a traumatic event at least 6 months, for example at least 10 months, at least 12 months or any intermediate, smaller or larger time duration, prior to the NF-treatment or an evaluation meeting are selected for the EFP-NF treatment, for example as described at block 202 shown in FIG. 2A. In some embodiments, subjects that were exposed to a traumatic event that lead to a diagnosis of a stress disorder are selected for the EFP-NF treatment.

Inclusion and exclusion: in some embodiments of the invention and in the study participants were screened (and excluded) for the presence of DSM axis-I disorders (using SCID). In some embodiments of the invention and in the study, participants who are exhibiting PTSD symptoms according to CAPS were included. In some embodiments of the invention, the methods that are used for the evaluation of subjects during the experiment are used as part of the subject evaluation at block 204 shown in FIG. 2A. Following evaluation participants were randomly divided into three groups: amy-EFP-NF-neutral group (n=20), amy-EFP-NF-trauma group (n=20) and Treatment as usual (TAU) group (n=20) which will continue on with its other treatment program and will not receive NF training, for example a s shown in FIG. 6.

In the experiment and in some embodiments of the invention, for example at block 204 in FIG. 2A, subject assessment is performed as described in time point 1—pre-treatment: The first time point (TP1) baseline assessments was performed in two to three sessions and included the clinical evaluation, psychological questionnaires and fMRI scan.

The following training phase is an example to the EFP-NF training performed at block 206 in FIG. 2A, in some embodiments of the invention. Training phase: Each participant (in the two test groups) received 15 NF sessions in a duration of 13 weeks (two sessions per week in the first two weeks followed by one weekly session). The number of sessions is similar to the custom duration and or session amounts of common PTSD intervention such as CPT (Resick & Schnicke, 1993) and PE (Rauch et al., 2009) that include exposure. The intervention protocol for the amy-EFP-NF-neutral group included two types of interfaces in an interleaved manner: the first session will use the auditory interface; the second session will use the Waiting Room scenario interface (also described above) and so on until the last session in the intervention period.

In some embodiments of the invention and in the experiment the protocol for the amy-EFP-NF-trauma group was similar in the first session and then gradually included the traumatic challenge NF. In the first phase that included at least 4 sessions, the participants were trained in NF sessions identical to those of the neutral group. As performed for example at block 208 in FIG. 2A, and in the experiment, participants who succeeded in lowering EFP signal during training (compared to baseline) in the 3 out of the last 5 sessions, qualified to continue to the next phase of training. Participants who are not successful continued to train in the neutral context for the remaining sessions.

The following is an example of an EFP-NF training in a personalized stress context, as performed at block 212 in FIG. 2A, in some embodiments of the invention. In the experiment, in the second phase, participants who succeeded as described above, trained in the context of their traumatic story. In the first session, the participants were interviewed about the traumatic event by a trained team member in order to produce a scripted detailed chain of events, including thoughts, feelings, sensations and specific information (day of the week, weather, etc.) in order to create context.

Following the interview which was expected to create an emotional challenge, the participants performed a regular neutral NF training accompanied by one team member. Participants who were successful at down regulation of their EFP signal during this session went on to train with trauma challenge feedback in the following sessions. In some embodiments of the invention and in the experiment, each scripted event was processed to an audio file of at least 30 seconds, for example a three-minute audio file, optionally recorded in a male voice, narrating the event in present-time and second-person (i.e. “you are driving your car . . . ”). In the following sessions of the experiment and in some embodiments of the invention, the feedback indicating the EFP signal was the volume of the trauma-recording. That is, a successful reduction of EFP signal reduced the volume of the trauma recording. Participants that did not succeed in down regulating their EFP signal (with the neutral context) immediately after listening to the trauma-recording tried again in the following NF-N session.

For both groups, each NF practice session consisted of 5 blocks of 3 minutes of NF. Following each block the staff member reviewed with the participant the results of the previous block and asked about the strategies he or she used and their subjective feeling during the block. The last three sessions included two additional “transfer” blocks at the beginning of the session.

During the training phase participants in all groups filled out a bi-weekly symptom monitoring questionnaire that included questions pertaining intrusive thought and feelings, avoidance, cognitive and emotional symptoms, sleep difficulties, and arousal.

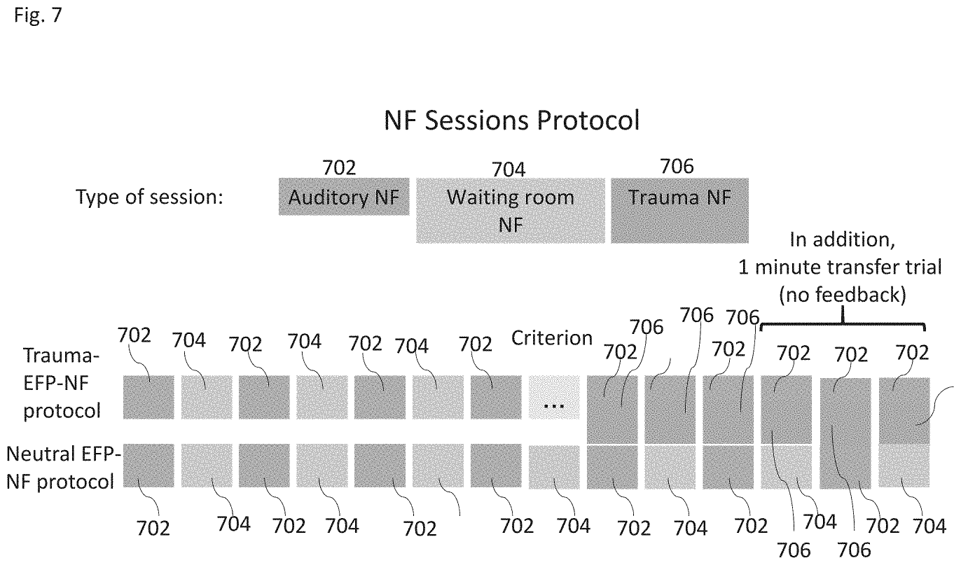

FIG. 7 describes an NF session in a training protocol, according to some exemplary embodiments of the invention and as performed in the experiment, which comprises sessions with auditory NF (blue boxes 702), waiting room NF (green 704) and Trauma NF (purple 706). The figure describes the order of each of the sessions in a Trauma-EFP-NF protocol and in a Neutral EFP-NF protocol.

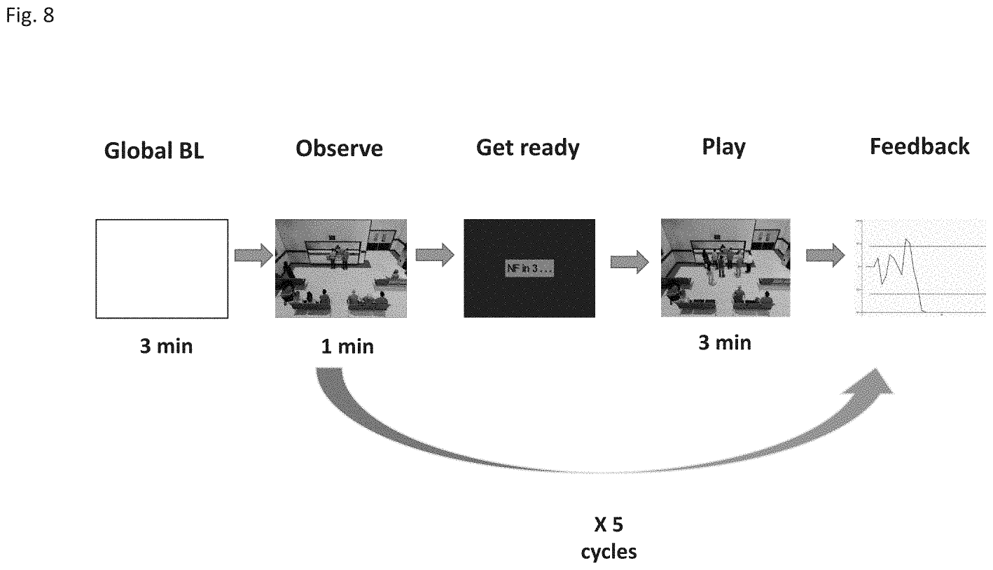

FIG. 8 describes a NF with a multimodal interface: audiovisual animated scenario of a Waiting Room, for example as performed at blocks 108, 206, and 226 in FIGS. 1, 2A, and 2B respectively.

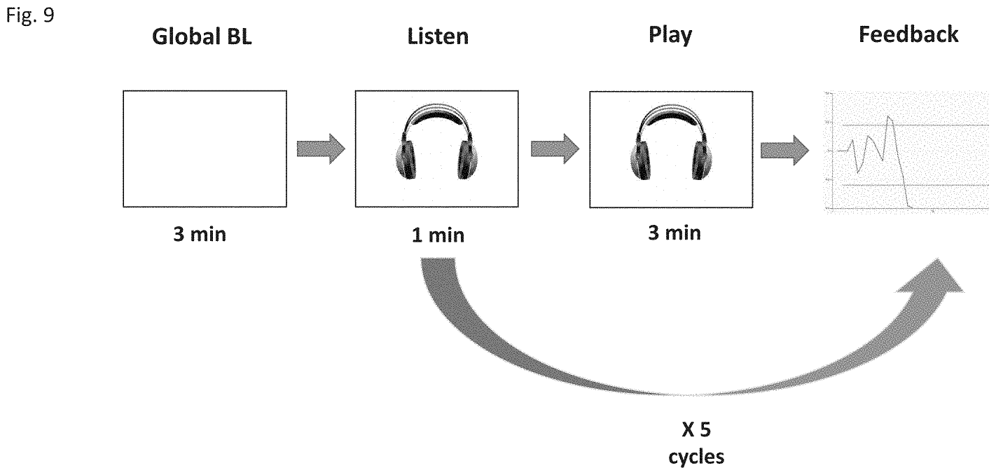

FIG. 9 describes a NF with a single modal auditory interface, for example as performed at blocks 108, 206, and 226 in FIGS. 1, 2A, and 2B respectively.

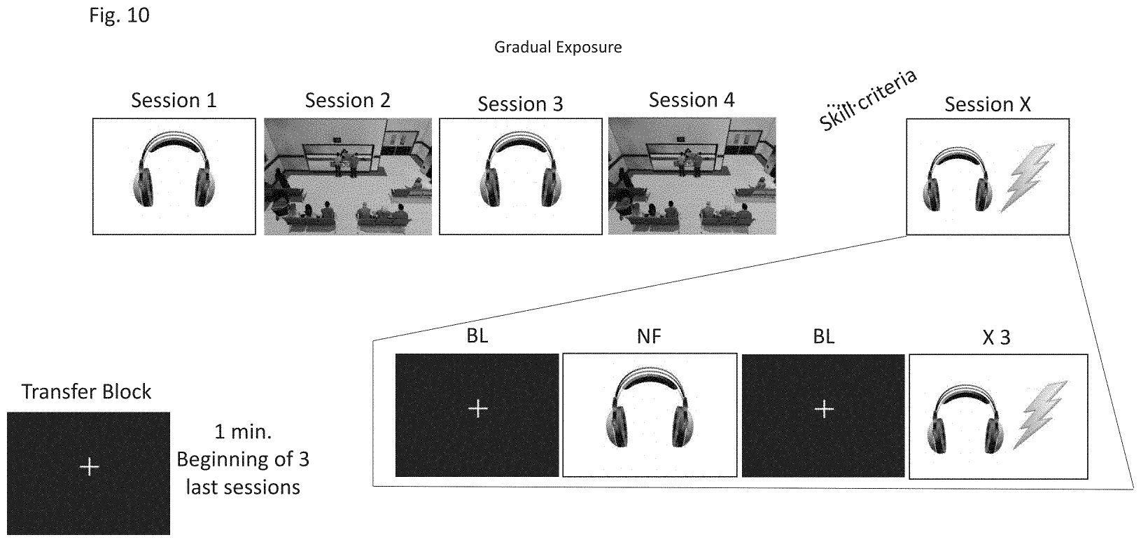

FIG. 10 describes a NF with gradual exposure, for example as performed at blocks 108, 212, and 228 in FIGS. 1, 2A, and 2B respectively.

Experiment Results

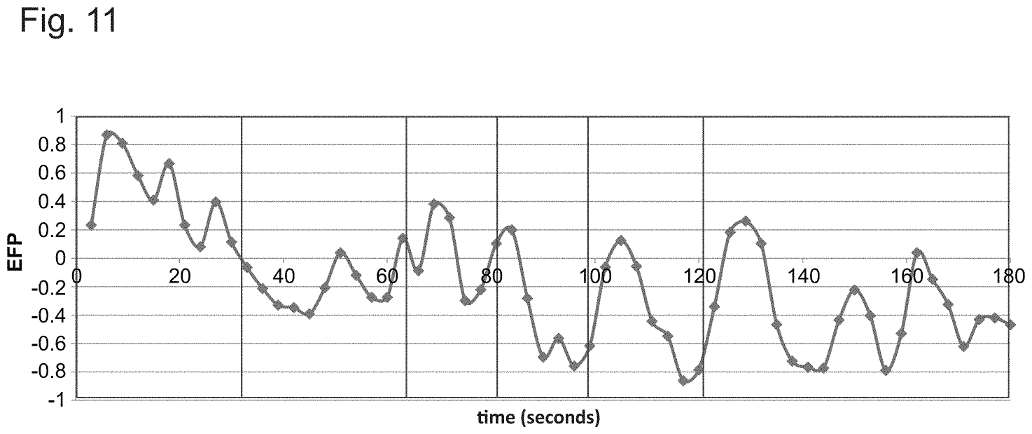

FIG. 11 describes changes in EFP signal in time, while the subject is exposed to a detailed account of the traumatic event. The scenario details contextual information (date, weather, who was there and so on) as well as the emotional, physical and cognitive experience of the patient and the manner in which it changes as the event unfolds.

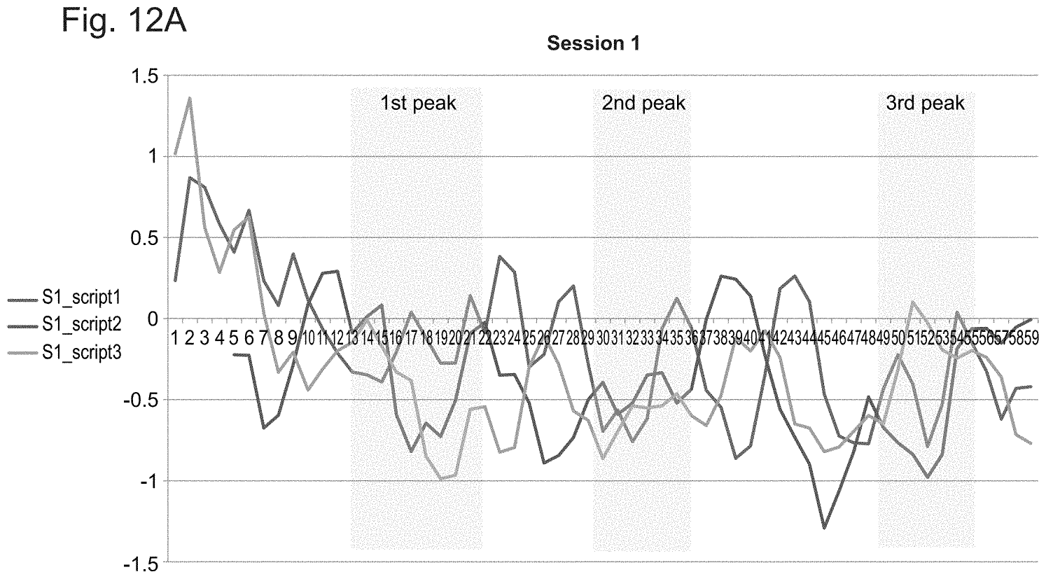

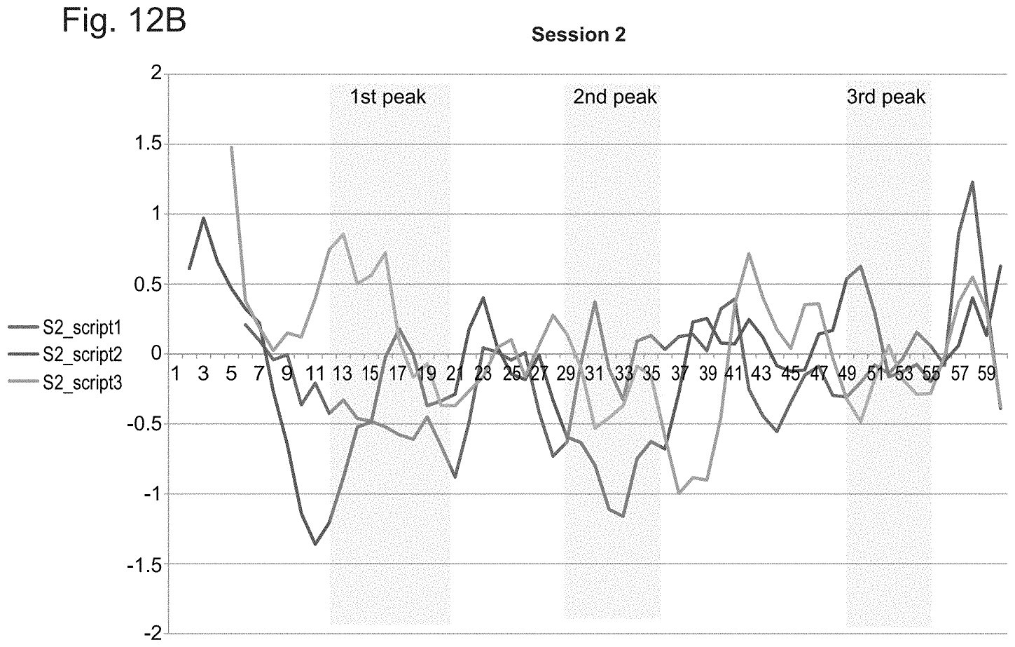

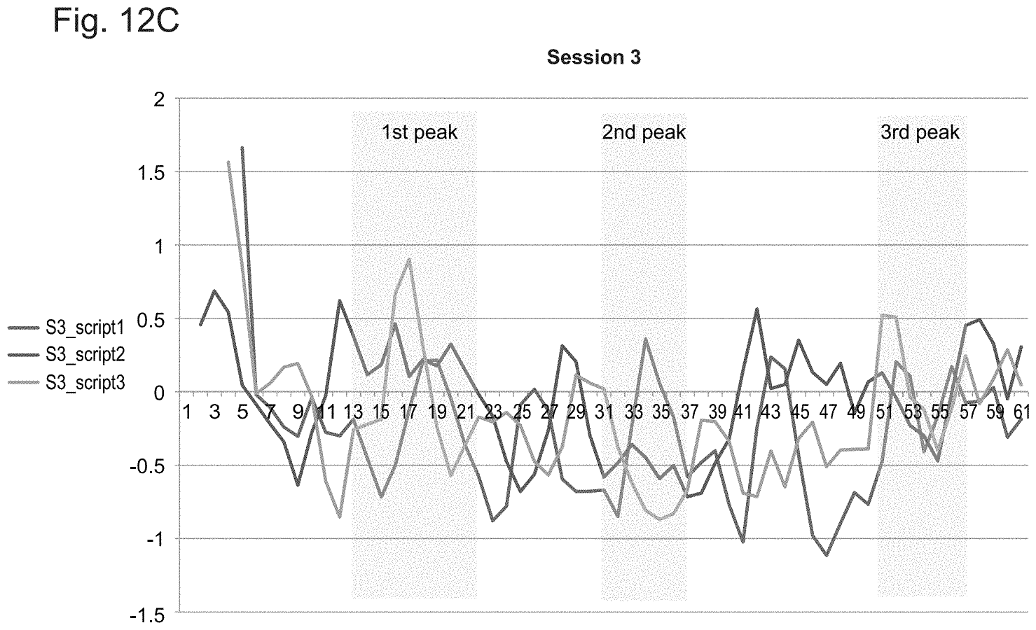

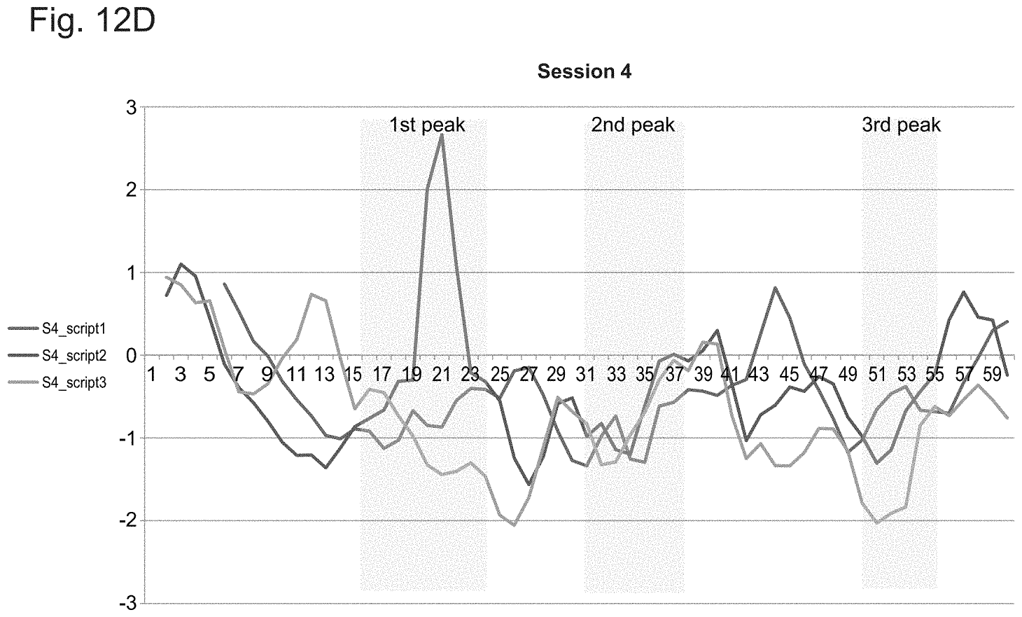

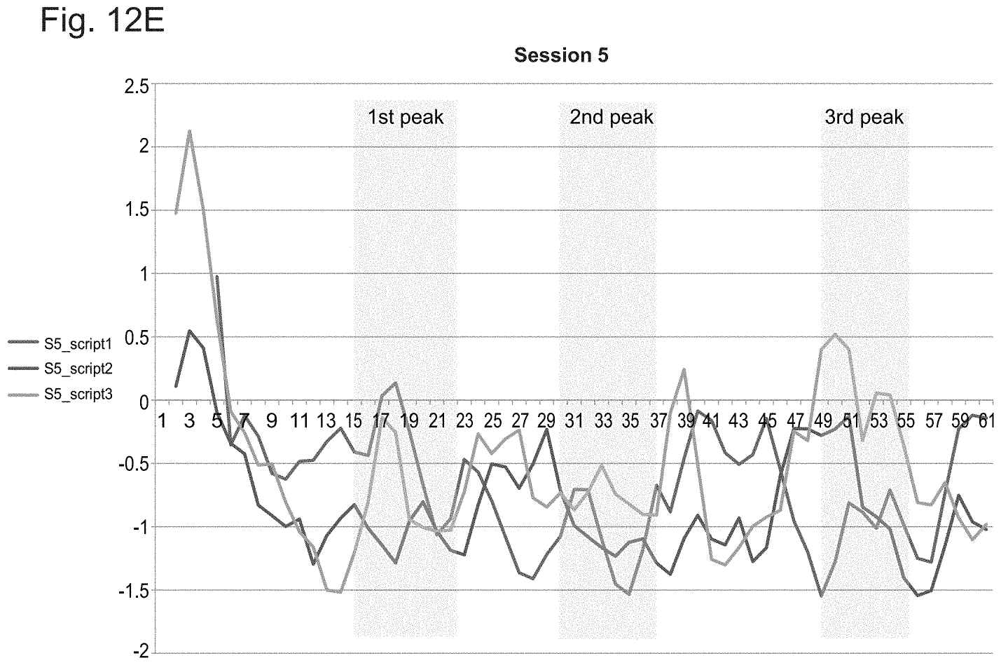

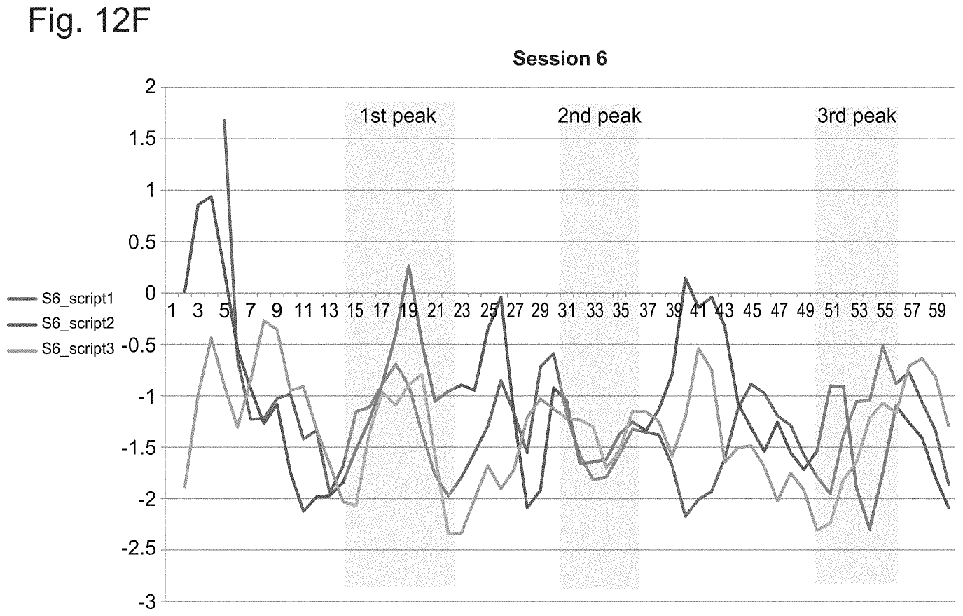

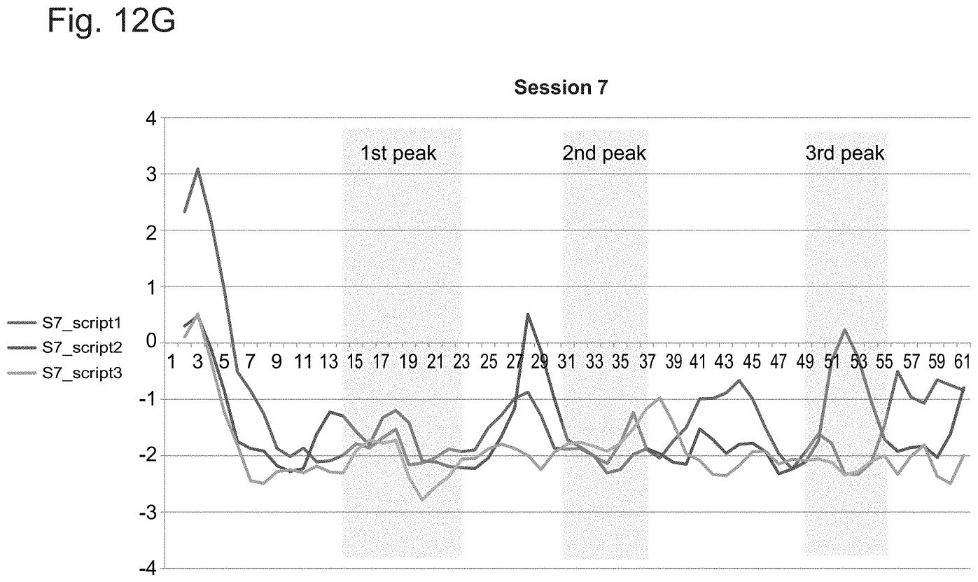

FIGS. 12A-12G demonstrate that a patient is able to down regulate EFP signal more so as the sessions progress and that this down regulation is evident even in the face of intense emotional challenge caused by the “peaks” in the trauma script.

As shown in FIGS. 11 and 12A-12G, a subject modulates an EFP signal while exposed to a scenario having sections that describe a “hot spot”, for example a focal point of a specific trauma (yellow sections).

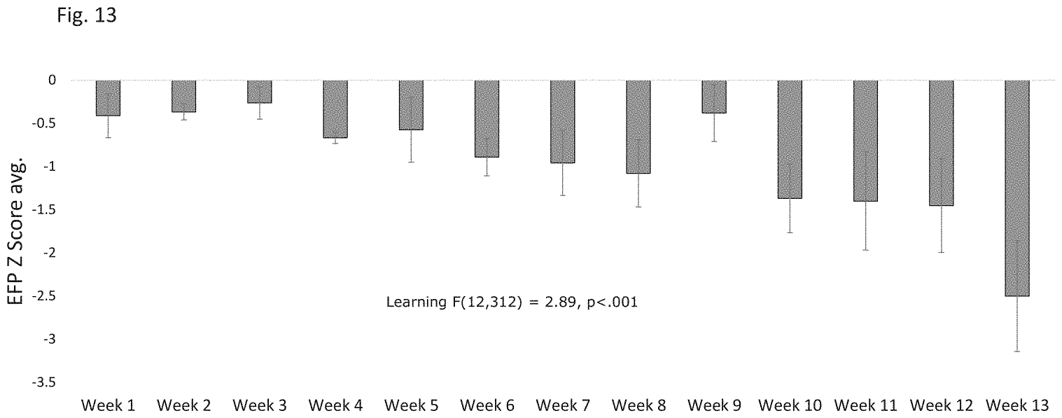

FIG. 13 demonstrates significant learning (i.e. amygdala EFP down regulation) throughout training sessions. Patients were successful at down regulation their amygdala (EFP) activity after 13 weeks of training. As shown in the graph the EFP signal is reduced between week 1 and week 13 (week 1+2—two sessions a week, week 3-13—one session a week, in total: 15 sessions in 13 weeks.

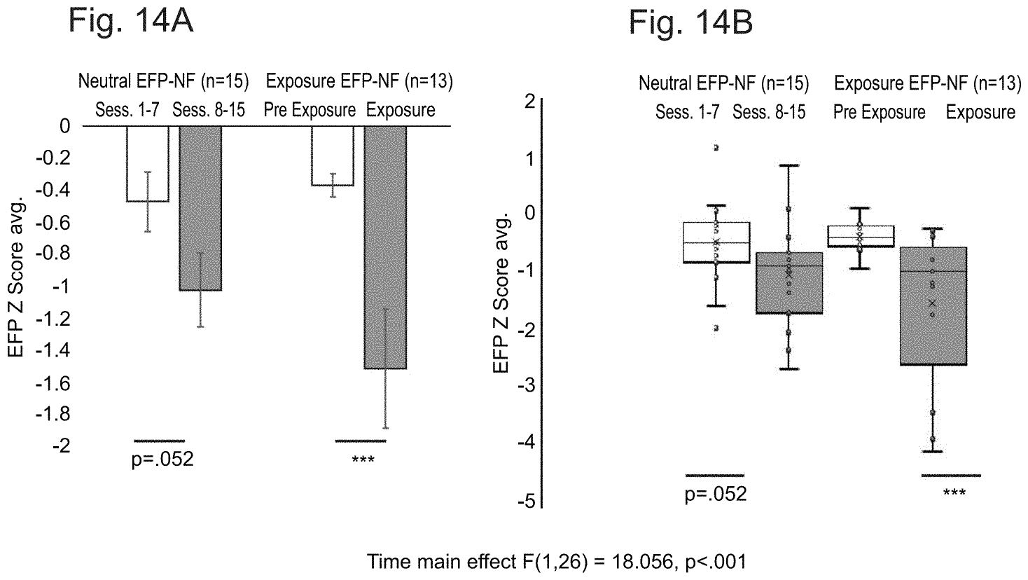

FIGS. 14A and 14B demonstrate NF learning per treatment type, neutral vs exposure). FIG. 14A demonstrates overall means while FIG. 14B shows variability in individual scores. These graphs show that patients that trained in a neutral context were successful at down regulating their EFP signal from the first part of the trial to the second part of the trial. In comparison, patients in the exposure group showed a larger reduction of EFP signal during exposure sessions, compared to neutral context sessions. This figure shows that training EFP in an individual trauma context has an additive effect over neutral context training.

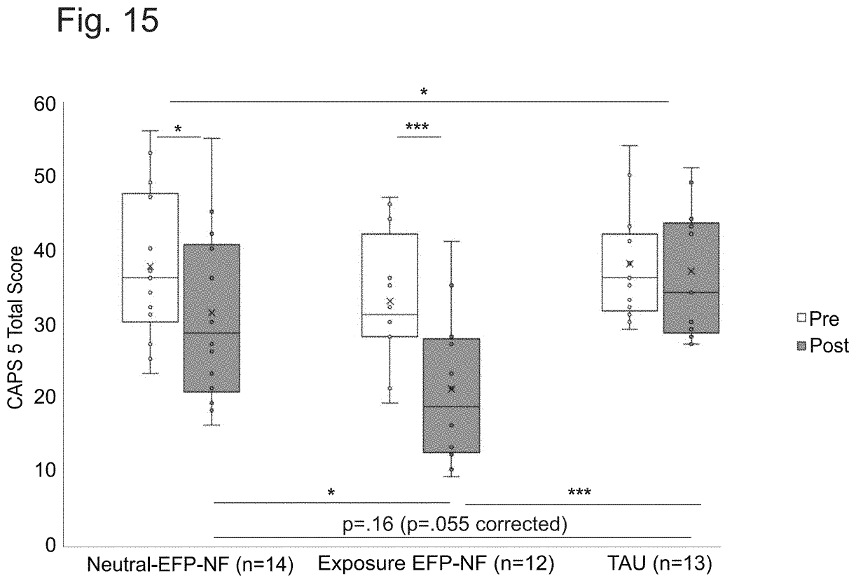

FIG. 15 demonstrates changes in CAPS 5 total score between the different groups of the experiment. CAPS (clinically administered PTSD scale) is an established and common structured clinical interview assessing for PTSD symptom severity. As shown in the figure, patients who received EFP training demonstrated a significant reduction in their CAPS scores, compared to patients who were not trained. This reduction is larger for patients in the exposure group. In some embodiments, changes in CAPS 5 or other clinically administered PTSD scales are used to evaluate a condition of a subject following the EFP-NF training.

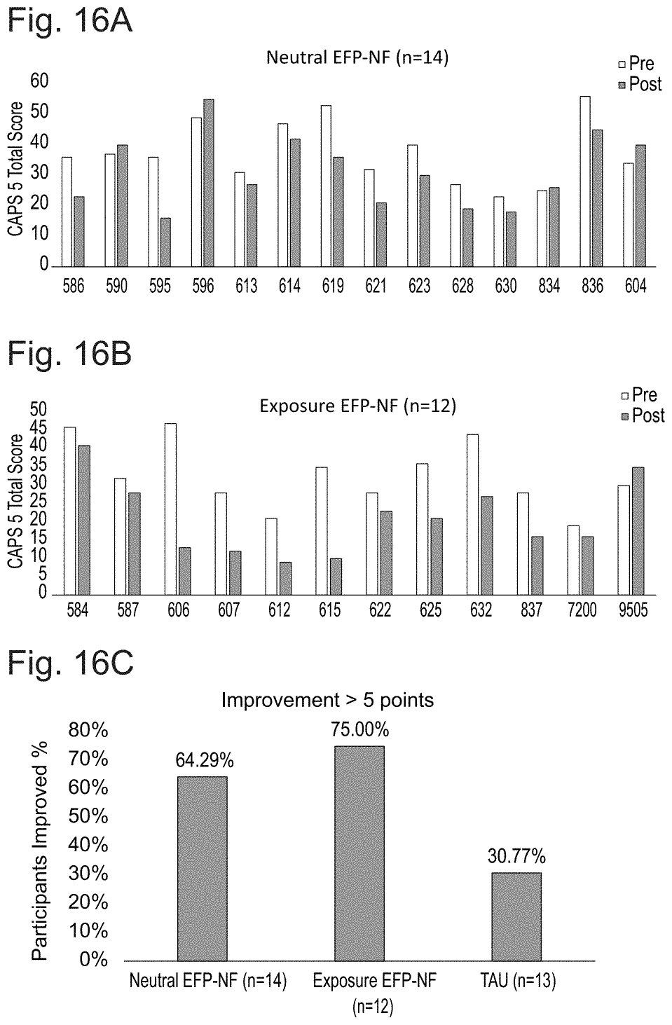

FIGS. 16A, 16B and 16C show individual CAPS results of patients in the neural context group (FIG. 16A) and the exposure group (FIG. 16B). FIG. 16C shows the percentage of participants who showed a reduction of more than 5 points in CAPS scores following intervention. This shows that the majority of patients in the exposure group (75%) had a meaningful reduction of CAPS scores following intervention.

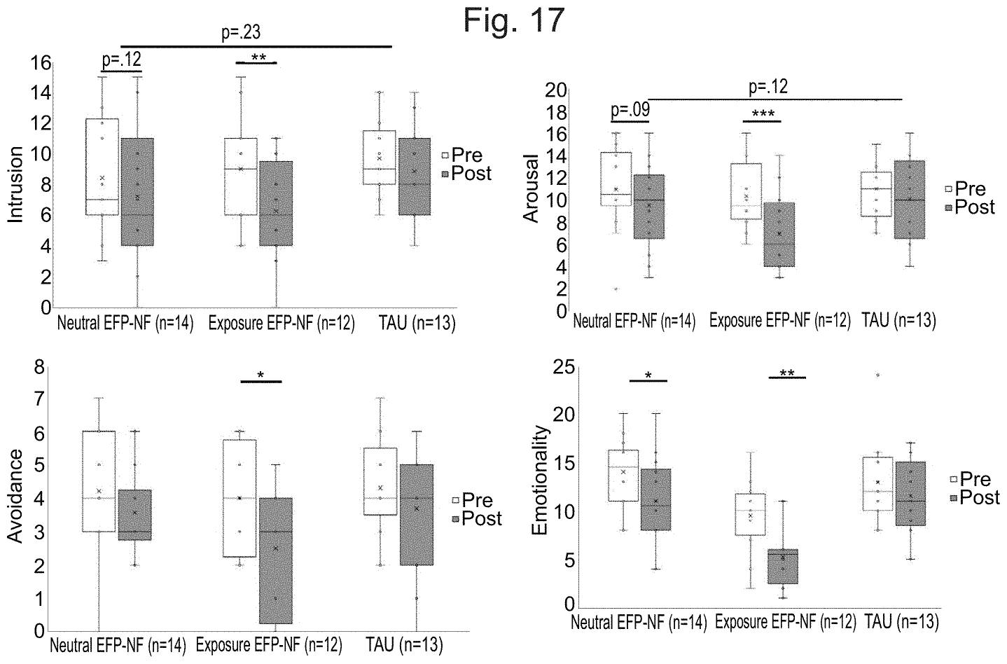

FIG. 17 describes changes in CAPS 5 subscales between the different groups. These subscales separately assess for PTSD symptom clusters: avoidance, Intrusion, Arousal and Cognitive and emotional alterations. In some embodiments, changes in CAPS 5 subscales are used to evaluate a condition or status of a subject following the EFP-NF.

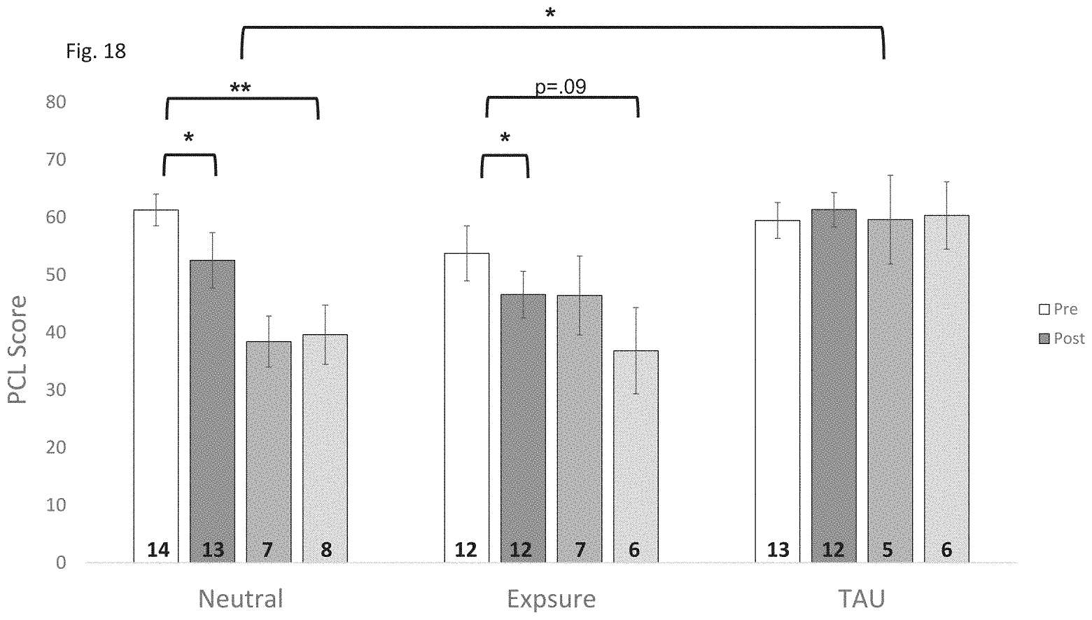

FIG. 18 describes changes in PCL score (self-report) between the different groups. PCL is an established and commonly used self-report measure that assesses PTSD symptoms. The results demonstrate that following intervention, patients in both EFP groups (Neutral group and Exposure group) showed a reduction of PTSD symptoms, compared to patients that did not receive the intervention (TAU group). This reduction in PTSD scores was preserved 3 and 6 months following the intervention. These results demonstrate that the effect of EFP intervention is lasting in time.

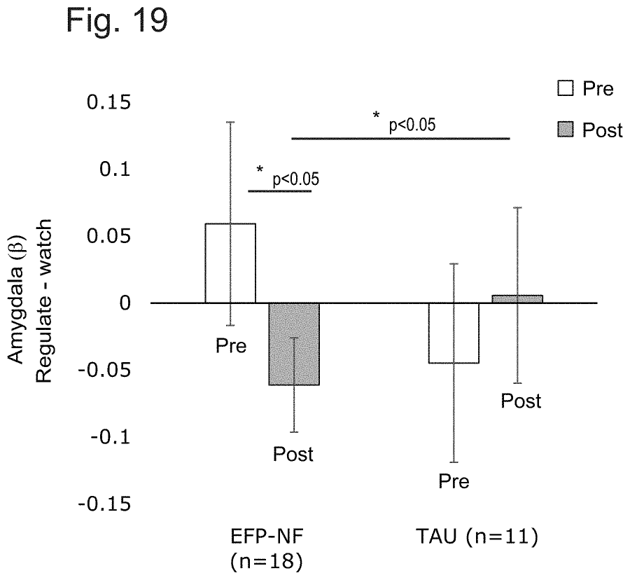

FIG. 19 describes changes in fMRI measures between the different groups before (left column—annotated as “pre”) and after (right column-annotated as “post”) the intervention. On the left is the first fMRI measure of the patients. On the right is their learning in real time fMRI on the post intervention session (i.e. the delta of the second and first cycles of that session. This figure demonstrates that patients who trained using the amygdala EFP were also successful at down regulating their BOLD amygdala activity following EEG training sessions, compared to participants who did not train using the EFP (Treatment as usual (TAU) group that did not receive NF training). In this figure you can see that patients who underwent Amygdala-EFP_NF treatment performed significantly better after treatment than before on Amygdala-MRI-NF. This means that patients in the treatment group were better than no-treatment group in down regulating their amygdala BOLD activity, after training with EFP-NF. In this figure Neutral (n=10)+Trauma (n=8) vs TAU. Pre: regulate—watch, only first cycle. Post: regulate—watch, mean of all 2 cycles.

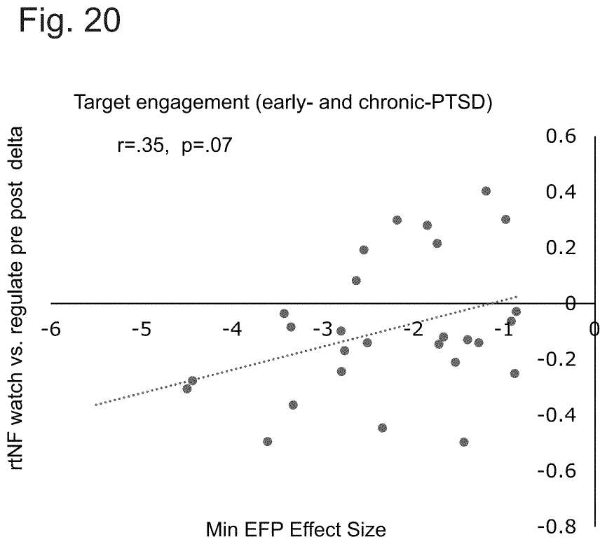

FIG. 20 demonstrates a correlation between EFP training and subsequent BOLD activation during real-time fMRI neurofeedback. This positive correlation demonstrates that participants who were very successful at down regulating their EFP signal during training sessions (i.e. overall best performance session) were also better at down regulating their amygdala BOLD signal during real-time neurofeedback following intervention. FIG. 20 shows that a change in EFP-NF is correlated with best change in EFP-NF (disregarding number of EFP-NF sessions).

Validation Experiment Summary

The validation experiment was directed to study modulation of processes underlying mental disorders via Neurofeedback Technologies fMRI Informed EEG as a Reliable neurofeedback probe for Limbic Modulation in PTSD.

Results summary: Relative to control-EFP-NF, Amyg-EFP-NF resulted in improved amygdala-BOLD down-regulation both in healthy participants (n=38) and PTSD patients (n=29). Lastly, to prove its feasibility as scalable NF probe, amygdala related behavioral modifications following repeated Amyg-EFP NF training, was tested. In healthy participants (n=˜150) the results show that relative to control-NF, Amyg-EFP-NF resulted in a larger reduction of alexithymia and improved emotion regulation as measured by an emotional Stroop task. In addition, results with PTSD patients (n=40) showed that Amyg-EFP-NF led to a reduction in PTSD ratings (˜10 points in Caps-5) and that this reduction correlated to successful Amyg-EFP down-regulation.

Conclusions: the results demonstrate the feasibility of the use of EEG to target limbic activity for NF in healthy and patients.

### Exemplary Additional Validation Experiment

### Exemplary Methods

### Participants

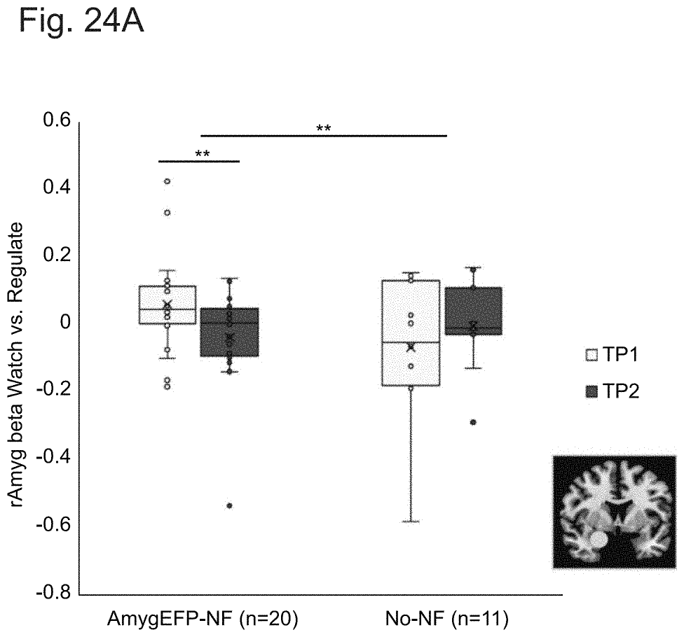

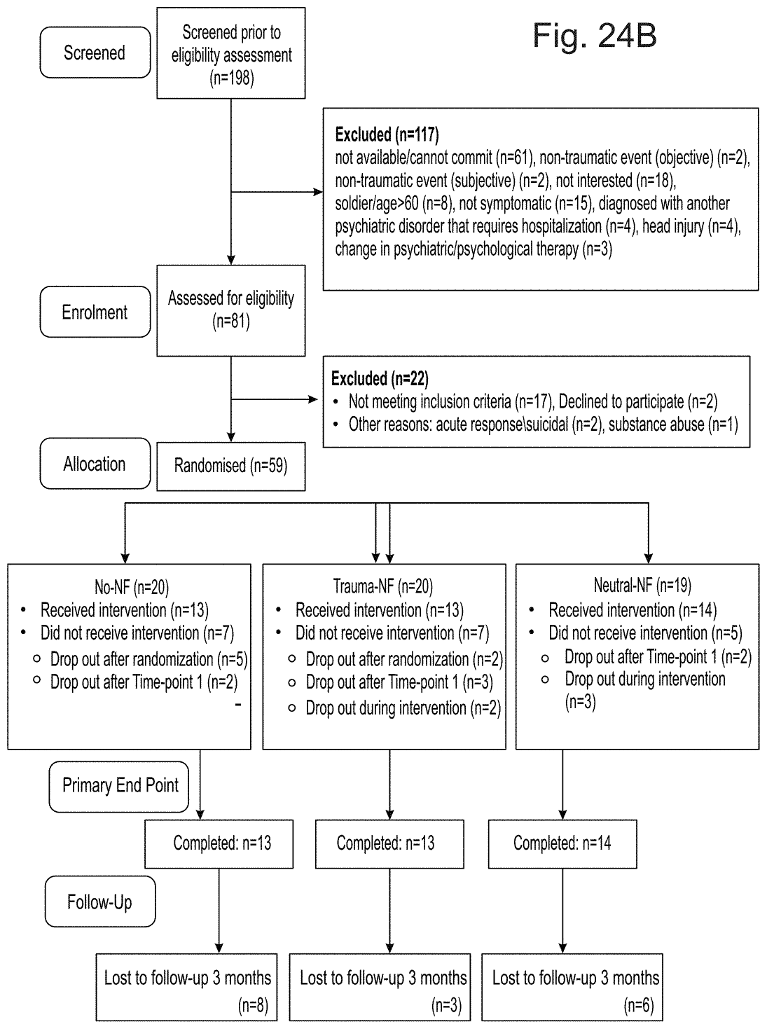

In the experiment, fifty-nine adults, aged 18-65 years, who met DSM-5 criteria for PTSD participated; forty completed the trial (Neutral-NF n=14, Trauma-NF n=13, No-NF n=13; see Supplementary FIG. 24B for CONSORT diagram). Recruitment venues included mental health clinics and social media advertisements. All patients underwent clinical assessment by a trained psychologist based on Structured Clinical Interview for DSM-IV axis I disorders (SCID) and Clinician Administered PTSD scale (CAPS-5) (see exclusion criteria in supplementary material). At baseline, groups did not significantly differ in mean age and time since trauma, nor in the proportion of females (see Table 1a,b). Patients gave written informed consent to participate and received financial compensation. The protocol was approved by the Tel-Aviv Sourasky Medical Center Institutional Review Board and registered at ClinicalTrials.gov.

TABLE 1a
Demographic Characteristics

| | CAPS-5 | Time Since | | Gender (% |
|---|---|---|---|---|
| | Score TP1 | Trauma (years) | Age | females) |
| Trauma-NF | 32.83 (2.67) | 8.71 (9.95) | 40.25 (21.96) | 58.33% |
| Neutral-NF | 37.84 (2.56) | 6.62 (6.27) | 37.66 (10.71) | 40% |
| No-NF | 37.92 (2.56) | 8.49 (8.89) | 32 (8.66) | 38.46% |

TABLE 1b
Trauma Type by Group

| | Trauma-NF | Neutral-NF | No-NF |
|---|---|---|---|
| | (n = 12) | (n = 15) | (n = 13) |
| Assault | | 4 | 4 |
| Car accident | 7 | 3 | 4 |
| Combat | 1 | 3 | 2 |
| Life threatening injury | 1 | 1 | 1 |
| Repeated exposure | | 1 | 1 |
| Terror | 2 | 2 | |
| Traumatic grief | | 1 | |
| Witness trauma | 1 | | 1 |

Table 1.a. Demographic Characteristics. Randomization was balanced by age (< or >40 years) and time since trauma (< or >5 years). To compare groups on baseline characteristics, one-way ANOVA and chi-square test were performed. CAPS-5 Score at TP1: F_{(2,35)}=1.22, p=0.306; Time Since Trauma: F_{(2,36)}=0.16, p=0.84; Age: F_{(2,36)}=2.01, p=0.14; Gender χ2(2)=1.08, p=0.58.

### General Procedure

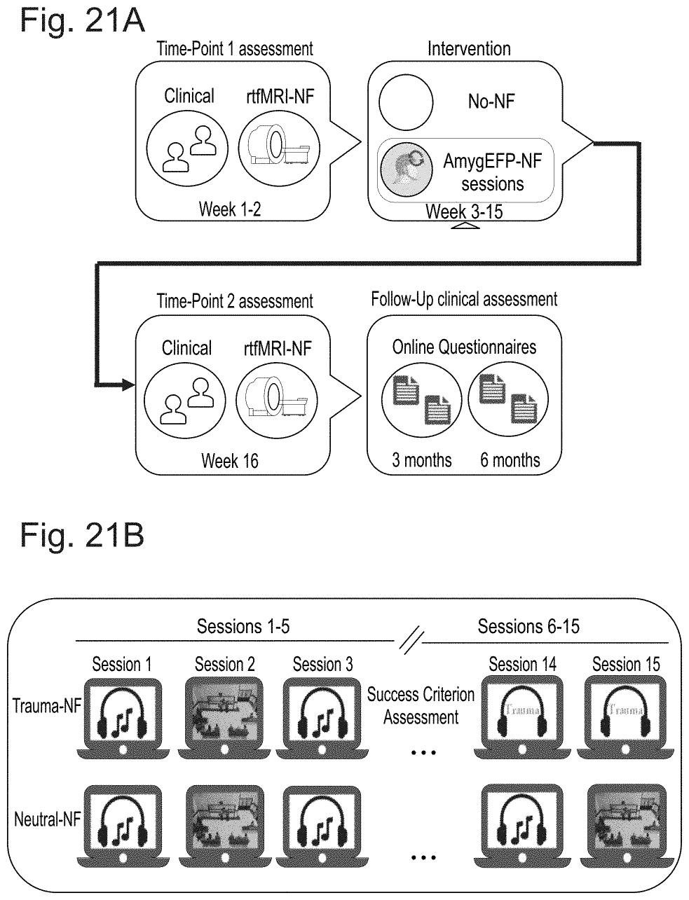

In the experiment and in some embodiments, participants were assessed before and immediately after the intervention (TP1, TP2) and also three- and six months following its completion (see FIG. 21A). In the experiment and in some embodiments, for example shown in FIGS. 1, 2A and 2G, primary clinical outcome measures included CAPS-5 interview and PTSD Checklist Questionnaire (PCL) (27). Secondary clinical outcome measures included State-Trait Anxiety Inventory (STAI) (28) and Beck Depression Inventory-II (BDI-II) (29) in order to further clinically characterize patients, as well as Toronto Alexithymia Scale (TAS-20) (30) and Emotion Regulation Questionnaire (ERQ) (31) in order to evaluate emotional regulation abilities. Following TP1, patients were randomly assigned to each of the three study groups: Trauma-NF, Neutral-NF, and No-NF. There were no differences between groups at baseline in any of the clinical measures (total CAPS-5, PCL, STAI, BDI-II, TAS-20 and ERQ). In some embodiments, at least one of the clinical outcome measures, the scales and the questionnaires described above are also used for assessment of a PTSD patient prior to training, for example as described at block 204 in FIG. 2A, and at blocks 250, 252 and 254 in FIG. 2G.

Reference is now made to FIG. 21A depicting the Study Procedure. Clinical assessments, structural and rtfMRI-NF scans were performed at Time-point 1 (TP1) and Time-point 2 (TP2). Follow-up assessments were performed at 3 and 6 months following TP2 using online questionnaires.

Reference is now made to FIG. 21B, depicting an intervention Protocol in the experiment and in some embodiments of the invention. The intervention phase included 15 training sessions, starting twice weekly for two weeks and then once a week (total of 13 weeks). Each session lasted approximately 40 minutes including preparation time. Patients were seated in a quiet room in front of a computer screen wearing headphones. The first 5 sessions were identical for both groups and employed two types of feedback interfaces in an interleaved manner: a neutral auditory interface and a multimodal scenario interface on the following session. The auditory interface was a jazz music piece with no lyrics while the multimodal animated scenario interface depicted a virtual hospital waiting room including characters sitting down or walking around and approaching a receptionist (see Supplementary material for detailed interface description). The auditory sessions included five consecutive training blocks each for three-minutes. The volume of sound could increase or decrease as a function of AmygEFP signal power (in units of 10 dB each). The multimodal scenario session type also included five consecutive blocks, each consisting of one-minute active baseline (unrest level of the room was set to 75% and not affected by participant modulation), three-minute NF during which patients trained to modulate their brain signal and received feedback by means of the room's scenario (for further details see Multimodal animated scenario online calculation in Supplementary material). Patients were guided to reduce the auditory tone or relax the animated scenario by using a variety of self-generated mental strategies. Patients in the Neutral-NF group continued to train using these interfaces in an interleaved manner until the completion of 15 intervention sessions, while patients in the Trauma-NF group who met the criteria (see Trauma-NF criteria in Supplementary material) moved on to the second phase of training which involved the trauma-related interface.

### AmygEFP-NF Training Protocol

In the experiment and in some embodiments, each NF session began with a three-minute rest recording of baseline AmygEFP signal, followed by five three-minute training blocks with either auditory or multimodal feedback interface (FIG. 21B). Each training block concluded with patient debriefing on the techniques they employed and a graphic display depicting their AmygEFP activity throughout the block (i.e. NF success). The procedure for Trauma-NF group was identical to that of the Neutral-NF group in the first sessions until they reached the criteria to begin trauma-narrative NF (around session 7-8, see Trauma-NF criteria in Supplementary). In the trauma-related NF sessions the feedback indicating AmygEFP signal was the volume of the trauma-narrative recording. A successful reduction of AmygEFP signal reduced the volume of the trauma-narrative. Each session began with a three-minute rest recording of baseline AmygEFP signal, followed by one block of neutral auditory NF and then three blocks of NF with the recorded trauma-narrative. Debriefing and a graphical display of individual performance also followed each block.

AmygEFP amplitude was calculated on-line based on data recorder from the Pz channel using an in-house algorithm (22, 23). AmygEFP signal down-regulation was assessed by calculating a personal NF success index for each subject in each session (i.e. average of 5 NF blocks minus average of baseline blocks, divided by average baseline standard deviation) using the following formula:

$$NF success = \frac{mean regulate - mean baseline}{SD baseline}$$

A desired result would be lower AmygEFP values during regulation, than during baseline, resulting in a more negative NF success index (See supplementary for EEG data recording details).
rtfMRI-NF Paradigm

In the experiment, MRI scans were performed in a 3.0T Siemens MRI system (MAGNETOM Prisma) using a 20-channel head coil. Preprocessing and statistical analysis were performed using BrainVoyager QX version 2.8 (Brain Innovation) (see Supplementary). To demonstrate target-engagement of AmygEFP-NF training, all patients completed rtfMRI-NF at TP1 and TP2 using a visual interface (see Supplementary FIG. 2). Feedback was derived from a 6-mm sphere in Talairach space in the right amygdala (coordinates, 20, −5, −14). The level of amygdala activity was represented by the speed of a skater (based on (24, 26). Patients were instructed to lower the skater's speed by using any mental strategy they see fit. Decreased speed corresponded with decreased amygdala BOLD activity during NF relative to baseline (see Supplementary Methods).

### Statistics

R version 1.70, SPSS version 20 and STATISTICA version 10 were used for statistical analysis. Multiple comparisons correction was performed using FDR (<0.05) for each hypothesis.

### Exemplary Results

### AmygEFP-NF Learning Effects

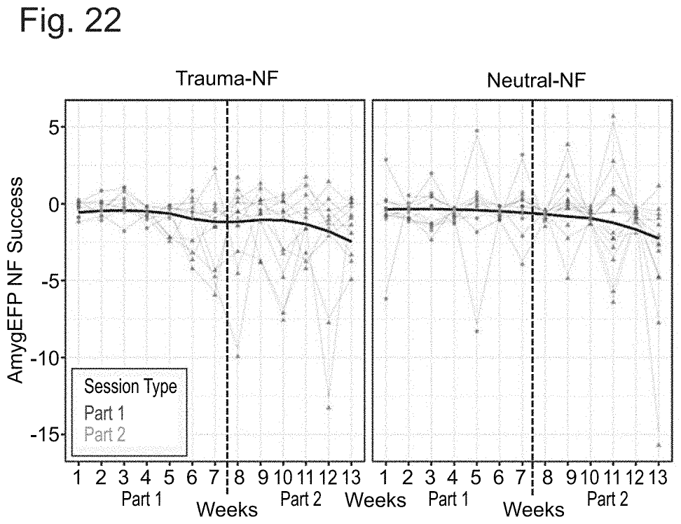

To demonstrate feasibility, patients ability to learn to volitionally down-regulate their AmygEFP signal was examined. A patient-repeated measurements nested mixed-model analysis were applied: Fixed effects of Group (Neutral-NF, Trauma-NF) and Time (13 weekly training sessions) were fitted. To enable exploratory analysis of the impact of context on differences in modulation between AmygEFP groups, the fixed effect for Session Type was also fitted (Part 1: Neutral-NF sessions 1-7, Trauma-NF sessions prior to exposure; Part 2: Neutral-NF sessions 8-13, Trauma-NF sessions including exposure). Considering the nested structure of the study, we also included patient-specific random effects, including the random effects of Time nested within Group. Evaluation of statistical significance was performed using likelihood ratio tests (LRT) and post-hoc analysis for pairwise comparisons. Results demonstrated that both treatment groups showed greater down regulation of AmygEFP signal as indicated by lower values during regulate compared to baseline blocks. Session Type (Part 1, 2) was tested via LRT and showed no random interaction with Time and Group factors (χ²(1)=1.77, p=0.18), yet showed a significant fixed-main effect for change in AmygEFP signal over sessions (χ²(1)=4.91, p=0.026). Post-hoc analysis comparing Session Type (Part 1 vs. Part 2) further clarified that the main effect of Time was driven by greater down-regulation of brain signal during the latter part of the intervention (p=0.057). As hypothesized, treatment groups demonstrated improved modulation throughout intervention sessions (see FIG. 22).

Reference is now made to FIG. 22 depicting a NF Learning Effect. Trauma-NF group (left panel) and Neutral-NF (right panel) individual AmygEFP-NF success as a function of intervention week. Session Type is indicated in orange (Trauma-NF group weekly sessions with a neutral interface; Neutral-NF group weekly sessions 1-7) and green (Trauma-NF group sessions with trauma-narrative; Neutral-NF group sessions 8-13). Black line denotes predictions of time-point means made by the reported nested mixed-model, and smoothed via Loess regression. Results show a significant fixed-main effect for Session Type, demonstrating change in AmygEFP signal over sessions (χ2(1)=4.91, p=0.026) and specifically greater down-regulation during the latter part of the intervention.

Further exploratory analysis was aimed at assessing the impact of trauma-narrative on modulation by comparing learning in Trauma-NF vs. Neutral-NF. LRT showed no significant difference between the two groups (χ²(3)=1.64, p=0.65). Post-hoc comparison between groups conditioned on Session Type, showed no difference between groups in either Part 1 (p=0.48), or Part 2 (p=0.48). Altogether this exploratory analysis revealed that modulation was similar between treatment groups, both in the initial and later stages of intervention. Nevertheless, in order to assess change in modulation for each Group specifically, Session Type was tested via LRT, conditioning on Group followed by post-hoc analysis. This analysis revealed a significant decrease in AmygEFP-NF success index in Part 2 Session Type, in both groups (Neutral-NF p=0.06, Trauma-NF p=0.007). Importantly, the monotonically decreasing curves from start to end, in each group, explicitly show that improvement throught the intervention, had a steeper slope in the Trauma-NF, compared to Neutral-NF group (see FIG. 22).

### AmygEFP-NF Clinical Efficacy

In the experiment, changes in primary clinical outcome measures (total CAPS-5 and PCL) from TP1 to TP2 between treatment and No-NF groups, were compared using 2-way repeated measures ANOVAs with Time (TP1, TP2) as within- and Group (Neutral-NF, Trauma-NF and No-NF) as between-subject independent variables.

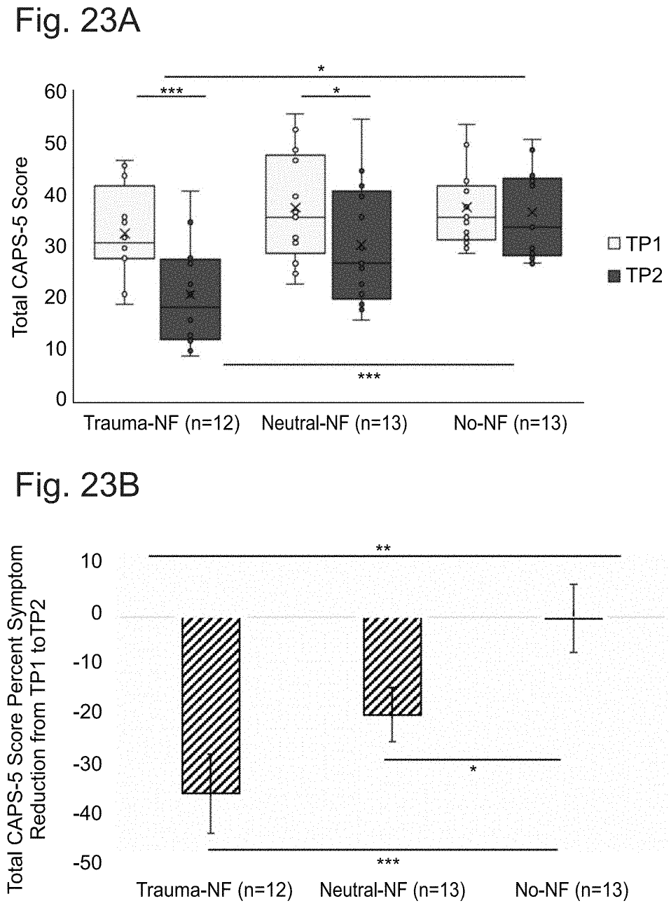

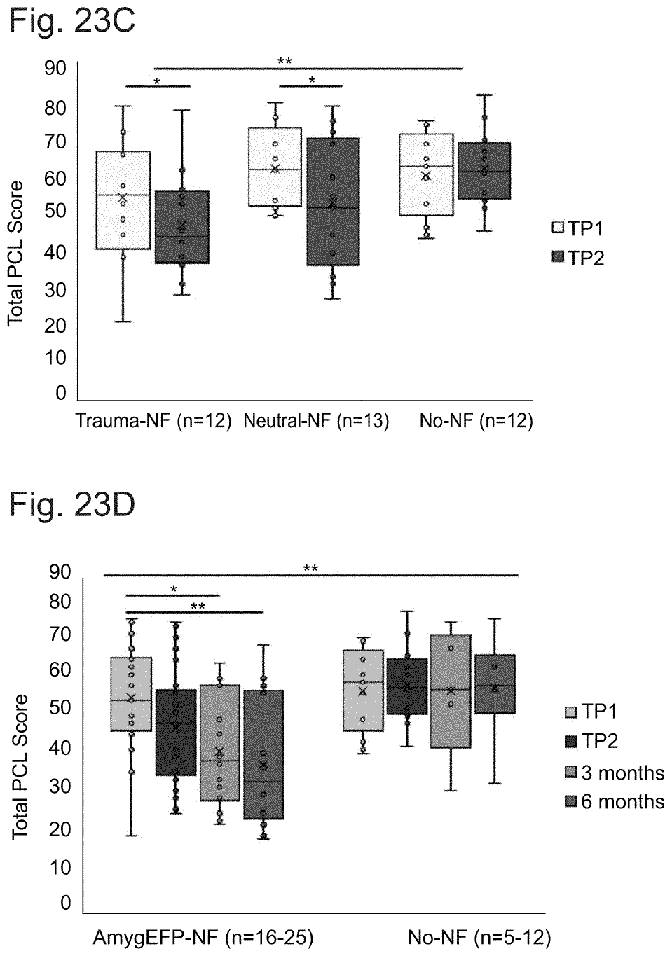

CAPS-5 total score analysis revealed greater improvement for AmygEFP-NF groups compared to No-NF group and more so for the Trauma-NF group (Time by Group interaction F_{(2,35)}=3.99, p=0.02, ηₚ²=0.18; Group main effect F_{(2,35)}=4.94, ηₚ²=0.02, ηₚ²=0.22; Time main effect F_{(1,35)}=18.20, ηₚ²=0.0007, ηₚ²=0.34; see FIG. 23A). Planned contrasts revealed clinical improvement for treatment but not for the No-NF group (simple effect of Time per Group: Neutral-NF group F=7.22, p=0.02; Trauma-NF group F=18.12, p=0.0007; No—NF group F=0.13, p=0.71). Further analysis showed that at TP2 there was greater improvement in Trauma-NF compared to No-NF group (F=14.83, p=0.0001), while the difference between Neutral-NF and No-NF was not significant (F=2.39, p=0.15). Efficacy of treatment was further evaluated via Number Needed to Treat (NNT) index based on achieving loss of PTSD diagnosis according to CAPS-5 at TP2; comparing treatment groups to No-NF NNT=3.9 (Neutral-NF vs. No-NF, NNT=6.5; Trauma-NF vs. No-NF, NNT=2.7). We further showed that the percent of symptom reduction in total CAPS-5 score was different between groups (Group main effect, F_{(2,35)}=6.67, p=0.01, ηₚ²=0.27; FIG. 23B). In comparison to No-NF group which showed no improvement (−0.23%), Trauma-NF showed the largest decrease (−35.13%; F=13.25, p=0.0008), followed by Neutral-NF group (−19.48%; F=4.203, p=0.04).

Similarly, PCL analysis revealed, as hypothesized, that patients in treatment groups improved more than the No-NF group (Time by Group interaction F_{(2,34)}=4.403, p=0.02, ηₚ²=0.20; Time main effect F_{(1,34)}=6.14, p=0.02, ηₚ²=0.15; see FIG. 23C). Planned contrasts revealed a PCL reduction from TP1 to TP2 in the Neutral-NF group (Δ=−9.07; F=9.13, p=0.01) and in the Trauma-NF group (Δ=−7.16; F=5.25, p=0.02), but not in the No-NF group (Δ=−2.84; F=0.92, p=0.36). NNT for the change in PCL score, comparing treatment groups to No-NF showed NNT=2.4.

To examine the long-term effect of treatment we analyzed PCL scores at three and six months after TP2. Response rate in follow-up assessment was moderate (61.2%). To ensure unbiased analysis, analysis was conducted on data derived only from patients with four time points of PCL. Two-way repeated-measures ANOVA was conducted for PCL total score with Time (TP1, TP2, 3 and 6-months follow-up assessments) as within- and Group (AmygEFP-NF and No-NF) as between-subject independent variables. Results showed a decrease in PTSD symptoms over time, more so for the treatment arm than in the No-NF group (Time by Group interaction F_{(3,48)}=5.83, p=0.001, ηp2=0.26). Post-hoc analyses revealed a PCL reduction from TP1 to 3 months follow up (F=7.9, p=0.012) and from TP1 to 6 months follow up (F=8.34, p=0.008) in the treatment arm (FIG. 23D). Taken together these results confirm our second and third hypotheses and demonstrate clinical efficacy of the AmygEFP intervention in PTSD patients, compared to No-NF group.

Reference is now made to FIGS. 23A-23D depicting clinical outcome measures. FIG. 23A: Total CAPS-5 (Clinician Administered PTSD Scale) score reflecting the severity of PTSD symptoms at TP1 and TP2 assessments for Trauma-NF, Neutral-NF and No—NF groups. Box represents first and third quartiles; the line represents the median while “x” represents the mean; whiskers depict minimum and maximum outside the first and third quartiles. Results demonstrate a significant reduction of CAPS-5 score following the intervention in the Neutral- and Trauma-NF groups, but not in the No-NF control group (Time by Group interaction F_{(2,35)}=3.99, p=0.02, ηp2=0.18; Time main effect F_{(1,35)}=18.20, p=0.0007, ηp2=0.34; Simple effect of Time per Group: Neutral-NF group F=7.22, p=0.02; Trauma-NF group F=18.12, p=0.0007; No—NF group F=0.13, p=0.71). At TP2 there was greater improvement in Trauma-NF compared to No-NF group (F=14.83, p=0.0001), while the difference between Neutral-NF and No-NF was not significant (F=2.39, p=0.15).

FIG. 23B: Total CAPS-5 Score Percent Symptom Reduction from TP1 to TP2. Results demonstrate a significant difference between groups in percent of total symptom reduction according to CAPS-5 (Group main effect, F_{(2,35)}=6.67, p=0.01, ηp2=0.27). In comparison to No-NF group which showed no improvement (−0.23%), Trauma-NF showed the largest decrease (−35.13%; F=13.25, p=0.0008), followed by Neutral-NF group (−19.48%; F=4.203, p=0.04).

FIG. 23C: Total PCL (PTSD Checklist) assessed at TP1 and TP2. Results show a significant decrease in subjective PTSD severity following the intervention in both treatment groups, compared to No-NF control (Time by Group interaction F_{(2,34)}=4.403, p=0.02, ηp2=0.20; Time main effect F_{(1,34)}=6.14, p=0.02, ηp2=0.15). Planned contrasts revealed a PCL reduction from TP1 to TP2 in the Neutral-NF group (Δ=−9.07; F=9.13, p=0.01) and in the Trauma-NF group (Δ=−7.16; F=5.25, p=0.02), but not in the No-NF group (Δ=−2.84; F=0.92, p=0.36).

FIG. 23D: Total PCL throughout the study: TP1, TP2, three and six months following the intervention. Results show a decrease in subjective PTSD severity following the intervention in both treatment groups, compared to No-NF control, throughout the study (Time by Group interaction F_{(3,48)}=5.83, p=0.001, ηp2=0.26). Post-hoc analyses revealed a PCL reduction from TP1 to 3 months follow-up (F=7.9, p=0.012) and from TP1 to 6 months follow up (F=8.34, p=0.008) in the treatment arm.

rtfMRI-NF

To test target engagement of AmygEFP-NF in probing the amygdala, rtfMRI-NF success in down-regulating amygdala BOLD signal, was assessed. Amygdala activity was compared between patients in AmygEFP treatment arm and No-NF arm via a random-effects general linear model for amygdala signal change (regulate vs. watch, see FIG. 24A and Supplementary 1.4 for details of model). A 2-way repeated measures ANOVA was conducted with right amygdala beta values as a dependent variable, and Group (AmygEFP-NF, No-NF) and Time (TP1, TP2) as independent variables. Six patients were originally not included in the fMRI testing due to MR incompatibility, three additional patients were excluded due to missing data at one TP and two additional patients were excluded due to excessive head motion, resulting in n=20 in AmygEFP-NF group and n=11 in No-NF group, for the rtfMRI analysis. Results demonstrated as expected, greater reduction in amygdala-B OLD signal after intervention in the treatment arm than in the control arm (Time by Group interaction F_{(1,29)}=10.31, p=0.004, ηₚ²=0.26). Planned comparisons for change over time per group showed the desired NF effect for the treatment arm (F=10.85, p=0.004), but not for the No-NF control arm (F=2.41, p=0.13) (see FIG. 4). This finding supports target engagement for the AmygEFP-NF treatment in the right amygdala.

Reference is now made to FIG. 24A depicting rtfMRI-NF Target Engagement. Average beta values obtained from the right amygdala used as the target for regulation during rtfMRI-NF. The model included six regressors for each condition in each cycle (Baseline, NF and Washout) in TP1 and TP2 separately. Regressors were convolved with a canonical hemodynamic response function. Additional nuisance regressors included the head-movement realignment parameters. Regulation is depicted by the difference between regulate versus attend trials, shown for AmygEFP-NF and No-NF arms at TP1 (the first cycle) and TP2 (averaged two cycles). Time by Group interaction F_{(1,29)}=10.31, p=0.004, ηp2=0.26; Time simple main effect for AmygEFP-NF, F=10.85, p=0.004 and for No-NF, F=2.41, p=0.13.

### Discussion

The current study presents a randomized controlled trial of a process-based NF intervention, aimed at amygdala down-regulation in a scalable manner in PTSD patients. In accordance with our first hypothesis, patients in the treatment arm demonstrated neuromodulation learning throughout NF sessions, with Trauma-NF showing a steeper improvement compared to Neutral-NF. In line with our second and third hypotheses, patients in treatment arm showed clinical improvements, compared to No-NF arm, as indicated by decreased CAPS-5 and PCL measures following the intervention; Trauma-NF group showed the largest decrease. Intriguingly, follow-up at 3 and 6 months of PCL point to further decrease in symptoms only in the treatment arm. Finally, we demonstrated target engagement of the AmygEFP-NF intervention as indicated by superior down-regulation of amygdala BOLD signal during a single session of rtfMRI-NF for patients in the treatment arm compared to No-NF arm. This is the first demonstration of clinical effects in PTSD using fMRI-inspired EEG-NF training, while targeting the trauma-related process in a personalized manner.

### Learning Effects

Patients learned to down-regulate AmygEFP signal through repeated training sessions, with feedback of disorder-specific or non-specific content. Learning mainly occurred at the latter part of the training (FIG. 2). Interestingly, the personalized trauma-narrative feedback did not interfere with learning and might have even accelerated it. It is possible that the gradual removal of a more aversive cue (i.e. reducing the volume of trauma-narrative) led to increased reward and more rapid learning (32). Future works could test this option by introducing enhanced exposure with success, or separating exposure and reward using intermittent feedback. Our protocol used 15 NF sessions, designed to match the protocols of other common psychotherapies, however it is possible that a different number of sessions could achieve as good or better learning and clinical effects. Considering the vast heterogeneity of NF learning abilities, it might be advantageous to customize protocol for individual patients or try to predict clinical benefits from initial NF success (33-35).

### Clinical Effect

Results showed a large clinical effect for chronic PTSD; total CAPS-5 score change showed a large effect size in the Trauma-NF group (Cohen's D 1.229; Hedges's g 0.828), a medium effect size in the Neutral-NF group (Cohen's D 0.636; Hedges's g 0.4306) and a large effect size when considering the treatment arm together (Cohen's D 0.853; Hedges's g 0.591). Importantly, clinical efficacy was driven by a large symptom reduction following treatment (Trauma-NF-35.13%, Neutral-NF-19.48%), compared to No-NF (−0.23%; FIG. 3b). The AmygEFP intervention showed similar clinical benefits to commonly used cognitive-behavioral therapies. For example, a meta-analysis showing that prolonged exposure therapy (PE) in PTSD outperforms control conditions with a large effect size (Hedges's g=1.08, 95% CI 0.69 to 1.46) (21), and Watts et al. (2013) which showed an overall effect size of g=0.81 (95% CI 0.71 to 0.91) in 112 randomized clinical trials, including psychotherapy (g=1.14 (95% CI 0.97-1.3)) and somatic treatments (g=1.24 (95% CI 0.35-2.13)). Our results further showed that for loss of PTSD diagnosis according to CAPS-5 at TP2, both treatment groups in comparison to No-NF, yielded NNT=3.9 (and 2.4 for total PCL score). These results are compatible with reported NNT≤4 for achieving loss of PTSD diagnosis following psychotherapy (37). Intriguingly, in the follow-up assessments at three and six months after the intervention, patients showed continued improvement in PTSD severity as indicated by PCL (FIG. 3d). This is in line with accumulating reports on continued improvement weeks after the end of NF interventions (25, 38). One explanation is that NF could be equated to skill learning that is practiced in patients' daily lives (intentionally or automatically). Another possibility is that consolidation and reconsolidation processes, that are typical in NF learning, occur after training completion through synchronization of the targeted brain circuits. In sum, the clinical results of our study demonstrate the feasibility and effectivity of processing traumatic content while directly targeting one of its underlying limbic related mechanisms.
One could argue that the large effect found for Traum-NF is due to combined treatments of NF and repeated exposure to traumatic content, in line with the known effect of exposure-based psychotherapies (21). We argue, however, that this cannot explain the extent of our results since the Trauma-NF did not follow common practice of prolonged exposure therapy; it was a relatively low-dose (7 sessions), short duration (3-minute segments vs. 40-60 min), and limited content (only a gist of the traumatic memory and not an extended narrative), and was also not self-generated. To note, dropout rates during the AmyEFP-NF were 10% and 15.7% for Trauma-NF and Neutral-NF, respectively (see Supplementary FIG. 1 for CONSORT diagram), while exposure-based treatments often result in higher dropout rates of up to 40% (39). The AmygEFP intervention requires neither verbal and interpersonal interactions nor repeated long exposures, yet shows similar clinical benefits and lower dropout rates.

### NF Mechanism Considerations

Notably, not only PTSD symptoms improved over time in the treatmen arm and were maintained during follow-up, but also anxiety and depression symptoms (see Supplementary 2.2). PTSD has a high rate of comorbidity mainly with major depression, anxiety disorders and substance use disorders (40, 41). These findings highlight the importance of assessing comorbidity in PTSD, but also support our idea that the AmygEFP-NF probes an underlying mechanism of the disorder; emotion regulation, which is relevant transdiagnostically. Future studies could delineate clinical effects with regard to other processes underlying PTSD and comorbid pathologies; e.g. reward processing (through upregulation of mesolimbic circuit) or cognitive control (through upregulation of attention circuit), all associated with depression, substance abuse and/or PTSD.
Results demonstrated target engagement of the AmygEFP-NF intervention, by showing improved down-regulation of amygdala BOLD signal during rtfMRI-NF for patients in the treatment arm relative to No-NF arm. It would be advantageous to demonstrate an association between AmygEFP-NF intervention and rtfMRI-NF target engagement. However, the complexity of acquiring data due to this clinical population and high study costs, led to this study to be underpowered to find such associations. Another way to explore mechanism specificity is to demonstrate a correlation between the AmygEFP intervention and clinical outcomes. Similar to Goldway et al. (2018) this study did not demonstrate a correlation between AmygEFP learning and clinical outcomes (see Supplementary 2.3). This is in opposition to our finding in a-priori healthy soldiers that underwent similar training for increasing stress resilience (26). One explanation may be that clinical changes in patients may not be linearly related to level of NF proficiency. Rather, skills are acquired and incorporated in behavioral repertoire (42). Thus, skill acquisition and not the level of proficiency, is the driving factor of clinical change. Yet, evidence favoring mechanism specificty could be found in our finding that patients who trained using the trauma-narrative interface showed larger improvement relative to those trained with Neutral-NF in several symptom clusters of CAPS-5 (intrusion, avoidance and arousal, see Supplementary 2.1). Future studies, with a larger sample size, could explore specific process-related outcome measures (e.g. cognitive testing, emotional challenges etc.) and pursue learning-specific manifestations as an additional indication for success, especially in patients.

### Conclusion

We showed that PTSD patients successfully down-regulated their AmygEFP signal while exposed to individually tailored traumatic content. In a randomized controlled manner we demonstrated a medium size effect for clinical improvement following AmygEFP-NF treatment in PTSD patients, compared to No-NF controls. Intriguingly, the effect of clinical improvement was larger for patients trained with trauma-narrative as the feedback interface, supporting process-based NF approach. Importantly, target-engagement and generalizability were further demonstrated by showing that patients in treatments groups exhibited greater down-regulation of amygdala BOLD signal during rtfMRI, with a different interface, compared to No-NF. To conclude, the current study demonstrated the feasibility and clinical efficacy of a scalable neurofeedback intervention—based on neural mechanism underlying PTSD. It widens the horizon for more brain-guided treatments in psychaitry that are neuroscientifically inspired.

### REFERENCES FOR THE EXEMPLARY ADDITIONAL STUDY

1. Karam E G, Friedman M J, Hill E D, et al.: Cumulative Traumas and Risk Thresholds: 12-Month Ptsd in the World Mental Health (wmh) Surveys. Depress Anxiety 2014; 31:130-142
2. Fenster R J, Lebois LAM, Ressler K J, et al.: Brain circuit dysfunction in post-traumatic stress disorder: from mouse to man. Nat Rev Neurosci 2018; 19:535
3. Hayes J P, Hayes S M, Mikedis A M: Quantitative meta-analysis of neural activity in posttraumatic stress disorder. Biol Mood Anxiety Disord 2012; 2:9
4. Lanius R A, Vermetten E, Loewenstein R J, et al.: Emotion Modulation in PTSD: Clinical and Neurobiological Evidence for a Dissociative Subtype. Am J Psychiatry 2010; 167:640-647
5. Shin L M, Rauch S L, Pitman R K: Amygdala, Medial Prefrontal Cortex, and Hippocampal Function in PTSD. Ann N Y Acad Sci 2006; 1071:67-79
6. Admon R, Lubin G, Stern O, et al.: Human vulnerability to stress depends on amygdala's predisposition and hippocampal plasticity. Proc Natl Acad Sci 2009; 106:14120-14125
7. Admon R, Lubin G, Rosenblatt J D, et al.: Imbalanced Neural Responsivity to Risk and Reward Indicates Stress Vulnerability in Humans. Cereb Cortex 2013; 23:28-35
8. Admon R, Milad M R, Hendler T: A causal model of post-traumatic stress disorder: disentangling predisposed from acquired neural abnormalities. Trends Cogn Sci 2013; 17:337-347
9. Etkin A, Buechel C, Gross J J: The neural bases of emotion regulation. Nat Rev Neurosci 2015; 16:693-+
10. Sitaram R, Ros T, Stoeckel L, et al.: Closed-loop brain training: the science of neurofeedback. Nat Rev Neurosci 2017; 18:86
11. Young K D, Siegle G J, Zotev V, et al.: Randomized Clinical Trial of Real-Time fMRI Amygdala Neurofeedback for Major Depressive Disorder: Effects on Symptoms and Autobiographical Memory Recall. Am J Psychiatry 2017; 174:748-755
12. Paret C, Kluetsch R, Ruf M, et al.: Down-regulation of amygdala activation with real-time fMRI neurofeedback in a healthy female sample. Front Behav Neurosci 2014; 8:UNSP 299
13. Paret C, Ruf M, Gerchen M F, et al.: fMRI neurofeedback of amygdala response to aversive stimuli enhances prefrontal-limbic brain connectivity. NeuroImage 2016; 125:182-188
14. Zotev V, Krueger F, Phillips R, et al.: Self-Regulation of Amygdala Activation Using Real-Time fMRI Neurofeedback. PLoS ONE 2011; 6:e24522
15. Herwig U, Lutz J, Scherpiet S, et al.: Training emotion regulation through real-time fMRI neurofeedback of amygdala activity. Neuroimage 2019; 184:687-696
16. Nicholson A A, Rabellino D, Densmore M, et al.: The neurobiology of emotion regulation in posttraumatic stress disorder: Amygdala downregulation via real-time fMRI neurofeedback: Amygdala Down-Regulation in PTSD. Hum Brain Mapp 2017; 38:541-560
17. Zotev V, Phillips R, Misaki M, et al.: Real-time fMRI neurofeedback training of the amygdala activity with simultaneous EEG in veterans with combat-related PTSD. NeuroImage Clin 2018; 19:106-121
18. Gerin M I, Fichtenholtz H, Roy A, et al.: Real-Time fMRI Neurofeedback with War Veterans with Chronic PTSD: A Feasibility Study. Front Psychiatry 2016; 7:111
19. Nicholson A A, Rabellino D, Densmore M, et al.: Intrinsic connectivity network dynamics in PTSD during amygdala downregulation using real 0 time fMRI neurofeedback: A preliminary analysis. Hum Brain Mapp 2018; 39:4258-4275
20. Lubianiker N, Goldway N, Fruchtman-Steinbok T, et al.: Process-based framework for precise neuromodulation. Nat Hum Behav 2019; 1
21. Powers M B, Halperin J, Ferenschak M, et al.: A meta-analytic review of prolonged exposure for posttraumatic stress disorder. Clin Psychol Rev 2010; 30:635-641
22. Meir-Hasson Y, Kinreich S, Podlipsky I, et al.: An EEG Finger-Print of fMRI deep regional activation. Neuroimage 2014; 102:128-141
23. Meir-Hasson Y, Keynan J N, Kinreich S, et al.: One-Class FMRI-Inspired EEG Model for Self-Regulation Training. Plos One 2016; 11:e0154968
24. Keynan J N, Meir-Hasson Y, Gilam G, et al.: Limbic Activity Modulation Guided by Functional Magnetic Resonance Imaging?Inspired Electroencephalography Improves Implicit Emotion Regulation. Biol Psychiatry 2016; 80:490-496
25. Goldway N, Ablin J, Lubin 0, et al.: Volitional limbic neuromodulation has a multifaceted clinical benefit in Fibromyalgia patients. Neuroimage 2018; 186:758-770
26. Keynan J N, Cohen A, Jackont G, et al.: Electrical fingerprint of the amygdala guides neurofeedback training for stress resilience. Nat Hum Behav 2019; 3:63
27. Weathers F W, Litz B T, Herman D S, et al.: The PTSD Checklist (PCL): Reliability, validity, and diagnostic utility., inannual convention of the international society for traumatic stress studies. San Antonio, Tex., 1993
28. Speilberger C D, Gorsuch R L, Lushene R, et al.: Manual for the State Trait Anxiety Inventory (Form Y). Consult Psychol Press Palo Alto 1983;
29. Beck A T, Steer R A, Carbin M G: Psychometric properties of the Beck Depression Inventory: Twenty-five years of evaluation. Clin Psychol Rev 1988; 8:77-100
30. Bagby R M, Parker J D., Taylor G J: The twenty-item Toronto Alexithymia scale-I. Item selection and cross-validation of the factor structure—ScienceDirect. J Psychosom Res 1994; 38:23-32
31. Garnefski N, Kraaij V: The Cognitive Emotion Regulation Questionnaire. Eur J Psychol Assess 2007; 23:141-149
32. Ferrucci L, Nougaret S, Brunamonti E, et al.: Effects of reward size and context on learning in macaque monkeys. Behav Brain Res 2019; 372:UNSP 111983
33. Alkoby O, Abu-Rmileh A, Shriki O, et al.: Can We Predict Who Will Respond to Neurofeedback? A Review of the Inefficacy Problem and Existing Predictors for Successful EEG Neurofeedback Learning. Neuroscience 2018; 378:155-164
34. Paret C, Goldway N, Zich C, et al.: Current progress in real-time functional magnetic resonance-based neurofeedback: Methodological challenges and achievements. NeuroImage 2019; 202:116107
35. Kadosh K C, Staunton G: A systematic review of the psychological factors that influence neurofeedback learning outcomes. Neuroimage 2019; 185:545-555
36. Watts B V, Schnurr P P, Mayo L, et al.: Meta-analysis of the efficacy of treatments for posttraumatic stress disorder. J Clin Psychiatry 2013; 74:e541-50
37. Jonas D E, Cusack K, Forneris C A, et al.: Psychological and pharmacological treatments for adults with posttraumatic stress disorder (PTSD)2013;
38. Rance M, Walsh C, Sukhodolsky D G, et al.: Time course of clinical change following neurofeedback. Neuroimage 2018; 181:807-813
39. Imel Z E, Laska K, Jakupcak M, et al.: Meta-Analysis of Dropout in Treatments for Posttraumatic Stress Disorder. J Consult Clin Psychol 2013; 81:394-404
40. Kessler R C, Sonnega A, Bromet E, et al.: Posttraumatic Stress Disorder in the National Comorbidity Survey. Arch Gen Psychiatry 1995; 52:1048-1060
41. Campbell D G, Felker B L, Liu C-F, et al.: Prevalence of Depression-PTSD Comorbidity: Implications for Clinical Practice Guidelines and Primary Care-based Interventions. J Gen Intern Med 2007; 22:711-718
42. Ramos-Murguialday A, Broetz D, Rea M, et al.: Brain-Machine Interface in Chronic Stroke Rehabilitation: A Controlled Study. Ann Neurol 2013; 74:100-108

### Supplementary Material for the Exemplary Additional Study

### 1. Method

### 1.1. Participants

Reference is now made to FIG. 24B depicting a CONSORT flow chart. Drop-out rates by group were: 26.3% in the Neutral-NF, 25% in the Trauma-NF and 10% in the no-NF group. One additional participant who was randomized to the Trauma-NF group did not meet the criteria described in ‘Trauma-related NF interface and procedure’ and was thus excluded from Trauma-NF group analyses.

In the experiment and in some embodiments of the invention the exclusion criteria included current pregnancy, major medical or neurological disorders, psychosis, major depression disorder, schizophrenia and serious suicidal ideations. Patients who were currently in psychotherapy and/or were receiving pharmacological treatments were included in the study, on the condition that no change was made to their treatment plan in the last three months and in the time remaining to the completion of the study.

### 1.2. General Procedure

TABLE 1
Clinical Outcome Measures

| | | TP1 | | | TP2 | | | 3 Month Follow-up | | | 6 Month Follow-up | | |
|---|---|---|---|---|---|---|---|---|---|---|---|---|---|
| | | | | Std. | | | Std. | | | Std. | | | Std. |
| | | N | Mean | Err. | N | Mean | Err. | N | Mean | Err. | N | Mean | Err. |
| CAPS-5 | Trauma- | 12 | 32.83 | 2.67 | 12 | 20.91 | 2.99 | | | | | | |
| | NF | | | | | | | | | | | | |
| | Neutral- | 13 | 37.84 | 2.56 | 13 | 30.61 | 2.87 | | | | | | |
| | NF | | | | | | | | | | | | |
| | No-NF | 13 | 37.92 | 2.56 | 13 | 36.92 | 2.87 | | | | | | |
| PCL | Trauma- | 12 | 53.75 | 16.49 | 12 | 46.58 | 14.01 | 10 | 46.1 | 16.56 | 8 | 38.87 | 18.65 |
| | NF | | | | | | | | | | | | |
| | Neutral- | 13 | 61.38 | 11.1 | 13 | 52.3 | 17.05 | 8 | 40.125 | 11.84 | 8 | 41.12 | 17.31 |
| | NF | | | | | | | | | | | | |
| | No-NF | 13 | 59.46 | 11.23 | 12 | 61.33 | 10.33 | 5 | 59.6 | 17.24 | 6 | 60.33 | 14.3 |
| STAI | Trauma- | 12 | 53 | 15.22 | 12 | 45.16 | 15.77 | 9 | 46.55 | 16.78 | 8 | 46.37 | 17.35 |
| | NF | | | | | | | | | | | | |
| | Neutral- | 13 | 60 | 10.8 | 12 | 50 | 12.22 | 8 | 45.87 | 12.73 | 8 | 44.87 | 12.66 |
| | NF | | | | | | | | | | | | |
| | No-NF | 12 | 52.66 | 15.45 | 11 | 55.18 | 13.89 | 5 | 58.8 | 13.49 | 6 | 65.5 | 12.25 |
| BDI-II | Trauma | 12 | 41.5 | 14.56 | 12 | 36.33 | 12.99 | 10 | 33 | 12.11 | 9 | 42.44 | 12.84 |
| | NF | | | | | | | | | | | | |
| | Neutral | 13 | 47.3 | 9.96 | 12 | 38.33 | 12.93 | 8 | 32 | 7.87 | 8 | 33 | 10.81 |
| | NF | | | | | | | | | | | | |
| | No-NF | 12 | 45.83 | 11.49 | 11 | 45.54 | 15.65 | 5 | 46.2 | 17.72 | 6 | 50 | 12.39 |
| ERQ | Trauma- | 12 | 27.66 | 9.36 | 12 | 32.08 | 5.83 | 10 | 31.2 | 6.01 | 9 | 29.33 | 5.78 |
| Reappraisal | NF | | | | | | | | | | | | |
| | Neutral- | 13 | 25.69 | 7.79 | 12 | 33.25 | 6.9 | 8 | 32.62 | 6.82 | 8 | 31.12 | 8.67 |
| | NF | | | | | | | | | | | | |
| | No-NF | 12 | 31.08 | 9.65 | 11 | 28.27 | 9.26 | 5 | 33.8 | 6.9 | 6 | 27.83 | 14.1 |
| ERQ | Trauma- | 12 | 14.66 | 6.24 | 12 | 13.25 | 6.001 | 10 | 12.4 | 5.56 | 9 | 12 | 5.74 |
| Suppression | NF | | | | | | | | | | | | |
| | Neutral- | 13 | 17.15 | 4.05 | 12 | 15.58 | 4.88 | 8 | 12.75 | 5.97 | 8 | 14.25 | 5.7 |
| | NF | | | | | | | | | | | | |
| | No-NF | 12 | 18.75 | 5.77 | 11 | 18.36 | 5.4 | 5 | 19.8 | 3.03 | 6 | 19 | 7.15 |
| TAS-20 | Trauma- | 12 | 52.83 | 16.12 | 12 | 49.91 | 13.37 | 9 | 50 | 14.9 | 9 | 47 | 13.25 |
| | NF | | | | | | | | | | | | |
| | Neutral- | 12 | 59.16 | 11.75 | 12 | 56.16 | 16.85 | 7 | 51.42 | 15.82 | 7 | 51 | 18.86 |
| | NF | | | | | | | | | | | | |
| | No-NF | 10 | 56.8 | 10.35 | 9 | 58.55 | 10.85 | 5 | 57.8 | 14.06 | 6 | 59.33 | 15.27 |

Table 1. Clinical Outcome Measures. Table 1 depicts results of each of the clinical measures that were used to evaluate clinical change, by group, at four time points (TP1, TP2, 3 Months and 6 Months follow up). To examine differences in TP1 between groups one-way ANOVA was conducted with Group (Trauma-NF, Neutral -NF, No-NF) as independent variables. Analysis did not reveal differences between groups at baseline in any of the clinical measures: CAPS-5 (F_{(2,35)} = 1.22, p = .306, ηₚ² = .06), PCL (F_{(2,35)} = 1.13, p = .33, ηₚ² = .06), STAI (F_{(2,34)} = 1.12, p = .33, ηₚ² = .06), BDI-II (F_{(2,34)} = .76, p = .47, ηₚ² = .04), TAS-20 (F_{(2,31}) = .71, p = .49, ηₚ² = .04), ERQ reappraisal (F_{(2,34)} = 1.15, p = .32, ηₚ² = .06), ERQ suppression (F_{(2,34)} = 1.74, p = .19, ηₚ² = .09).

Exemplary multimodal animated scenario online calculation, in the experiment and in some embodiments—The scenario features sounds of chatter and commotion of a busy emergency room. The scenario can gradually change from a resting state (all the people are seated and the sound volume is low) to an agitated state (people coming up to the receptionist and loudly protesting) and back again. The overall unrest level of the room is determined by the EFP signal power. The ratio between characters sitting down and protesting at the counter is considered to be a two-state Boltzmann distribution, whose evolution is driven by a ‘virtual temperature’ whose value is derived from the momentary value of the targeted signal power (AmygEFP). The scenario uses the probability (P value) of a momentary signal value during regulate to be sampled under the previous attend distribution. This P value is used to determine the probability of virtual characters to be moving in the virtual room, with the character distribution updated accordingly. A matching soundtrack recorded inside a real hospital complements the system output. Three alternative soundtracks with different agitation levels were produced and switched according to the signal value. During the attend condition, 75% of the characters congregate at the front desk while expressing their frustration through body and verbal language. The system is implemented using the Unreal Development Kit game engine, which controls relevant animations (walking, sitting, standing and protesting), as well as their transitions for individual characters.

Exemplary Trauma-NF criteria in the experiment and in some embodiments of the invention—Patients who succeed in lowering AmygEFP signal during training in three out of the last five sessions, or in four out of six total sessions, continued. Patients who were not successful continued to train in the neutral context for the remaining sessions. In the second phase of training, patients in the trauma-NF group trained while receiving feedback with the context of their traumatic story. In the first session, patients were interviewed about the traumatic event that met DSM-5 criterion A using a commonly used methodology (Shalev, Orr & Pitman, 1993; Rauch, 1996) in order to produce a scripted detailed chain of events, including thoughts, feelings, sensations and contextual information. The interview was edited and then recorded as a three-minute audio segment (second-person male voice in present tense). Following the interview patients trained again with a neutral NF interface. Patients who were successful at down regulation of their AmygEFP signal during this session continued on to train with the trauma-narrative feedback in the following sessions.
Shalev A Y, Orr S P, Pitman R K: Psychophysiologic assessment of traumatic imagery in Israeli civilian patients with posttraumatic stress disorder. Am J Psychiatry 1993; 150:620-624
Rauch S L: A Symptom Provocation Study of Posttraumatic Stress Disorder Using Positron Emission Tomography and Script-Driven Imagery. Arch Gen Psychiatry 1996; 53:380

### 1.3 Exemplary EEG Data Recording

EEG data was acquired using the V-Amp™ EEG amplifier (Brain Products™, Munich Germany) and the BrainCap™ electrode cap with sintered Ag/AgCI ring electrodes (Falk-Minow Services™, Herrsching-Breitburnn, Germany). Electrodes were positioned according to the standard 10/20 system. The reference electrode was between Fz and Cz. Raw EEG signal was sampled at 250 Hz and recorded using Brain Vision Recorder™ software (Brain Products). The baseline on auditory sessions was the initial rest period and in the multimodal animation scenario the baseline was the active baseline blocks in each training cycle.
1.4 Exemplary fMRI Data Acquisition and Processing
To allow high-resolution structural images, a T1-weighted three-dimensional (3D) sagittal MPRAGE pulse sequence (repetition time/echo time=1,860/2.74 ms, flip angle=8°, pixel size=1×1 mm, field of view=256×256 mm) was used. Functional whole-brain scans were performed in an interleaved top-to-bottom order, using a T2*-weighted gradient echo planar imaging pulse sequence (repetition time/echo time=2,500/30 ms, flip angle=82°, pixel size=2.3 mm, field of view=220×220 mm, slice thickness=3 mm, 42 slices per volume). Slice scan time correction was performed using cubic-spline interpolation. Head motions were corrected by rigid body transformations, using three translations and three rotation parameters, and the middle image served as a reference volume. Trilinear interpolation was applied to detect head motions, and sinc interpolation was used to correct them. The temporal smoothing process included linear trend removal and usage of a high-pass filter of 1/128 Hz. Functional maps were manually co-registered to corresponding structural maps and, together, they were incorporated into 3D data sets through trilinear interpolation. The complete data set was transformed into Talairach space and spatially smoothed with an isotropic 6-mm full width at half-maximum Gaussian kernel.
Exemplary rtfMRI-NF Paradigm

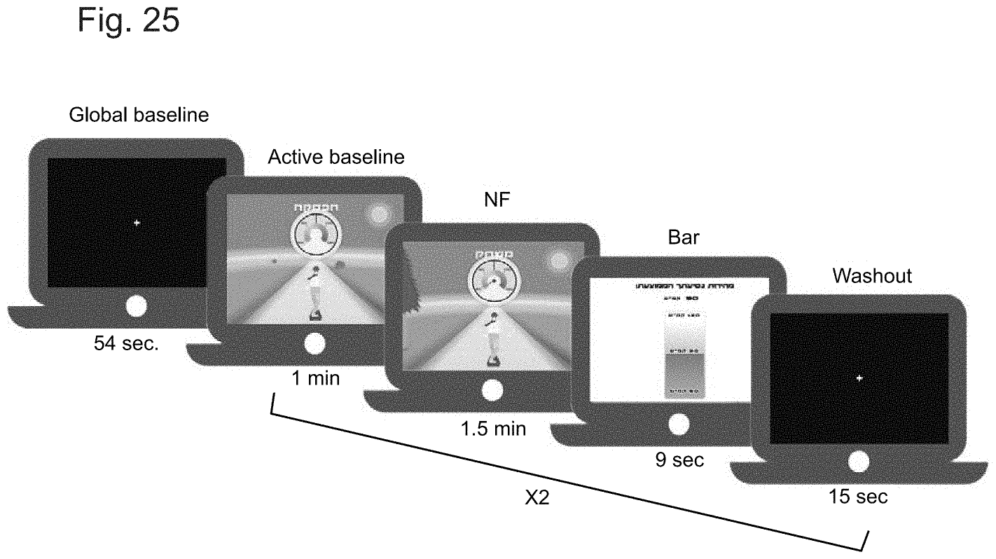

Reference is now made to FIG. 25, depicting an rtfMRI-NF paradigm in the study and in some embodiments. The visual feedback interface consisted of a 2D unimodal flash-based graphic, with an animated figure skating on a road. The NF paradigm included 5 conditions. During ‘Global baseline’ (54 sec.), which appeared only in the first block of each run, patients were instructed to fix their gaze on the cross. In the ‘Active baseline’ condition (60 sec.) the skateboard rider was riding at an average fixed speed and the speedometer was not presented. Patients were instructed to passively view the skateboard rider and to try not to engage in any mental activity other than watching. Patients were guided not to consider any mental strategies or previous successes or failures during this condition; stressing the importance of creating a significant mental difference between conditions. During the ‘Neurofeedback’ condition (90 sec.), patients were presented with a continuous feedback on their right amygdala BOLD activity embodied in the rider's changing speed accompanied by the speed display on the speedometer (online feedback calculation is detailed below). Instructions were to decrease the speed of the skateboard rider by practicing any mental strategy they see fit. In the ‘Bar’ condition (9 sec.), subjects were informed of their average speed, representing their success in the last NF block relative to its previous baseline block, which assisted them to decide whether to keep practicing a successful strategy, or to replace a failed one. In the ‘Washout’ condition (15 sec.), subjects were instructed to cease all contemplating and self-reflecting, and prepare for the next baseline block. The paradigm consisted of two cycles with all four conditions (Active baseline, Neurofeedback, Bar, Washout) in addition to the ‘Global baseline’ that appeared once in the beginning.

Exemplary rtfMRI-NF Paradigm Online Calculation

Momentary beta weights of the predefined amygdala ROI (averaged across all voxels of the ROI) were extracted online using Turbo Brain voyager 3.0 (Brain Innovation). The beta weights were then transferred to MATLAB™. For each TR, right amygdala activity was first converted to standardized score:

$$zBold ( t ) = \frac{B ( t ) - μ ( {B}_{BL} )}{σ ( {B}_{BL} )}$$

Where B(t) is the right amygdala BOLD activity value at time point t, μ(B_BL) is the mean BOLD value during the previous ‘Active baseline’. Values varied from −4 std. below baseline average to 4 std. above average. Next, each value was transferred into a speed scale using the following formula:

Speed(t)=60+15*zBold(t)

This equation results in the fact that the right amygdala BOLD value that equals the previous ‘Active baseline’ average value, is set to 60 km/h and each increase or decrease of 1 std. results in a 15 km/h change. The −4 to 4 limit translates to a possible speed range of 0-120 km/h. Moreover, in order to make the NF paradigm more interactive, flexible for modulation and challenging, an updating principle was incorporated. During NF block, represented values were limited by an interval of 4 std., resulting in a 60 km/h interval in speed scale. In the first block of the first run, the presented interval was symmetric around the average speed (60 km/h): −2 to 2 (30 km/h to 90 km/h). In each consecutive run, the interval was updated based on the patients' success in the previous run, according to the following steps:
1. During the first cycle the interval in the NF screen is set to −2 to 2.
2. When the NF screen terminates a success index of the current block is calculated thus (n indicating the cycle's ordinal number):

$${Success}_{Index ( n )} = \frac{μ ( {B}_{NF ( n )} )}{pooled ( σ ( {B}_{BL ( n )} ) + σ ( {B}_{NF ( n )} ) )}$$

By dividing the mean NF standardized value with the pooled average, we introduce the index not only with the subject's ability to successfully regulate his or her right amygdala activity during the NF block, but also his or her ability to maintain it as constant as possible during the ‘Active baseline’ block.
3. The interval [lower limit upper limit] for the next cycle (n+1) is updated to be [Lower lim=(success_index(n)−learning_rate) Upper lim=(success_index(n)−learning_rate+4)] The learning rate parameter is preset to: learning rate=1.
   This process is aimed at enabling significant regulation in one run to result in further regulation in the following run. This makes the NF paradigm more challenging and dynamic and pushes towards maximizing regulation.

### 2. Results

### 2.1 AmygEFP-NF Clinical Efficacy

Reference is now made to FIGS. 2H-2K, depicting changes in sub-scales in CAPS 5, the experiment and in some embodiments.

The clinical effect with respect to type of feedback (trauma vs. neutral) was explored by examining changes in CAPS-5 subscale in the treatment groups (i.e. Intrusion, Avoidance, Alterations in Cognition and Mood, Arousal). Two-way repeated measures ANOVAs were performed for each CAPS-5 subscale with Time (TP1, TP2) as within- and Group (Neutral-NF and Trauma-NF) as between-subject independent variables. All scales demonstrated a similar improvement over time with no Time by Group interaction (Time main effect: Avoidance F_{(1,23)}=15.75, p=0.0006, ηp2=0.406; Intrusion F_{(1,23)}=9.73, p=0.004, ηp2=0.29; Alteration in Cognition and Mood F_{(1,23)}=15.43, p=0.0004, ηp2=0.41; Arousal F_{(1,23)}=16.54, p=0.004, ηp2=0.41, see FIGS. 2H-2K). Although no significant interaction were found, in further post-hoc tests it was evident that for all subscales there was a smaller decrease in symptoms for the Neutral-NF relative to the Trauma-NF group (Time simple main effect: Intrusion: Trauma-NF Δ=−1.5, F=6.34, p=0.01; Neutral-NF Δ=−1.07, F=3.54, p=0.072; Avoidance: Trauma-NF Δ=−2.75, F=12.46, p=0.001; Neutral-NF Δ=−1.54, F=4.22, p=0.051; Alteration in Cognition and Mood: Trauma-NF Δ=−4.25, F=10.204, p=0.004; Neutral-NF Δ=−3.23, F=6.38, p=0.01; Arousal: Trauma-NF Δ=−3.42, F=16.11, p=0.0005; Neutral-NF Δ=−1.39, F=2.86, p=0.103).

FIGS. 2H-2K depict CAPS-5 subscales, in the experiment and in some embodiments. Intrusion (FIG. 2H), Avoidance (FIG. 2J), Alterations in Cognition and Mood (FIG. 2I), and Arousal (FIG. 2K). Scores reflect change in severity of PTSD symptom subscales between TP1 and TP2 for Trauma-NF and Neutral-NF groups. Box represents first and third quartiles; the line represents the median while “x” represents the mean; whiskers depict minimum and maximum outside the first and third quartiles.

### 2.2 Exemplary Secondary Clinical Outcomes

We employed a set of secondary clinical assessments based on self-rating questionnaires: alexithymia; TAS-20 (n=33), depression; BDI-II (n=35), anxiety; STAI (n=35) and emotion regulation; ERQ (n=35). Two-way repeated measures ANOVAs were conducted with Time (TP1, TP2) as within- and Group (Neutral-NF, Trauma-NF and No-NF) as between-subject independent variables.

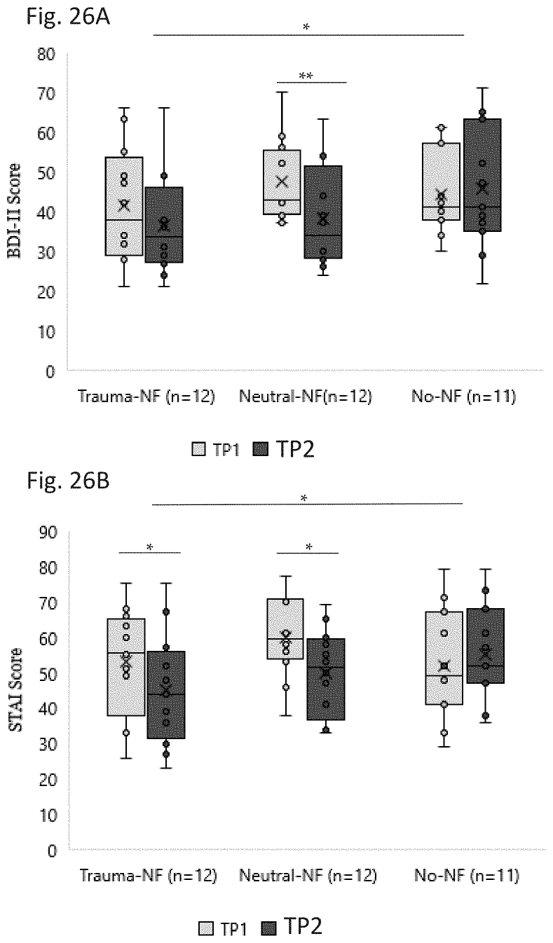

Results showed improvement in BDI-II scores over time for the treatment arm, compared to No-NF group (Time by Group interaction F_{(2,32)}=3.48, p=0.04, ηp2=0.17; Time main effect F_{(1,32)}=7.28, p=0.01, ηp2=0.18, FIG. 26A). Post-hoc analysis revealed reduced depression symptoms in the Neutral-NF group (Time simple main effect; F=11.14, p=0.002; 4=−11.92), and marginal reduction for Trauma-NF group (F=3.47, p=0.07; 4=−5.16), but no change in the No-NF group (F=0.19, p=0.66; 4=−4.08). Analysis of change in the STAI scale, showed reduced anxiety symptoms over time in the treatment arm but not in the control group (marginal Time by Group interaction F_{(2,32)}=3.28, p=0.0504, ηp2=0.17, Time main effect F_{(1,32)}=4.69, p=0.03, ηp2=0.12, FIG. 26B.). Post-hoc analysis revealed reduced anxiety symptoms in the Neutral-NF group (Time simple main effect; F=6.8, p=0.01; 4=−9.91), and in the Trauma-NF group (F=4.24, p=0.04; 4=−7.83), but no change in the No-NF group (F=0.67, p=0.41; 4=3.27). Analysis of change in alexithymia and emotion regulation did not reveal differences between treatment and No-NF arms: TAS-20 showed no Time by Group interaction (F_{(2,30)}=0.66, p=0.52, ηₚ²=0.04); ERQ score was divided into two scales: reappraisal and suppression, a significant Time by Group interaction was found for reappraisal (F_{(2,32)}=5.03, p=0.01, ηₚ²=0.23) but not for suppression (F_{(2,32)}=0.11, p=0.89, ηₚ²=0.007).

FIGS. 26A and 26B describe secondary clinical outcomes. Scores reflect change in depression (FIG. 26A) and anxiety (FIG. 26B) symptom between TP1 and TP2 for Trauma-NF, Neutral-NF and No—NF groups. Box represents first and third quartiles; the line represents the median while “x” represents the mean; whiskers depict minimum and maximum outside the first and third quartiles

### 2.3 Exemplary AmygEFP Learning and Clinical Improvement Associations

We examined whether clinical improvements observed in the treatment arm correspond with their NF learning effect. Pearson correlation coefficients were computed to assess the relationship between clinical change (decrease in total CAPS-5 and PCL scores) to AmygEFP learning (measured by average and minimal NF success index). One patient was removed due to values 2.5std below minimum AmygEFP NF success index. Results showed that only average and minimal AmygEFP NF success index were correlated (see Table 2).

TABLE 2
AmygEFP learning and clinical improvement associations

| Measure | 1 | 2 | 3 |
|---|---|---|---|
| (1) CAPS-5 total score | — | | |
| improvement | | | |
| (2) PCL total score | .12 | — | |
| improvement | (p = .59, | | |
| | n = 23) | | |
| (3) Average AmygEFP NF | −.12 | .06 | — |
| success index | (p = .57, | (p = .77, | |
| | n = 24) | n = 24) | |
| (4) Minimum AmygEFP NF | −.01 | −.27 | .86** |
| success index | (p = .96, | (p = .2, | (p = .000, |
| | n = 24) | n = 24) | n = 25) |

**Correlation is significant at p < .01 (two-tailed)

It is expected that during the life of a patent maturing from this application many relevant EEG electrodes will be developed; the scope of the term EEG electrodes is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for training a subject diagnosed with a stress disorder caused by a trauma, comprising:
selecting a challenge expected to trigger a symptom of said stress disorder in said subject;
exposing said subject to said challenge;
recording electrical signals generated by the brain of said subject by at least one electrode, in conjunction with said exposing;
processing said recorded electrical signals to estimate an activation level of at least one specific brain region;
presenting at least one indication of said estimated activation level to said subject, wherein said presenting comprises providing feedback to said subject by modifying said challenge according to said activation level of said at least one specific brain region;
repeating said recording, said processing and said presenting.

2. A method according to claim 1, wherein said exposing to said challenge affects an activation level of said at least one specific brain region.

3. A method according to claim 1, comprising:
identifying a relation between at least a portion of said recorded electrical signals and an electrical fingerprint indicating an activation level of said at least one brain region, and wherein said generating comprises generating said at least one indication based on said identified relation.

4. A method according to claim 1, wherein said at least one specific brain region is a brain region of a limbic system.

5. A method according to claim 1, wherein said challenge comprises a challenge selected to activate an amygdala and/or brain regions connected to the amygdala by a neural network.

6. A method according to claim 1, wherein said stress disorder comprises post-traumatic stress disorder (PTSD).

7. A method according to claim 1, wherein said challenge is selected to upregulate activation of said at least one specific brain region, and wherein said at least one indication is continuously presented according to an ability of said subject to downregulate said activation level.

8. A method according to claim 1, comprising:
instructing said subject to perform at least one exercise selected to affect an activation level of said at least one specific brain region.

9. A method according to claim 8, wherein said presenting comprises presenting said at least one indication according to an ability of said subject to modulate an activation level of said at least one specific brain region by performing said at least one exercise.

10. A method according to claim 1, comprising providing at least one EEG electrical fingerprint indicating an activity level of said at least one specific brain region, and wherein said processing comprises processing said recorded electrical signals with said at least one EEG electrical fingerprint to identify a relation between at least a portion of said electrical signals and said at least one EEG electrical fingerprint, and wherein said activation level of said at least one specific brain region is estimated based on said identified relation.

11. A method according to claim 1, wherein said providing feedback comprises continuously modifying an intensity or severity of said challenge according to said activation level of said at least one specific brain region.

12. A method according to claim 11, wherein said continuously modifying comprises lowering an intensity or severity of said challenge if an activation level of said at least one specific brain region is lowered, based on said processing.

13. A device for delivery of a stress disorder training, comprising:
a memory;
a control circuitry functionally coupled to said memory, configured to:
deliver to said subject a challenge configured to trigger at least one symptom of said stress disorder in said subject;
receive at least one electrical signal generated by the brain of said subject;
process said received electrical signals;
estimate using said processed received electrical signals, an activation level of at least one specific brain region;
provide at least one human detectable indication to said subject by modifying said challenge delivered to the subject based on the estimated activation level of said at least one specific brain region.

14. A device according to claim 13, comprising a user interface, wherein said user interface is used to select said challenge from a list of challenges stored in said memory.

15. A device according to claim 13, wherein said control circuitry is configured to process said effect to determine how to modulate said challenge.

16. A device according to claim 13, wherein said at least one electrical signal comprises an EEG signal.

17. A device according to claim 13, wherein said memory stores at least one EEG electrical fingerprint indicating an activity level of said at least one specific brain region, and wherein said control circuitry is configured to identify a relation between at least a portion of the received at least one electrical signal and said stored at least one EEG electrical fingerprint stored in said memory, and estimates said activation level of said at least one specific brain region based on said identified relation.

18. A device according to claim 17, wherein said at least one EEG electrical fingerprint comprises an EEG electrical fingerprint of an Amygdala, indicating an activity level of said Amygdala.

19. A device according to claim 13, wherein said modifying said challenge by said control circuitry comprises continuously modifying an intensity or severity of said challenge based on said estimated activation level of said at least one specific brain region.

20. A device according to claim 19, wherein said control circuitry is configured to continuously modify said challenge by lowering an intensity or severity of said challenge if an activation level of said at least one specific brain region is downregulated based on said estimating.
